(19) United States
(12) Patent Application Publication
(10) Pub. No.: US 2025/0019457 A1
(43) Pub. Date: Jan. 16, 2025

(54) TROP2 ANTIBODIES

(71) Applicant: iBio, Inc., Bryan, TX (US)

(72) Inventors: Dillon Phan, La Jolla, CA (US); Cory Schwartz, San Diego, CA (US); Matthew P. Greving, Rancho Santa Fe, CA (US); Cody A. Moore, Del Mar, CA (US); Tam Thi Thanh Phuong, San Diego, CA (US); Matthew Dent, San Diego, CA (US); Alexander T. Taguchi, San Diego, CA (US); Jiang Chen, San Diego, CA (US); Tom Sih-Yuan Hsu, Chino, CA (US); Domyoung Kim, San Diego, CA (US); Martin Brenner, San Diego, CA (US)

(21) Appl. No.: 18/758,187

(22) Filed: Jun. 28, 2024

Related U.S. Application Data
(60) Provisional application No. 63/506,268, filed on Jun. 5, 2023.
(60) Provisional application No. 63/515,375, filed on Jul. 25, 2023.

(51) Int. Cl. C07K 16/30; C07K 16/28

(52) U.S. Cl. CPC C07K 16/30; C07K 16/2809; C07K 2317/24; C07K 2317/622; C07K 2317/734

(57) ABSTRACT

Provided herein are anti-Trop 2 antibodies or binding fragments thereof that bind Trop 2, e.g., human Trop 2. The anti-Trop 2 antibodies of the disclosure are useful for the treatment of proliferative disorders or cells that express Trop 2 or mutant Trop 2. Also provided herein are methods of use for the anti-Trop 2 antibodies or binding fragments thereof, as well as bi-valent or multi-valent anti-Trop 2 antibodies or binding fragments thereof that may form complexes that attract immune effectors or binding to other cells, such as, a second antibody, antigen-binding of the second antibody or fragment thereof; a target-binding protein, a cytokine; a lectin; or a toxin.

### CROSS-REFERENCE TO RELATED APPLICATIONS

This application claims priority to U.S. Provisional Application Ser. No. 63/506,268, filed Jun. 5, 2023 and U.S. Provisional Application Ser. No. 63/515,375, filed Jul. 25, 2023, and, the entire contents of which are incorporated herein by reference.

### TECHNICAL FIELD OF THE INVENTION

This document relates to materials and methods for treating a cancer, and particularly to the use of anti-TROP2 and bispecific antibodies to reduce or eliminate cancer, and to treat proliferative diseases.

### INCORPORATION-BY-REFERENCE OF MATERIALS FILED ON COMPACT DISC

The application contains a Sequence Listing which has been submitted electronically in .XML format and is hereby incorporated by reference in its entirety. Said .XML copy, created on Jul. 18, 2024, is named “IBIO1041.xml” and is 272,948 bytes in size. The sequence listing contained in this .XML file is part of the specification and is hereby incorporated by reference herein in its entirety.

### BACKGROUND OF THE INVENTION

Without limiting the scope of the invention, its background is described in connection with TROP2 antibodies.

One such antibody is taught in U.S. Patent Application Nos. 20180271992 and 20180185351, filed by Cardillo and Goldenberg, entitled, “Treatment of high TROP-2 expressing triple negative breast cancer (TNBC) with sacituzumab govitecan (IMMU-132) overcomes homologous recombination repair (HRR) rescue mediated by Rad51” and “therapy of small-cell lung cancer (SCLC) with a topoisomerase-i inhibiting antibody-drug conjugate (ADC) targeting Trop-2”, respectively. These Applicants are said to teach the treatment of Trop-2 positive cancers with the combination of an anti-Trop-2 antibody-drug conjugate (ADC) and a Rad51 inhibitor, such as SN-38, wherein the ADC is sacituzumab govitecan. The ADC may be administered at a dosage of between 4 mg/kg and 16 mg/kg, preferably 4, 6, 8, 9, 10, 12, or 16 mg/kg. When administered at specified dosages and schedules, the combination of ADC and Rad51 inhibitor is said to reduce solid tumors in size, reduce or eliminate metastases and is effective to treat cancers resistant to standard therapies, such as radiation therapy, chemotherapy or immunotherapy. Surprisingly, the combination is said to be effective to treat cancers that are refractory to or relapsed from irinotecan or topotecan.

U.S. Patent Application No. 20130344509 and U.S. Pat. No. 10,202,461, filed by Nakamura, et al., entitled, “Anti-human Trop-2 antibody having an antitumor activity in vivo”, and “Anti-human TROP-2 antibody having an antitumor activity in vivo”, respectively. These inventors are said to teach an antibody that specifically binds to hTROP-2 and has anti-tumor activity in vivo (e.g., a humanized antibody) and a hybridoma that produces the antibody; and a conjugate of the antibody with a drug; a pharmaceutical composition for diagnosing or treating a tumor; a method for detecting a tumor; and a kit for detecting or diagnosing a tumor.

Another such antibody is taught in U.S. Pat. No. 10,501,555, filed by Guerra and Severio, entitled, “Disease Therapy By Inducing Immune Response To Trop-2 Expressing Cells”. These inventors are said to teach humanized anti-Trop-2 antibodies and their fragments, derivatives and conjugates that are able to recognize and bind with high affinity distinct regions of the Trop-2 molecule. These inventors are also said to teaches the use of such antibodies and of pharmaceutical compositions thereof for diagnosis and therapy of human pathologies such as cancer.

Another such antibody is taught in U.S. Pat. No. 9,670,286, filed by Chang, et al., entitled, “Disease Therapy By Inducing Immune Response To Trop-2 Expressing Cells”. These inventors are said to teach bispecific antibodies with at least one binding site for Trop-2 (EGP-1) and at least one binding site for CD3. The bispecific antibodies are said to be used for inducing an immune response against a Trop-2 expressing tumor, such as carcinoma of the esophagus, pancreas, lung, stomach, colon, rectum, urinary bladder, breast, ovary, uterus, kidney or prostate. The bispecific antibody can be used alone or in combination with one or more therapeutic agents such as antibody-drug conjugates, interferons, and/or checkpoint inhibitor antibodies. The bispecific antibody is said to be capable of targeting effector T cells, NK cells, monocytes, or neutrophils to induce leukocyte-mediated cytotoxicity of Trop-2⁺ cancer cells. The cytotoxic immune response is enhanced by co-administration of interferon, checkpoint inhibitor antibody and/or ADC.

Other patents and applications that are said to teach anti-TROP2 antibodies include: U.S. Pat. Nos. 7,420,040 B2; 7,420,041 B2; 10,501,555 B2; 10,202,461 B2; 9,850,312 B2; US 2016/0053018 A1; U.S. Pat. No. 9,399,074 B2; US 2012/0237518 A1; and U.S. Pat. No. 9,770,517 B2.

Despite these advances, a need remains for novel antibodies and bi-or multi-specific anti-TROP2 antibodies that do not have the side effects of the known anti-TROP2 antibodies.

### SUMMARY OF THE INVENTION

As embodied and broadly described herein, an aspect of the present disclosure relates to an anti-Trop 2 antibody or binding fragment thereof, wherein the antibody comprises: a heavy chain variable domain (VH) complementarity determining region (CDR) 1 comprising the amino acid sequence of any one of the following SEQ ID NOs: 2, 12, 22, 32, 42, 52, 62, 72, 82, 92, 102, 112, 122, or 132; and a VH CDR2 comprising the amino acid sequence of any one of the following SEQ ID NOs: 3, 13, 23, 33, 43, 53, 63, 73, 83, 93, 103, 113, 123, or 133; and a VH CDR3 comprising the amino acid sequence of any one of the following SEQ ID NOs: 4, 14, 24, 34, 44, 54, 64, 74, 84, 94, 104, 114, 124, 134; and a light chain variable domain (VL) CDR1 comprising the amino acid sequence of any one of the following SEQ ID NOs: 7, 17, 27, 37, 47, 57, 67, 77, 87, 97, 107, 117, 127, or 137; and a VL CDR2 comprising the amino acid sequence of any one of the following SEQ ID NOs: 8, 18, 28, 38, 48, 58, 68, 78, 88, 98, 108, 118, 128, or 138; and a VL CDR3 comprising the amino acid sequence of any one of the following SEQ ID NOs: 9, 19, 29, 39, 49, 59, 69, 79, 89, 99, 109, 119, 129, or 139. In one aspect, wherein the antibody comprises: the VH comprising the amino acid sequence of any one of the following SEQ ID NOs: 1, 11, 21, 31, 41, 51, 61, 71, 81, 91, 101, 111, 121, 131, 156, 158, 160, or 162, and the VL comprising the amino acid sequence of any one of the following SEQ ID NOs: 6, 16, 26, 36, 46, 56, 66, 76, 86, 96, 106, 116, 126, 136, 164, 166, or 168. In another aspect, the antibody comprises: the VH is encoded by a nucleic acid sequence comprising of any one of the following SEQ ID NOs: 5, 15, 25, 35, 45, 55, 65, 75, 85, 95, 105, 115, 125, 135, 157, 159, 161, or 163, and the VL is encoded by a nucleic acid sequence comprising of any one of the following SEQ ID NOs: 10, 20, 30, 40, 50, 60, 70, 80, 90, 100, 110, 120, 130, 140, 165, 167, or 169. In another aspect, the antibody is a monoclonal, bispecific, multivalent, multi-specific, diabody, chimeric, scFv antibody, or fragments thereof. In another aspect, the antibody is a full-length antibody that is afucosylated. In another aspect, the antibody comprises an Fc domain of any one of the following: human IgG1, human IgG2, human IgG3, and human IgG4. In another aspect, the Fc domain is a wild-type, variant, or truncated Fc domain. In another aspect, the antibody or binding fragment further comprises a second antigen binding domain that binds to a target other than TROP-2. In another aspect, the second antigen target is an anti-CD3 antibody with a heavy chain selected from SEQ ID NO: 174, or 178; and the light chain is selected from SEQ ID NOS: 176 or 180. In another aspect, the antibody is a bispecific antibody comprising a heavy chain selected from SEQ ID NOS: 190 and 192, or 194 and 196.

As embodied and broadly described herein, an aspect of the present disclosure relates to a method of treating a disease in a subject in need thereof, comprising administering to the subject a therapeutically effective amount of any of the antibodies described hereinabove. In another aspect, the disease is a proliferative disease. In another aspect, the proliferative disease is cancer selected from those that express Trop 2 or a mutant thereof. In another aspect, the subject is human.

As embodied and broadly described herein, an aspect of the present disclosure relates to a bi-valent or multi-valent antibody wherein said antibody comprises: an anti-Trop 2 antibody or binding fragment thereof, wherein the antibody comprises: a heavy chain variable domain (VH) complementarity determining region (CDR) 1 comprising the amino acid sequence of any one of the following SEQ ID NOs: 2, 12, 22, 32, 42, 52, 62, 72, 82, 92, 102, 112, 122, or 132; and a VH CDR2 comprising the amino acid sequence of any one of the following SEQ ID NOs: 3, 13, 23, 33, 43, 53, 63, 73, 83, 93, 103, 113, 123, or 133; and a VH CDR3 comprising the amino acid sequence of any one of the following SEQ ID NOs: 4, 14, 24, 34, 44, 54, 64, 74, 84, 94, 104, 114, 124, 134; and a light chain variable domain (VL) CDR1 comprising the amino acid sequence of any one of the following SEQ ID NOs: 7, 17, 27, 37, 47, 57, 67, 77, 87, 97, 107, 117, 127, or 137; and a VL CDR2 comprising the amino acid sequence of any one of the following SEQ ID NOs: 8, 18, 28, 38, 48, 58, 68, 78, 88, 98, 108, 118, 128, or 138; and a VL CDR3 comprising the amino acid sequence of any one of the following SEQ ID NOs: 9, 19, 29, 39, 49, 59, 69, 79, 89, 99, 109, 119, 129, or 139; and a second antibody, antigen-binding of the second antibody or fragment thereof; a target-binding protein, a cytokine; a lectin; or a toxin. In one aspect, the second antibody targets an immune effector cell surface receptor selected from at least one of CTLA-4, PD-1, Lag3, S15, B7H3, B7H4, TCR-alpha, TCR-beta, TIM-3, CD3, 41BB or OX40. In another aspect, the antibody is a multi-valent antibody that targets two or more antigens other than Trop 2. In another aspect, the antibody is a bispecific antibody comprising a heavy chain selected from SEQ ID NOS: 190 and 192, or 194 and 196. In another aspect, the anti-Trop 2 antibody comprises: the VH comprising the amino acid sequence of any one of the following SEQ ID NOs: 1, 11, 21, 31, 41, 51, 61, 71, 81, 91, 101, 111, 121, 131, 156, 158, 160, and 162, and the VL comprising the amino acid sequence of any one of the following SEQ ID NOs: 6, 16, 26, 36, 46, 56, 66, 76, 86, 96, 106, 116, 126, 136, 164, 166, or 168. In another aspect, the anti-Trop 2 antibody comprises: the VH is encoded by a nucleic acid sequence comprising of any one of the following SEQ ID NOs: 5, 15, 25, 35, 45, 55, 65, 75, 85, 95, 105, 115, 125, 135, 157, 159, 161, or 163, and the VL is encoded by a nucleic acid sequence comprising of any one of the following SEQ ID NOs: 10, 20, 30, 40, 50, 60, 70, 80, 90, 100, 110, 120, 130, 140, 165, 167, or 169. In another aspect, the antibody is a monoclonal antibody, a full-length antibody, or an antibody fragment. In another aspect, the antibody is fused to an Fc domain of any one of the following: human IgG1, human IgG2, human IgG3, and human IgG4. In another aspect, the target is CD3 and the antibody a heavy chain selected from SEQ ID NO: 174, or 178; and the light chain is selected from SEQ ID NOS: 176 or 180. In another aspect, the target is CD3 and the antibody a heavy chain is encoded by a nucleic acid selected from SEQ ID NO: 175 or 179; and the light chain is encoded by a nucleic acid selected from SEQ ID NOS: 177 or 181. In another aspect, the Fc domain is a wild-type, variant, or truncated Fc domain. In another aspect, the antibody is a full-length antibody that is afucosylated.

As embodied and broadly described herein, an aspect of the present disclosure relates to a method of treating a disease in a subject in need thereof, comprising administering to the subject a therapeutically effective amount of the antibody or bivalent antibody described hereinabove. In another aspect, the disease is a proliferative disorder. In another aspect, the disease is a proliferative disorder selected from a cancer cell that expresses Trop 2 or mutants thereof selected from cancers of the breast, cervix, colorectal, esophagus, gastric, certain lung cancers, squamous cell carcinoma of the oral cavity, ovary, pancreas, prostate, stomach, thyroid, urinary bladder, and uterus. In another aspect, the subject is human.

As embodied and broadly described herein, an aspect of the present disclosure relates to a nucleic acid encoding an antibody, binding fragment thereof, bivalent, or multivalent nucleic acid sequence comprising: a VH is encoded by a nucleic acid sequence comprising of any one of the following SEQ ID NOs: 5, 15, 25, 35, 45, 55, 65, 75, 85, 95, 105, 115, 125, 135, 157, 159, 161, 163; and a VL is encoded by a nucleic acid sequence comprising of any one of the following SEQ ID NOs: 10, 20, 30, 40, 50, 60, 70, 80, 90, 100, 110, 120, 130, 140, 165, 167, 169; or an antibody encoded by a nucleic acid sequence of SEQ ID NOS: 191 and 193, or 195 and 197; or a bispecific antibody encoded by a nucleic acid sequence of SEQ ID NOS: 175 and 177, 179 and 181, 183 and 185, 191 and 193, 195 and 197, or combinations thereof.

As embodied and broadly described herein, an aspect of the present disclosure relates to a vector comprising a nucleic acid comprising: a VH is encoded by a nucleic acid sequence comprising of any one of the following SEQ ID NOs: 5, 15, 25, 35, 45, 55, 65, 75, 85, 95, 105, 115, 125, 135, 157, 159, 161, 163; and a VL is encoded by a nucleic acid sequence comprising of any one of the following SEQ ID NOs: 10, 20, 30, 40, 50, 60, 70, 80, 90, 100, 110, 120, 130, 140, 165, 167, 169; or an antibody encoded by a nucleic acid sequence of SEQ ID NOS: 191 and 193, or 195 and 197; or a bispecific antibody encoded by a nucleic acid sequence of SEQ ID NOS: 175 and 177, 179 and 181, 183 and 185, 191 and 193, 195 and 197, or combinations thereof.

As embodied and broadly described herein, an aspect of the present disclosure relates to a host cell comprising a vector that comprises a nucleic acid comprising: a VH is encoded by a nucleic acid sequence comprising of any one of the following SEQ ID NOs: 5, 15, 25, 35, 45, 55, 65, 75, 85, 95, 105, 115, 125, 135, 157, 159, 161, 163; and a VL is encoded by a nucleic acid sequence comprising of any one of the following SEQ ID NOs: 10, 20, 30, 40, 50, 60, 70, 80, 90, 100, 110, 120, 130, 140, 165, 167, 169; or an antibody encoded by a nucleic acid sequence of SEQ ID NOS: 187 and 189, or 191 and 193; or a bispecific antibody encoded by a nucleic acid sequence of SEQ ID NOS: 175 and 177, 179 and 181, 183 and 185, 187 and 189, or 191 and 193, or combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present application can be understood by reference to the following description taking in conjunction with the accompanying figures.

FIGS. 1A and 1B show representative sensorgrams of the antibodies of the present invention. Binding kinetics were measured using human Fc-capture on the Carterra LSA high-throughput SPR instrument (Carterra, Inc.). No antibody tested bound to human EpCAM, and nearly all bound equivalently to cynomolgus/rhesus TROP2 (cyTROP2). While none of mouse chimeras recognized mouse TROP2 (msTROP2), nearly all the chicken chimeras displayed an affinity to msTROP2 within 10-fold of the binding to huTROP2.

FIGS. 2A and 2B are graphs that shows MSD binding to TROP2. FIG. 2A. Binding of anti-TROP2 antibodies in a dose-dependent manner to immobilized TROP2 was evaluated using an MSD assay. Several clones displayed EC₅₀ values lower than that of RS7, indicating greater binding potency. ND: Not determined. FIG. 2B. Binding of anti-TROP2 antibodies in a dose-dependent manner to immobilized TROP2 was evaluated using an MSD assay. Several clones displayed EC50 values lower than that of RS7, indicating greater binding potency. ND: Not determined.

FIGS. 3A and 3B show epitope binning of the antibodies taught herein. FIG. 3A. Network plot shown in FIG. 3A showing pairwise competition between a library of 141 antibodies against TROP2 by SPR. Each node represents an individual antibody, and the connecting lines indicated a blocking relationship between the two antibodies. Antibodies with similar blocking profiles, indicating a high probability of having a shared epitope, are organized into 5 groups. FIG. 3B shows the various regions of TROP2. Group 1 contains the majority of the antibodies assessed, including the approved antibody RS7, and likely corresponds to the subset of antibodies that target the immunodominant domain of TROP2. While antibodies in Group 2 form their own cluster, they likely target an epitope at or near the epitopes targeted in Group 1, based on the extent of their interaction with antibodies of that group. Based on peptide mapping data, antibodies in Group 3 likely bind at or near the N-terminal domain while antibodies in Group 4 may bind the membrane proximal domain near to but distinct from the RS7 binding site. The exact epitope targeted by Group 5 remains unclear. FIG. 3B summarizes the bin of each antibody.

FIG. 4 shows the peptide mapping of the antibodies by bin group. Sequence map showing the location of 8 peptides bound by anti-TROP2 antibodies according to SPR data. Not every antibody tested recognized a linear epitope in this assay. Some antibodies within bin Group 3 recognized Peptides 1-2, suggesting an epitope somewhere near the N-terminus of the TROP2 protein. A subset of antibodies that bin together in Groups 1 and 2 recognized Peptides 3-6 in the C-terminal domain near to the putative RS7 epitope, suggesting that those antibodies in those bins target an area at or near those sites. Lastly, a subset of antibodies in Group 4 recognized Peptides 7-8 and thus the group may broadly target the C-terminal region of the C-terminal domain, just proximal to the transmembrane domain and separate from the RS7 epitope.

FIGS. 5A and 5B are graph that shows the results from the ADCC reporter assay for the hits of the mouse antibodies taught herein. FIG. 5A. Dose-response graph shows the ADCC potency of Anti-human Trop2 antibodies in human head and neck cancer cell line FaDu. NFAT CD16 Jurkat ADCC reporter cell lines is used to show ADCC potency by Anti-human Trop2 antibodies and afucosylated anti-human Trop2 antibodies. All afucosylated anti-human Trop2 antibodies show much smaller EC₅₀ value in compared to their fucosylated version, indicating that afucosylated anti-human Trop2 antibodies enhance their ADCC potency. EC₅₀ values are averages of n=2-3 experiments. FIG. 5B. Table showing ADCC potency of Anti-human Trop2 antibodies in FaDu cancer cell line.

FIGS. 6A and 6B is a graph that shows the results from the ADCC reporter assay for hits of the chicken antibodies taught herein. FIG. 6A is a graph that shows ADCC potency of Anti-human Trop2 antibodies in FaDu cancer cell line. Dose-response graph shows the ADCC potency of Anti-human Trop2 antibodies in FaDu cancer cell line. NFAT CD16 Jurkat ADCC reporter cell lines is used to show ADCC potency by Anti-human Trop2 antibodies. Most of the clone shows very similar ADCC potency in compared to RS7. EC₅₀ values are averages of n=1-3 experiments. ADCC potency of Anti-human Trop2 antibodies in Fadu cancer cell line. FIG. 6B. Table which shows that most of the clone shows very similar ADCC potency in compared to RS7. EC50 values are averages of n=1-3 experiments.

FIGS. 7A and 7B show cell binding results for the antibodies taught herein. Anti-human Trop2 antibodies binding potency to human head and neck cancer cell line FaDu. FACS analysis shows in FIG. 7A anti-Trop2 antibodies and RS7 binding to FaDu cells that endogenously expresses human Trop2. Values plotted are Median Fluorescent Intensity. EC₅₀ values in FIG. 7B are averages of n=1-3 experiments.

FIG. 8 is a graph that shows the results from anti-human Trop2 antibodies derived from immunized chickens binding to ExpiCHO cells overexpressing human Trop2. FACS analysis showing anti-Trop2 antibodies and RS7 binding to ExpiCHO cells overexpressing human Trop2 in a dose-dependent manner. Values plotted are percent of antibody binding to target cells. Top dose (66.67 nM) in binding assay is used for histogram overlay comparing antibody binding levels to human Trop2 for the antibodies tested.

FIG. 9 is a graph that shows the results from anti-human Trop2 antibodies derived from immunized chickens binding to ExpiCHO cells overexpressing mouse Trop2. FACS analysis showing select anti-Trop2 antibodies binding to ExpiCHO cells overexpressing mouse Trop2 in a dose-dependent manner, indicating cross-reactivity with mouse Trop2. RS7 control, which is specific to human Trop2 only, does not show binding to ExpiCHO cells overexpressing mouse Trop2. Values plotted are percent of antibody binding to target cells. Top dose (66.67 nM) in binding assay is used for histogram overlay comparing antibody binding levels to mouse Trop2 for the antibodies tested.

FIGS. 10A and 10B show peripheral blood mononuclear cell (PBMC) killing with the anti-human Trop2 antibodies of the present invention, which exhibit strong human ovarian cancer (Ovcar3) cell killing. The graph in FIG. 10A shows FACS analysis of cell death triggered by anti-TROP2 molecules against TROP2 expressing Ovcar3 target cells and human PBMC as effector cells. FIG. 10B contains EC₅₀ values for each tested antibody.

FIG. 11 is an illustration of the study design for in vivo effectiveness.

FIGS. 12A to 12C are graphs that show the results from the in vivo study using FaDu xenograft in nude mice. Randomization and grouping was performed on Day 0, based on tumor size. Dosing was performed on Days 0, 3, 7, 10, and 14 (FIG. 12A) Tumor volume for FaDu nude mice xenograft model. (FIG. 12B) Tumor volume percent change. (FIG. 12C) Mouse body weight.

FIG. 13 shows Humanization of SD-589775. Binding kinetics to huTROP2 were measured using human Fc-capture on the Carterra LSA high-throughput SPR instrument (Carterra, Inc.). Combinations of humanized VH and VL sequences were tested and compared to the parental chimera. No variants with 589-VH_4 or 589-VL_3 showed measurable binding to huTROP2. 589-VH_5 and 589-VL_4 refer to the parental sequences.

FIG. 14 shows ELISA data showing binding of anti-CD3 antibodies to CD3ϵδ heterodimer. Plate coated with antibody, and then detected by dose response of CD3ϵδ-HRP.

FIGS. 15A and 15B show TROP2×CD3 TAA binding arm bispecific screening. Cell killing via coculture of PBMCs and FaDu tumor cells. % cell lysis measured after 48 hours. FIG. 15A shows mouse-derived TROP2 binding antibodies (as well as SD-589775). FIG. 15B shows chicken-derived TROP2 binding antibodies.

FIGS. 16A to 16C show TROP2×CD3 bispecific cell killing characterization against FaDu. FIG. 16A shows cell killing via coculture of PBMCs and FaDu tumor cells. % cell lysis measured after 48 hours. FIG. 16B shows T cell activation induced by coculture of PBMCs with FaDu tumor cells. T cell activation measured after 48 hours. FIG. 16C shows cytokine production and release induced by coculture of PBMCs with FaDu tumor cells. Cytokine levels measured after 24 hours.

FIGS. 17A to 17C show TROP2×CD3 bispecific cell killing characterization against Calu-3. FIG. 17A shows cell killing via coculture of PBMCs and Calu-3 tumor cells. % cell lysis measured after 48 hours. FIG. 17B shows T cell activation induced by coculture of PBMCs with Calu-3 tumor cells. T cell activation measured after 48 hours. FIG. 17C shows cytokine production and release induced by coculture of PBMCs with Calu-3 tumor cells. Cytokine levels measured after 24 hours.

FIGS. 18A to 18C show TROP2×CD3 bispecific cell killing characterization against OvCar-3. FIG. 18A shows cell killing via coculture of PBMCs and OvCar-3 tumor cells. % cell lysis measured after 48 hours. FIG. 18B shows T cell activation induced by coculture of PBMCs with OvCar-3 tumor cells. T cell activation measured after 48 h. FIG. 18C shows cytokine production and release induced by coculture of PBMCs with OvCar-3 tumor cells. Cytokine levels measured after 24 hours.

FIG. 19 is a graph that shows the results of an in vivo efficacy study using multiple doses of SD-174078 FaDu xenograft in humanized NSG MHC I/II dKO mice. Mice were engrafted with 20×10\hat{ }6 human PBMCs at day −14 via intravenous injection. At day −1, 2×10\hat{ }6 FaDu cells were engrafted subcutaneously. At days 0, 3, and 7, mice were dosed with either PBS (vehicle) or SD-174078 (1 mg/kg) via intravenous injection. Tumor volume is shown. Significance was determined using Welch's T-test.

FIG. 20 is a graph that shows the results of an in vivo efficacy study using a single dose of SD-231831. FaDu xenograft in humanized NSG MHC I/II dKO mice. Mice were engrafted with 20×10\hat{ }6 human PBMCs at day −14 via intravenous injection. At day −1, 2×10\hat{ }6 FaDu cells were engrafted subcutaneously. At day 7, mice were given a single dose of either PBS (vehicle) or SD-231831 (1 mg/kg) via intravenous injection. Tumor volume is shown. Significance was determined using Welch's T-test.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

TROP2 is pan tumor transmembrane glycoprotein that is overexpressed in many human epithelial cancers, with positive correlation between expression and tumor growth, as well as metastasis. TROP2 has 4 N-glycosylation sites, with aberrant glycosylation in some tumors. TROP2 is an emerging target in cancer therapy, with growing number of ADCs in clinical and preclinical development (including one approved therapeutic, Trodelvy). The present invention includes novel anti-TROP2 antibodies.

The term “antibody” as used herein throughout is used in the broadest sense and includes a monoclonal antibody, polyclonal antibody, human antibody, humanized antibody, non-human antibody, chimeric antibody, a monovalent antibody, an antibody fragment, and a tandem scFv-Fc antibody.

Antibody fragments of the disclosure retain antigen binding specificity. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as retaining antigen binding specificity.

The term “meso-scale engineered molecule (MEM)” as used herein throughout includes engineered peptides and polypeptides between about 1 kDa and about 10 kDa. The term “MEM-nanoparticle” as used herein throughout includes MEMs which have been conjugated to a nanoparticle (e.g., ferritin nanoparticle).

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

All publications, patents, and patent applications mentioned in this specification are herein incorporated by reference to the same extent as if each individual publication, patent, or patent application was specifically and individually indicated to be incorporated by reference.

### Antibodies

As used herein, the term “antibody” refers to an intact antibody or a binding fragment thereof that binds specifically to a target antigen. Binding fragments are produced by recombinant DNA techniques, or by enzymatic or chemical cleavage of intact antibodies. Binding fragments include Fab, Fab′, F(ab′)₂, Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)₂, Fv, scFv) so long as they exhibit the desired biological activity. Antibodies (Abs) and immunoglobulins (Igs) are glycoproteins having the same structural characteristics. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including plant cells.

As used herein, the term “antibody fragment” refers to a portion of a full-length antibody, generally the antigen-binding or variable region, and include Fab, Fab′, F(ab′)₂, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)₂ fragment that has two antigen-binding fragments which are capable of cross-linking antigen, and a residual other fragment (which is termed pFc′). As used herein, “functional fragment” with respect to antibodies, refers to Fv, F(ab) and F(ab′)₂ fragments.

As used herein, the “Fv” fragment is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (V_{H}) followed by the constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, et al. (1989), Nature 342: 877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.) The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for TROP2 is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fr framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The presently disclosed and claimed invention further includes the use of fully human monoclonal antibodies cross-reactive against Trop 2. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425;5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the term “treatment” refers to both therapeutic treatment and prophylactic or preventative measures. Those in need of treatment include those already with the disorder as well as those in which the disorder is to be prevented.

As used herein, the term “disorder” refers to any condition that would benefit from treatment with the polypeptide. This includes chronic and acute disorders or diseases including those infectious or pathological conditions that predispose the mammal to the disorder in question.

An antibody or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in W0/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

The antibody can be designed to alter the effector function, e.g., by binding to effector molecules of the CD3 complex and thereby changing the T cell-mediated cytotoxicity (TDCC) activity. These “effector functions” are responsible for activating or diminishing a biological activity (e.g., in a subject). Examples of effector functions include, but are not limited to: Such effector functions can be assessed using various assays (e.g., cytokine release assays T cell proliferation, T_{H} or T_{C} activity, etc.).

In some embodiments, bispecific or multispecific antibodies are provided, which comprise at least a heavy chain variable region from the antibody family of the invention and may comprise a heavy and light chain variable region provided herein. Bispecific antibodies comprise at least the heavy chain variable region of an antibody specific for TROP2 a protein other than TROP2, and may comprise a heavy and light chain variable region. Various formats of bispecific antibodies are within the scope of the invention, including without limitation single chain polypeptides, two chain polypeptides, three chain polypeptides, four chain polypeptides, and multiples thereof.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

The Fc region can be a wild-type Fc region, a mutated Fc region, a monomeric wild-type Fc region, a monomeric mutant Fc region, a dimeric wild-type Fc region, or a dimeric mutant Fc region, a second variable heavy region and a second Fc region, or a second variable heavy region and a second Fc region, and may further include an uncleavable flexible linker and a second variable light region and a second heavy variable region, or a second Fc region and an uncleavable flexible linker and payload, such as a toxin, a cytokine, or another antibody. Non-limiting examples of Fc mutants include those such as knob-hole variants that allow for the directed formation of bispecific or multivalent antibodies.

For example, one can generate a variant Fc region of an antibody with improved Clq binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix-and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a carbohydrate, small molecule, lipid, etc.) that when present in the proper conformation, provides a reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

As used herein, the term “treatment” refers to any of (i) the prevention of infection or reinfection, as in a traditional vaccine, (ii) the reduction or elimination of symptoms, and (iii) the substantial or complete elimination of the cells with TROP2 in question. Treatment may be effected prophylactically (prior to the cells becoming cancerous and/or metastatic) or therapeutically (following the cells becoming cancerous and/or metastatic).

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In some embodiments, the target-binding polypeptide binds to a tumor targeting antigen selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4(EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein.

In some embodiments, the target-binding polypeptide binds to a T-cell marker selected from CTLA-4, PD-1, Lag3, S15, B7H3, B7H4, TCR-alpha, TCR-beta, or TIM-3, CD3, 41BB or OX40.

In some embodiments, the target-binding polypeptide binds to an antigen-presenting cell marker selected from PD-L1, CD40, CD24, B7H3, TGF-beta receptor, TNFR family members 1 to 20, CD80, CD86, FLT3, CD11c, CD8-alpha, 5B6 (CLEC9A), CD1c, CD11b, CD13, CD33, HLA-DR, CD141, CD1a, CD32, CD45, CD80, CD86, CD207, CD2, CD7, CD45RA, CD68, CD123, CD303, CD304.

The anti-Trop 2 antibodies or a binding portion (or fragments thereof) can be used to target cancer cells, such as cancer cells that express Trop 2 or mutants thereof selected from cancers of the breast, cervix, colorectal, esophagus, gastric, certain lung cancers, squamous cell carcinoma of the oral cavity, ovary, pancreas, prostate, stomach, thyroid, urinary bladder, and uterus.

### Example 1

Immunization of animals. Mice were immunized using a combination of huTROP2-huFc, huTROP2-His, SK-BR-3 cells, and MCF7 cells 6 total times over the course of 7 weeks, with Incomplete Freund's Adjuvant used for immunization with protein and Sigma Adjuvant System used for cell immunizations. All immunizations were done via intraperitoneal injection. Spleen and lymph nodes were collected after 7 weeks, and cell suspensions prepared. RNA was isolated from single cell suspensions using the Direct-zol RNA miniprep kit (Zymo Research, Cat #R2050), and concentration and purity determined by measured Abs260, Abs280, and Abs230. Quality was confirmed via micro-capillary electrophoresis using a 2100 Bioanalyzer (Agilent Technologies).

Chickens were immunized using a combination of huTROP2-huFc, msTROP2-His, and SK-BR-3 cells 5 total times over the course of 12 weeks, with Complete Freund's Adjuvant and Incomplete Freund's Adjuvant used as adjuvants for huTROP2-huFc immunizations. All immunizations were done via subcutaneous injection. Spleen and bone marrow were collected after 12 weeks, and cell suspensions prepared. RNA was isolated from single cell suspensions using the Direct-zol RNA miniprep kit (Zymo Research, Cat #R2050), and concentration and purity determined by measured Abs260, Abs280, and Abs230. Quality was confirmed via micro-capillary electrophoresis using a 2100 Bioanalyzer (Agilent Technologies).

Preparation of phage display library. From each mouse RNA prep, cDNA was generated using the SuperSCript III First-Strand cDNA kit (Invitrogen, Cat #18080-51). The VH region was amplified using primers binding in the mouse VH framework 1 region and hinge regions, and the VL region amplified using primers binding in the mouse VL kappa framework 1 region and kappa constant region. Expand High Fidelity enzyme (Roche, Cat #0473876001) was used for amplifications. Nested PCR was then performed on each amplified sample to add restriction sites to enable cloning of the VH and VL sequences for phage display libraries.

From each chicken RNA prep, cDNA was generated using the SuperSCript III First-Strand cDNA kit (Invitrogen, Cat #18080-51). The VH region was amplified using primers binding in the chicken VH framework 1 region and VH framework 4 region, and the VL region amplified using primers binding in the chicken VL lambda framework 1 region and lambda constant region. Expand High Fidelity enzyme (Roche, Cat #0473876001) was used for amplifications. Nested PCR was then performed on each amplified sample to add restriction sites to enable cloning of the VH and VL sequences for phage display libraries.

VH and VL sequences were sequentially digested with appropriate restriction enzymes, and ligated into digested phagemid vector for FAB phage display. Ligations were transformed into ECC TG1 cells (Lucigen, Cat #60502-2), and library quality determined by size and VH/VL insert percentage. In each case, a minimum library size was 5×10\hat{ }7 with an insert percentage of 85%; all libraries exceeded these criteria.

Phage display screening. Mouse library phage display selection was performed using both soluble protein antigen and TROP2 expressing cells. In-solution selections were performed using biotinylated huTROP2, with Sera-Mag SPeedBeads Neutravidin—Coated Magnetic Particles (GE Healthcare, Cat #GE78152104010150) or Sigma Dynabeads MyOne Streptavidin T1 Magnetic Beads (Invitrogen, Cat #65602) for selection on a KingFisher Flex. Antigen was used at a range of concentrations, and an off-rate wash using non-biotinylated huTROP2 was performed as a final selection in some cases. Elution was done with Trypsin (Sigma, Cat #T1426), and selection buffer was skimmed milk in 1×PBS. Cell selections were done using SK-BR-3, CHO-K1 overexpressing huTROP2, and 293FF overexpressing huTROP2. For cell selections, 10% FBS in 1×PBS was used as selection buffer, and Trypsin used for elution.

After panning, single clones were isolated and sequenced. VH sequences were then cloned into huIgG1 expression plasmid, and VL sequences were cloned into either huVLkappa or huVLlambda expression plasmids.

Antibody expression and expression. Antibody expression plasmids were transiently introduced into an animal cell line using the ExpiFectamine CHO Transfection Kit (ThermoFisher, Cat #A29129) to yield transfectants that produced anti-TROP2 chimeric or humanized antibody. For a host cell line, ExpiCHO-S (ThermoFisher, Cat #A29127) or a suspension CHO cell line with the α1,6 fucosyltransferase (FUT8) gene knocked out were used (referred to as “WT CHO” and “FUT8 CHO” in further references). After 6-12 days of growth post introduction of DNA, cell suspensions of WT CHO or FUT8 CHO were harvested via centrifugation for 20 min at 4,000×g, and then filtered using 0.2 μm Disposable PES Filter units (FisherScientific, Cat #FB12566504). Anti-TROP2 antibody was recovered from filtrate using Protein A purification (HiTrap MabSelect SuRe, Cytiva Cat #GE11-0034-93). WT CHO was used to express antibodies with standard glycosylation, and FUT8CHO used to express afucosylated antibodies with enhanced effector function (indicated by “-afuc”).

Cell Binding Assay. FaDu cells (ATCC, Cat #HTB-43) were cultured in EMEM (ATCC, Cat #30-2003) supplemented with 10% FBS (Sigma, Cat #F5135) and 1× Penicillin- Streptomycin (Corning, Cat #30-002-CI). ExpiCHO-S cells (ThermoFisher, Cat #A29127) were cultured in ExpiCHO expression medium (Gibco, Cat #A29100-01).

For the cell binding assay, PBS (Corning, Cat #21-040-CV) supplemented with 2% FBS and 2 mM EDTA (Quality Biological, Cat #351-027-721) was used as the assay buffer. Cells were counted then resuspended in the assay buffer, then seeded onto 96-well plates (VWR, Cat #89089-826) at 0.5 or 1×10⁵ cells/well. The plates were centrifuged and then kept on ice for the remaining essay steps after removal of the supernatant. Following supernatant removal, the indicated antibodies were diluted in the assay buffer and added to the cells at increasing concentrations (0.64-10000 ng/mL) for 20 minutes on ice. After incubation, the plates were centrifuged and the supernatant was removed, then the cells were washed once with the assay buffer followed by centrifugation and wash removal. After wash, rat-anti-human IgG Fc Alexa Fluor 647 (Biolegend, Cat #410714) or rabbit-anti-goat IgG Alexa Fluor 647 secondary antibody (JIR, Cat #305-605-046) was added to the cells at 1:200 or 1:800 dilution in the assay buffer, respectively, for 20 minutes on ice. After incubation, the plates were centrifuged and the supernatant was removed, then the cells were washed once with the assay buffer followed by centrifugation and wash removal. After wash, DAPI (Biolegend, Cat #422801) was added to the cells at 1:5000 dilution in the assay buffer. Cell binding was analyzed on a Miltenyi MACSQuant 16 flow cytometer. Flow cytometry data were analyzed with the FlowJo flow cytometry analysis software. For making graphs and calculation of the EC₅₀ values GraphPad Prism 9.3.0 was used.

Binding kinetics. Binding of antibodies to human, mouse, and cynomolgus/rhesus TROP2 and human EpCAM was assessed by surface plasmon resonance (SPR) using the Carterra LSA (Carterra, Inc.). An anti-human IgG capture lawn was first prepared on an HC30M chip (Carterra, Cat #4279) by primary amine coupling. Briefly, the chip surface was activated for 10 minutes with a mixture of 133 mM EDC (ThermoFisher, Cat #22980) and 33.3 mM sulfo-NHS (Thermo Fisher, Cat #24525) in 100 mM MES pH 5.5 (Carterra, Cat #3625), after which the goat anti-human IgG (Southern Biotech, Cat #2040-01) was coupled for 15 minutes at 50 g/mL in 10 mM sodium acetate buffer at pH 4.5 (Carterra, Cat #3622). The unconjugated space on the chip surface was blocked with 1 M ethanolamine HCL pH 8.5 (Carterra, Cat #3626) for 7 minutes. For capture kinetics, the prepared anti-human IgG surface and the 96 channel printhead (96PH) was used to capture an antibody panel for 10 minutes at 1-10 μg/mL in HBSTE buffer (10 mM HEPES pH 7.4, 150 mM NaCl, 3 mM EDTA, 0.05% Tween 20; Carterra, Cat #3630). Purified recombinant antigens (human TROP2, Acro Biosystems, Cat #TR2-H5223; mouse TROP2, Cat #TR2-M52H6; cyno/rhesus TROP2, Cat #TR2-R52H3; human EpCAM, Cat #EPM-H5223) were then injected using the single flow cell (SFC) over the antibody panel at 5 concentrations in a 5-fold dilution series, beginning at 500 nM for human TROP2 and 1 μM for others. Each injection used a 5-minute association phase and a 15-minute dissociation phase. The surface was regenerated between antigens with 0.425% H₃PO₄ (Carterra, Cat #3637). The running buffer for antigen injections was HBSTE supplemented with 0.5 mg/mL BSA (VWR, Cat #97061-422). Binding data was double referenced by subtracting an interspot reference response as well as buffer-only blank responses. The resulting sensorgrams were globally fit to a 1:1 Langmuir binding model to estimate the association rate constant (kₐ), dissociation rate constant (k_{d}), and the dissociation constant (K_{D}) using the Carterra Kinetics software.

Table 1 shows the binding kinetics were measured using human Fc-capture on the Carterra LSA high-throughput SPR instrument (Carterra, Inc.). Mouse/human chimeric anti-TROP2 chimeric antibodies isolated from immunized mice bind to human TROP2 with low-nanomolar/high-picomolar affinities. Chicken/human chimeric antibodies also bound to human TROP2 with low-nanomolar/high-picomolar affinities Many showed equal or greater affinity to TROP2 than RS7. Some clones also displayed non-1:1 kinetic profiles.

TABLE
SPR binding to TROP2

| Clone | Origin | ka (M⁻¹ s⁻¹) | kd (s⁻¹) | KD (nM) |
|---|---|---|---|---|
| RS7 | Mouse | 1.11 × 10⁵ | 2.01 × 10⁻⁴ | 1.81 |
| SD-025096 | Mouse | 3.01 × 10⁵ | 1.65 × 10⁻³ | 5.47 |
| SD-661765 | Mouse | 3.48 × 10⁵ | 5.74 × 10⁻⁴ | 1.65 |
| SD-175533 | Mouse | 1.97 × 10⁵ | 3.96 × 10⁻⁵ | 0.20 |
| SD-813149 | Mouse | 3.77 × 10⁵ | 1.63 × 10⁻⁴ | 0.43 |
| SD-589775 | Chicken | 6.43 × 10⁵ | 2.23 × 10⁻⁴ | 0.35 |
| SD-530939 | Chicken | 3.37 × 10⁵ | 1.55 × 10⁻⁴ | 0.46 |
| SD-802432 | Chicken | 5.42 × 10⁵ | 5.53 × 10⁻⁵ | 0.10 |
| SD-168804 | Chicken | 2.55 × 10⁴ | 2.94 × 10⁻⁴ | 11.5 |
| SD-071877 | Chicken | 4.57 × 10⁵ | 1.43 × 10⁻⁴ | 0.31 |
| SD-457798 | Chicken | 4.03 × 10⁵ | 2.59 × 10⁻⁴ | 0.64 |
| SD-394517 | Chicken | 3.96 × 10⁵ | 3.03 × 10⁻⁴ | 0.76 |
| SD-970200 | Chicken | 5.55 × 10⁵ | 1.14 × 10⁻⁴ | 0.21 |
| SD-320063 | Chicken | 1.48 × 10⁵ | 3.42 × 10⁻⁵ | 0.23 |
| SD-685208 | Chicken | 2.35 × 10⁵ | 8.98 × 10⁻⁴ | 3.81 |

Table 2 shows the binding kinetics were measured using human Fc-capture on the Carterra LSA high-throughput SPR instrument (Carterra, Inc.). No antibody tested bound to human EpCAM, and nearly all bound equivalently to cynomolgus/rhesus TROP2 (cyTROP2). While none of mouse chimeras recognized mouse TROP2 (msTROP2), nearly all the chicken chimeras displayed an affinity to msTROP2 within 10-fold of the binding to huTROP2.

TABLE 2
SPR binding to msTROP2, cyTROP2, and EPCAM.

| | | | | | | Fold difference | Fold difference |
|---|---|---|---|---|---|---|---|
| | | KD (nM) | | | | in affinity | in affinity |
| Clone | Origin | huTROP2 | msTROP2 | cyTROP2 | EpCAM | (huTROP2:msTROP2) | (huTROP2:cyTROP2) |
| RS7 | Humanized | 1.81 | ND | 2.05 | ND | — | 1.1 |
| SD-025096 | Mouse | 5.47 | ND | 6.55 | ND | — | 1.2 |
| SD-661765 | Mouse | 1.65 | ND | 1.82 | ND | — | 1.1 |
| SD-175533 | Mouse | 0.20 | ND | 0.10 | ND | — | 0.5 |
| SD-813149 | Mouse | 0.43 | ND | 0.38 | ND | — | 0.9 |
| SD-589775 | Chicken | 0.35 | 0.35 | 0.48 | ND | 1.0 | 1.4 |
| SD-530939 | Chicken | 0.46 | 0.96 | 0.60 | ND | 2.1 | 1.3 |
| SD-802432 | Chicken | 0.10 | 0.30 | 0.13 | ND | 2.9 | 1.3 |
| SD-168804 | Chicken | 11.5 | 13.6 | 15.3 | ND | 1.2 | 1.3 |
| SD-071877 | Chicken | 0.31 | ND | 4.96 | ND | — | 15.8 |
| SD-457798 | Chicken | 0.64 | 0.34 | 0.96 | ND | 0.5 | 1.5 |
| SD-394517 | Chicken | 0.76 | 1.18 | 0.92 | ND | 1.5 | 1.2 |
| SD-970200 | Chicken | 0.21 | 0.63 | 0.29 | ND | 3.0 | 1.4 |
| SD-320063 | Chicken | 0.23 | 1.59 | 0.11 | ND | 6.9 | 0.5 |
| SD-685208 | Chicken | 3.81 | 11.4 | 6.08 | ND | 3.0 | 1.6 |

Epitope binning. Binning of antibodies by epitope similarity was performed with SPR using the Carterra LSA (Carterra, Inc.). Briefly, an antibody ligand array was immobilized on a sensor chip surface, after which TROP2 and a panel of analyte antibodies were injected one-by-one. In total, 141 antibodies were assessed using this one-on-many pairwise approach. Ligand antibodies can either block the binding of the analyte, suggesting a common or close epitope, or sandwich, indicating both antibodies can bind to the antigen simultaneously and thus likely target a separate epitope.

To prepare the ligand array, a mixture of 133 mM EDC (ThermoFisher, Cat #22980) and 33.3 mM sulfo-NHS (ThermoFisher, Cat #24525) in 100 mM MES pH 5.5 (Carterra, Cat #3625) was injected on the whole surface of an HC30M chip (Carterra, Cat #4279) for 8 minutes with the SFC. The 96PH was then used to couple the array. Each ligand antibody was printed for 15 minutes at a final concentration of 1-10 μg/mL in 10 mM sodium acetate buffer at pH 4.5 (Carterra, Cat #3622). The unconjugated space on the chip surface was blocked with 1 M ethanolamine HCL at pH 8.5 (Carterra, Cat #3626) for 7 minutes followed by washes with running buffer (25 mM MES+0.01% Tween 20; Carterra, Cat #3631). Ligand integrity and regeneration conditions were assessed by performing several injections of TROP2 antigen (Acro Biosystems, Cat #TR2-H5223) over the completed surface followed by different regeneration solutions.

To prepare the analytes, each antibody was diluted to 1-10 μg/mL in running buffer (HBSTE+0.5 mg/mL BSA; Carterra, Cat #3630; VWR, Cat #97061-422) and arrayed in a 384 extra-deep-well plate for injection with the SFC. TROP2 antigen was prepared at 100 nM in the running buffer and 10 mM glycine at pH 2.0 (Carterra, Cat #3640) was used for regeneration. Each of the 141 binning cycles consisted of a 1 minute baseline period, 5 minutes of antigen injection, 5 minutes of analyte injection, 2 pulses of regeneration solution for 15 seconds each, and 1 minute of stabilization. An injection of running buffer instead of analyte was performed every 12^{th} cycle. Pairwise binding data were analyzed using the Carterra Epitope software.

Epitope mapping. Binding of antibodies to a peptide array derived from TROP2, allowing for more precise epitope mapping, was evaluated with SPR using the Carterra LSA (Carterra, Inc.). Human TROP2 was first synthesized at 50 nmol scale as an overlapping set of biotinylated 15-residue peptides (with 5-residue overlaps, JPT Peptide Technologies), each containing a C-terminal biotin and N-terminal glycine amide. A total library of 47 peptides was synthesized and each was reconstituted to 1 mg/mL in DMSO and diluted to 2 and 1 μg/mL in HBS (10 mM HEPES pH 7.4, 150 mM NaCl) in a 96-well plate. The peptide array was then printed to the surface of a streptavidin-coated sensor chip (SAHC30M, Carterra, Cat #4294) using the 96PH. Biotinylated TROP2 (Acro Biosystems, Cat #TR2-H82E5) was included in the array as a positive control. The entire array was printed to two blocks for 12 minutes each. A panel of 141 antibodies in HBSTE buffer was then injected one-by-one over the array for 5 minutes, with two 15-second pulses of regeneration solution (0.425% phosphoric acid; Carterra, Cat #3637) between each. Mapping data were analyzed using the Carterra Epitope software.

TROP2 binding assay. Binding of a panel of antibodies against immobilized TROP2 was assessed using an MSD assay. All incubations were carried out for 1 hour at room temperature, and each plate was washed 3 times with Wash buffer (MSD, Cat #R61AA) between each step. MSD GOLD 96-well Streptavidin QUICKPLEX plates (MSD, Cat #L55SA) were first blocked with 150 μL/well MSD Blocker A (MSD, Cat #R93BA). Plates were then coated with 0.1 μg/mL biotinylated TROP2 (Acro Biosystems, Cat #TR2-H82E5) diluted in Diluent 100 (MSD, Cat #R50AA). An 8-point dose-response curve was created for each antibody using 5-fold serial dilutions starting at 10 μg/mL in Diluent 100. After incubation of the antibody samples, bound antibody was detected with a goat anti-human SULFO-TAG antibody (MSD, Cat #R32AJ). Before reading, 150 μL/well of MSD GOLD Read Buffer A (MSD, Cat #R92TG) was added and plates were read on a MESO QuickPlex SQ 120MM instrument. Dose-response curves were fit with non-linear regression and 1/Y² weighting to calculate EC₅₀ using GraphPad Prism.

ADCC Reporter Assay. FaDu cells (ATCC, Cat #HTB-43) were cultured in EMEM (ATCC, Cat #30-2003) supplemented with 10% FBS (Sigma, Cat #F5135) and 1× Penicillin- Streptomycin (Corning, Cat #30-002-CI). For ADCC reporter assay, FaDu cells were counted to assess the cell number and viability. Cells were centrifuged and resuspended in growth media at 5×10⁵ cells/mL. 5×10⁴ cells were seeded per well onto 96 well plates (VWR, Cat #89131-676) and incubated in cell culture incubator overnight. Anti-TROP2 or control antibodies in Xvivo 15 media were added to the cells at increasing concentrations (0.32-5000 ng/mL) for 20 minutes at 37° C., 5% CO₂. After incubation with the antibodies, 1×10⁵ Jurkat NFAT/CD16 cells were added into each well then incubated at 37° C., 5% CO2 for 16-17 hrs. After incubation, the assay plates were equilibrated at room temperature for 15 minutes prior to plate reading. To look at reporter cell activation, 20 μL of media from each well of the assay plate was added into the corresponding well of a flat-bottom white 96-well read plate (VWR, Cat #89130-330), along with 50 μL/well of the QUANTI-Luc Gold (Invivogen, Cat #rep-qlcg1) solution. The plates were read using Promega GloMax plate reader (or Molecular devices ID5) set for luminescence reading with an integration time of 0.5 sec/well. The data was normalized by subtracting the mean baseline luminescence values from the blank wells from all other wells and analyzed using Graphpad Prism 9.3.0, including calculation of the EC₅₀ values.

ADCC/PBMC assay. Ovcar3 cells (ATCC, Cat #HTB-161) were cultured in RPMI (ATCC, Cat #30-2001) supplemented with 20% FBS (Sigma, Cat #F5135) and 1× Penicillin- Streptomycin (Corning, Cat #30-002-CI), 0.01 mg/ml bovine insulin. For ADCC PBMC assay, Ovcar3 cells were counted to assess the cell number and viability. Ovcar3 cells were stained with CFSE. Cells were centrifuged and resuspended in growth media at 1×10⁵ cells/mL seeded at 1×10⁴ cells per well onto 96 well plates (VWR, Cat #89131-676) and incubated in cell culture incubator overnight. Anti-TROP2 or control antibodies in Assay media were added to the cells at increasing concentrations (0.032-500 ng/ml) for 20 minutes at 37° C., 5% CO₂. After that, 2×10⁵ peripheral blood mononuclear cells (PBMC) (Stemcell, Cat #70025.1) were added per well of 96 well plate. Cells and antibodies were incubated at 37° C. in 5% CO₂ incubator for 18-24 hours. Samples were then stained with LIVE/DEAD™ fixable Aqua Dead Cell Stain Kit (Thermo Fisher, Cat #L34957) and analyze using flow cytometer. The % dead target cell was gated for FITC+ Live/dead+. The EC₅₀ value was calculated using Graphpad Prism 9.3.0.

In vivo Efficacy Assay. Seven-week-old female nude mice (Charles River Laboratories, Cat #088Nu/Nu) were used in this assay. 2×10⁶ FaDu cells in 100 μL the mixture of PBS and MatriGel (Corning, Cat #354234) (v:v=1:1) were inoculated to the left upper side of each nude mouse by sub-Q injection. Tumor growth and mouse body weight were monitored twice a week. For each individual tumor, the longest longitudinal diameter as length and the widest transverse diameter as width were measured by using a Traceable Digital Calliper (VWR, Cat #62379-531). Tumor volume (TV) were then calculated by the formula TV=[length×(width)²]/2. Mice were grouped randomly when averaged tumor volume reached 170-175 mm³, and each group contained 10 mice. Human IgG1 SD-025096 was made in a stock solution of 3 mg/mL in PBS. For each mouse, the volume of administered vehicle control (PBS) or antibody SD-025096 was calculated by the formula Volume (μL)=Mouse body weight (g)×10 μL/g. Vehicle control and antibody drug were administered twice a week for 6 times total (biw×6) via the intravenous route. After initial drug treatment, the percentage change of each tumor was calculated by the formula: TV change %=[(TV−TV_{day0})/TV_{day0}]×100.

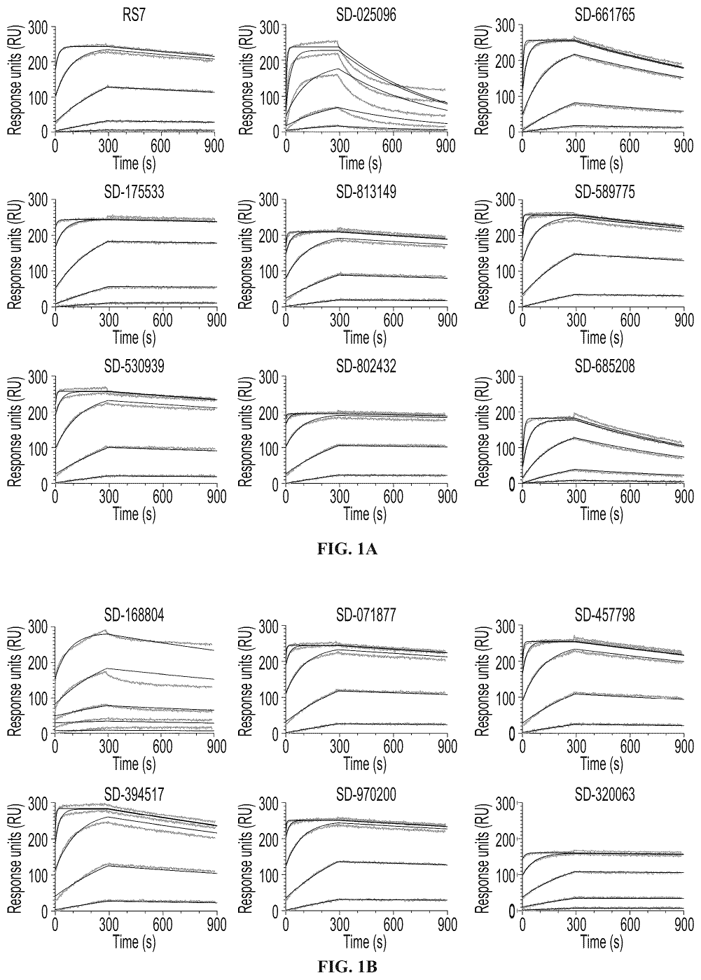

FIGS. 1A and 1B show representative sensorgrams of the antibodies of the present invention. Binding kinetics were measured using human Fc-capture on the Carterra LSA high-throughput SPR instrument (Carterra, Inc.). No antibody tested bound to human EpCAM, and nearly all bound equivalently to cynomolgus/rhesus TROP2 (cyTROP2). While none of mouse chimeras recognized mouse TROP2 (msTROP2), nearly all the chicken chimeras displayed an affinity to msTROP2 within 10-fold of the binding to huTROP2.

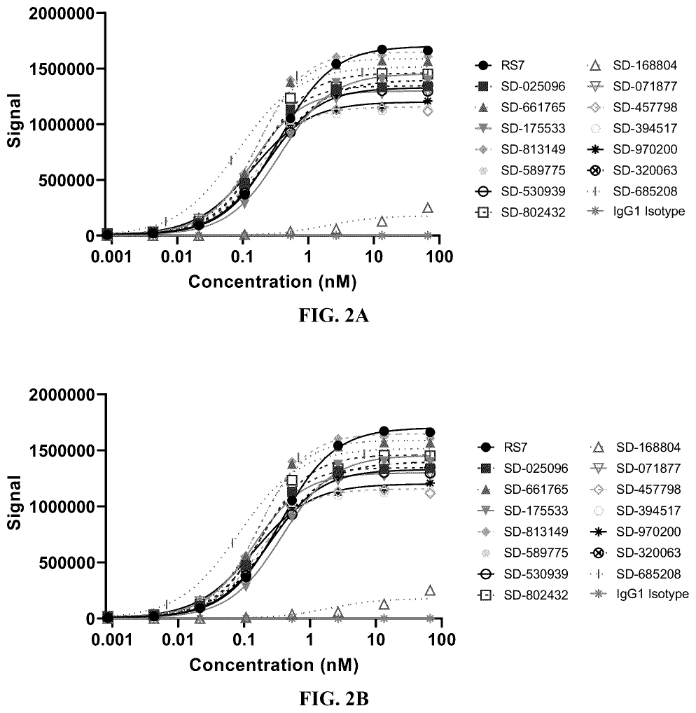

FIGS. 2A and 2B show MSD binding to TROP2. FIG. 2A is a graph that shows binding of anti-TROP2 antibodies in a dose-dependent manner to immobilized TROP2 was evaluated using an MSD assay. FIG. 2B shows derived EC₅₀ values, where several clones displayed EC₅₀ values lower than that of RS7, indicating greater binding potency. ND: Not determined.

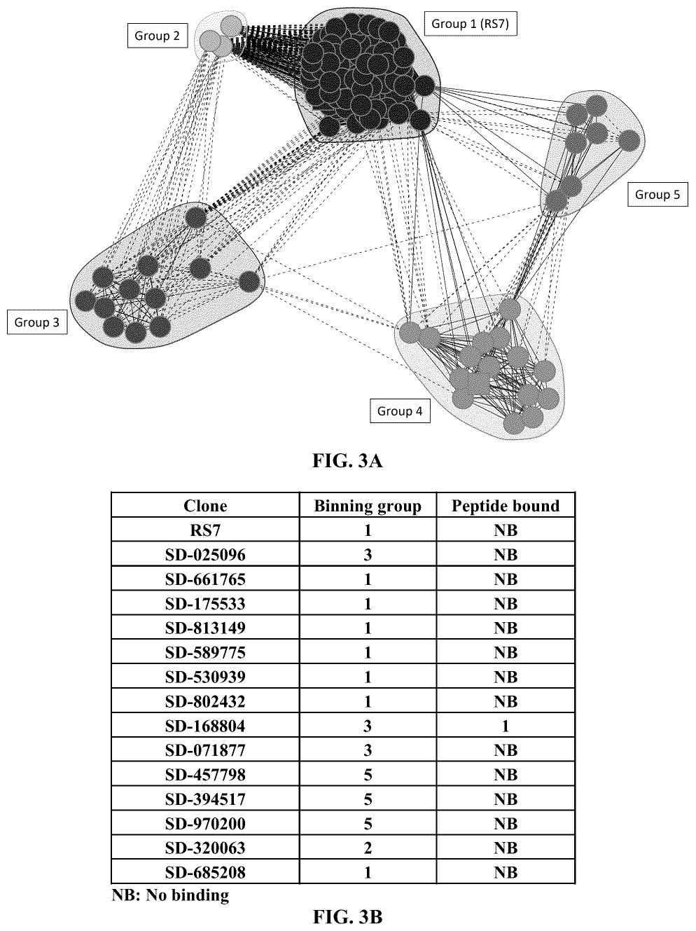

FIGS. 3A and 3B show epitope binning of the antibodies taught herein. FIG. 3A. Network plot shown in FIG. 3A showing pairwise competition between a library of 141 antibodies against TROP2 by SPR. Each node represents an individual antibody, and the connecting lines indicated a blocking relationship between the two antibodies. Antibodies with similar blocking profiles, indicating a high probability of having a shared epitope, are organized into 5 groups. FIG. 3B shows the various regions of TROP2. Group 1 contains the majority of the antibodies assessed, including the approved antibody RS7, and likely corresponds to the subset of antibodies that target the immunodominant domain of TROP2. While antibodies in Group 2 form their own cluster, they likely target an epitope at or near the epitopes targeted in Group 1, based on the extent of their interaction with antibodies of that group. Based on peptide mapping data, antibodies in Group 3 likely bind at or near the N-terminal domain while antibodies in Group 4 may bind the membrane proximal domain near to but distinct from the RS7 binding site. The exact epitope targeted by Group 5 remains unclear. FIG. 3B summarizes the bin of each antibody.

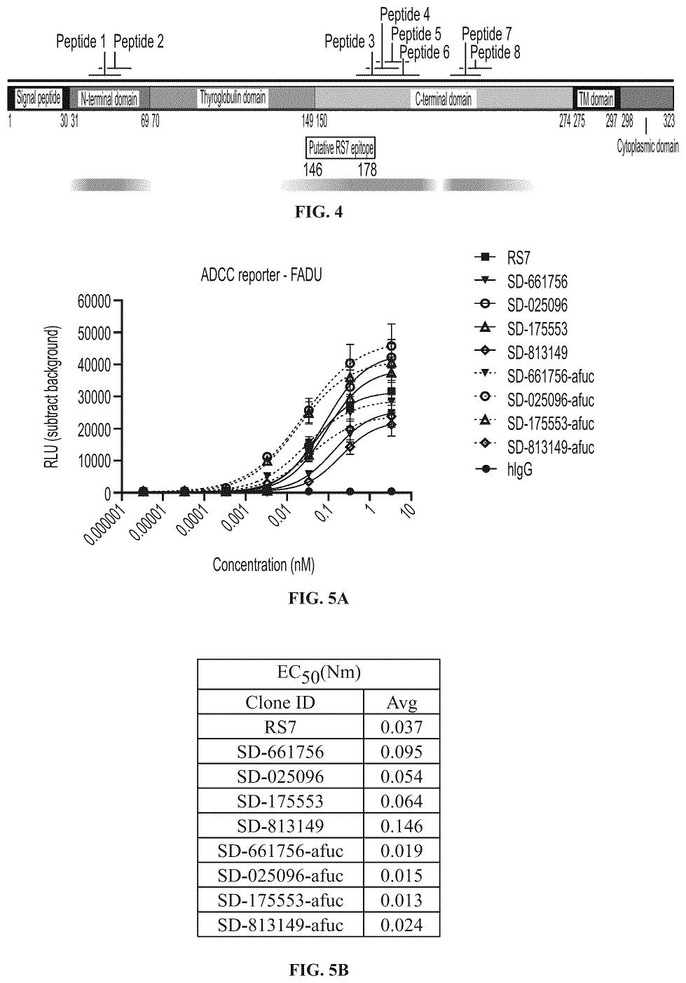

FIG. 4 shows the peptide mapping of the antibodies by bin group. Sequence map showing the location of 8 peptides bound by anti-TROP2 antibodies according to SPR data. Not every antibody tested recognized a linear epitope in this assay. Some antibodies within bin Group 3 recognized Peptides 1-2, suggesting an epitope somewhere near the N-terminus of the TROP2 protein. A subset of antibodies that bin together in Groups 1 and 2 recognized Peptides 3-6 in the C-terminal domain near to the putative RS7 epitope, suggesting that those antibodies in those bins target an area at or near those sites. Lastly, a subset of antibodies in Group 4 recognized Peptides 7-8 and thus the group may broadly target the C-terminal region of the C-terminal domain, just proximal to the transmembrane domain and separate from the RS7 epitope.

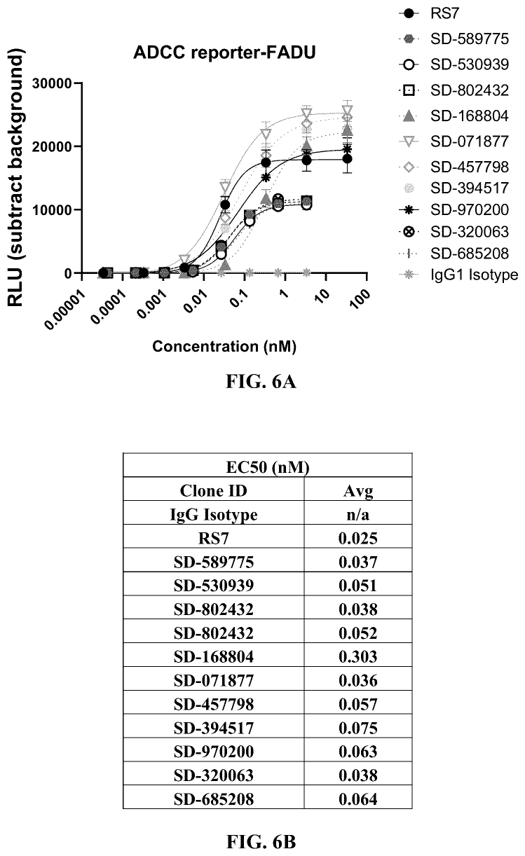

FIGS. 5A and 5B show the results from the ADCC reporter assay for the hits of the mouse antibodies taught herein. Dose-response graph in FIG. 5A shows the ADCC potency of Anti-human Trop2 antibodies in human head and neck cancer cell line FaDu. NFAT CD16 Jurkat ADCC reporter cell lines is used to show ADCC potency by Anti-human Trop2 antibodies and afucosylated anti-human Trop2 antibodies. FIG. 5B shows EC₅₀ values for each tested antibody. All afucosylated anti-human Trop2 antibodies show much smaller EC₅₀ value in compared to their fucosylated version, indicating that afucosylated anti-human Trop2 antibodies enhance their ADCC potency. EC₅₀ values are averages of n=2-3 experiments.

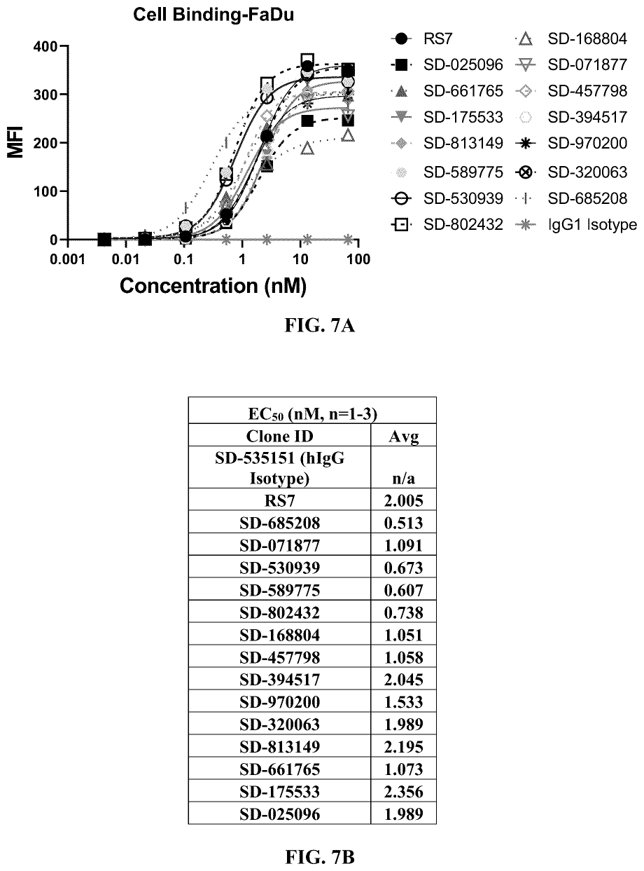

FIGS. 6A and 6B is a graph that shows the results from the ADCC reporter assay for hits of the chicken antibodies taught herein. FIG. 6A is a graph that shows ADCC potency of Anti-human Trop2 antibodies in FaDu cancer cell line. Dose-response graph shows the ADCC potency of Anti-human Trop2 antibodies in FaDu cancer cell line. NFAT CD16 Jurkat ADCC reporter cell lines is used to show ADCC potency by Anti-human Trop2 antibodies. Most of the clone shows very similar ADCC potency in compared to RS7. EC₅₀ values are averages of n=1-3 experiments. ADCC potency of Anti-human Trop2 antibodies in Fadu cancer cell line. FIG. 6B. Table which shows that most of the clone shows very similar ADCC potency in compared to RS7. EC50 values are averages of n=1-3 experiments.

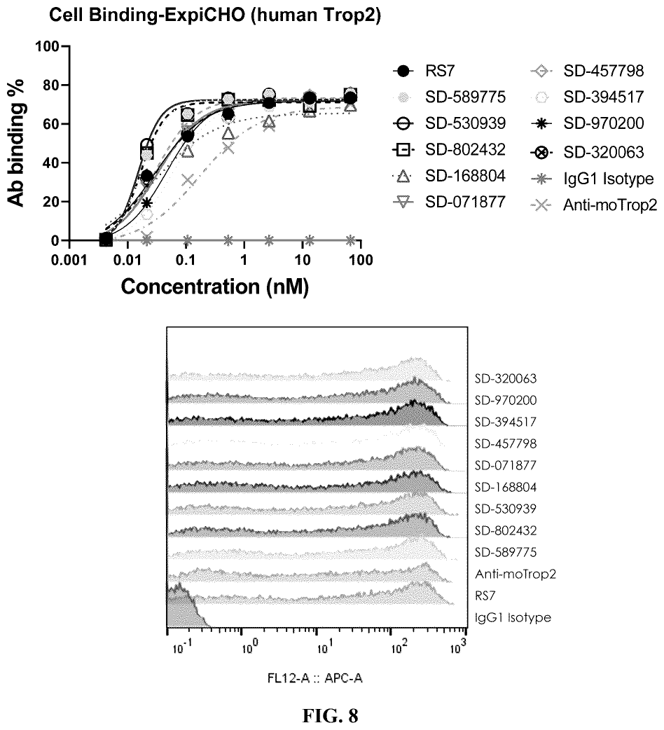

FIGS. 7A and 7B show cell binding results for the antibodies taught herein. Anti-human Trop2 antibodies binding potency to human head and neck cancer cell line FaDu. FACS analysis shows in FIG. 7A anti-Trop2 antibodies and RS7 binding to FaDu cells that endogenously expresses human Trop2. Values plotted are Median Fluorescent Intensity. EC₅₀ values in FIG. 7B are averages of n=1-3 experiments.

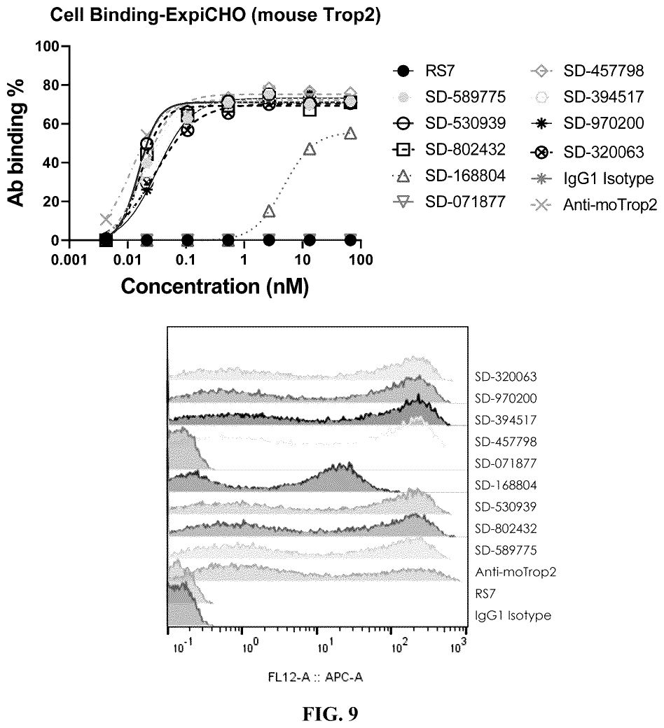

FIG. 8 is a graph that shows the results from anti-human Trop2 antibodies derived from immunized chickens binding to ExpiCHO cells overexpressing human Trop2. FACS analysis showing anti-Trop2 antibodies and RS7 binding to ExpiCHO cells overexpressing mouse Trop2 in a dose-dependent manner. Values plotted are percent of antibody binding to target cells. Top dose (66.67 nM) in binding assay is used for histogram overlay comparing antibody binding levels to human Trop2 for the antibodies tested.

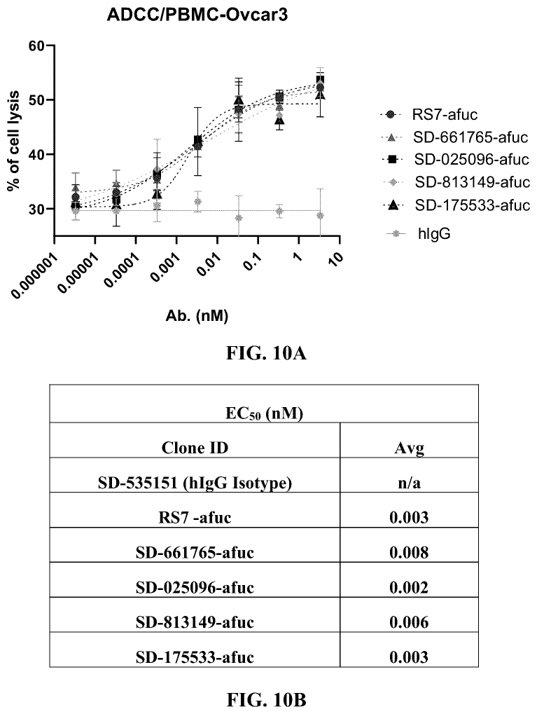

FIG. 9 is a graph that shows the results from anti-human Trop2 antibodies derived from immunized chickens binding to ExpiCHO cells overexpressing mouse Trop2. FACS analysis showing select anti-Trop2 antibodies binding to ExpiCHO cells overexpressing mouse Trop2 in a dose-dependent manner, indicating cross-reactivity with mouse Trop2. RS7 control, which is specific to human Trop2 only, does not show binding to ExpiCHO cells overexpressing mouse Trop2. Values plotted are percent of antibody binding to target cells. Top dose (66.67 nM) in binding assay is used for histogram overlay comparing antibody binding levels to human Trop2 for the antibodies tested.

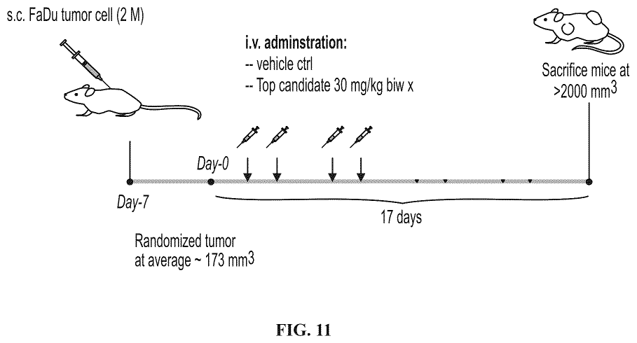

FIGS. 10A and 10B show peripheral blood mononuclear cell (PBMC) killing with the anti-human Trop2 antibodies of the present invention, which exhibit strong human ovarian cancer (Ovcar3) cell killing. The graph in FIG. 10A shows FACS analysis of cell death triggered by anti-TROP2 molecules against TROP2 expressing Ovcar3 target cells and human PBMC as effector cells. FIG. 10B contains EC₅₀ values for each tested antibody.

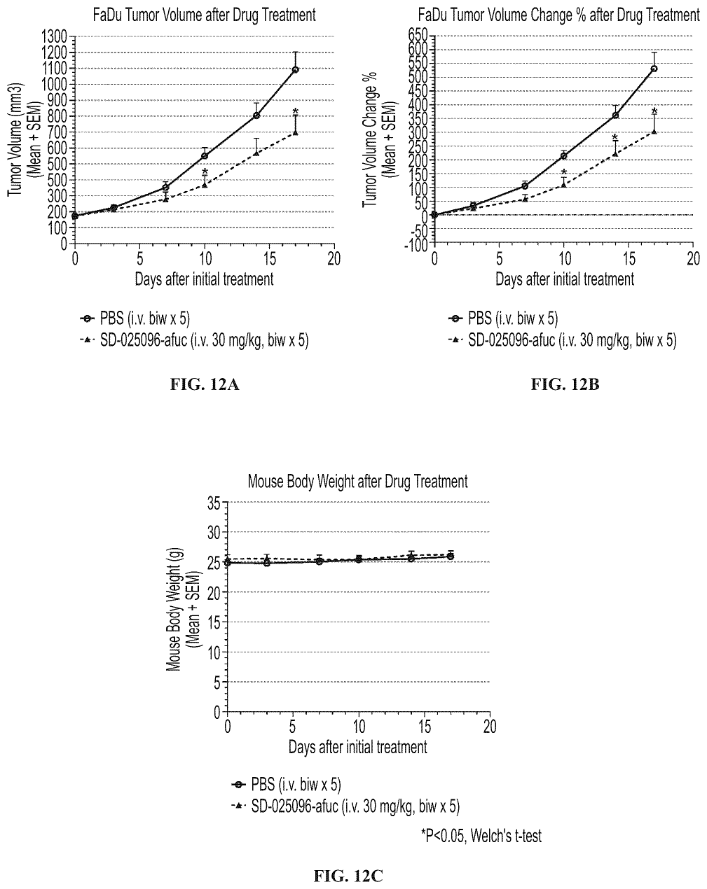

FIG. 11 is an illustration of the study design for in vivo effectiveness.

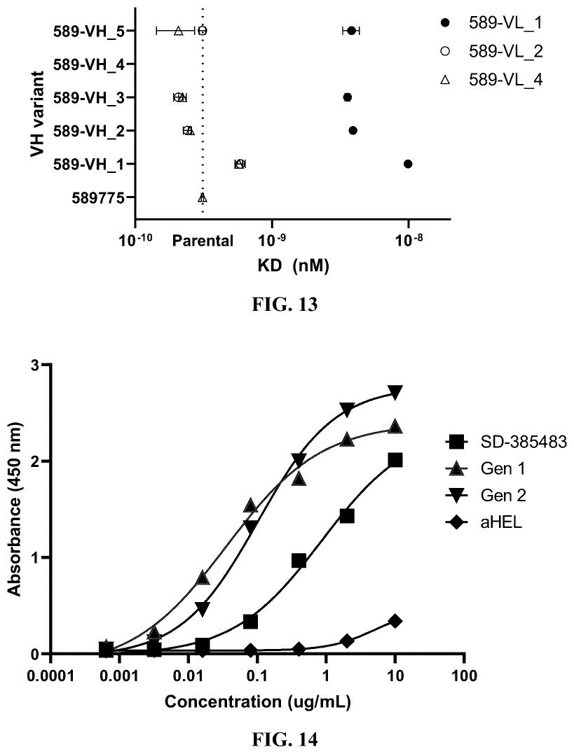

FIGS. 12A to 12C are graphs that show the results from the in vivo study using FaDu xenograft in nude mice. Randomization and grouping was performed on Day 0, based on tumor size. Dosing was performed on Days 0, 3, 7, 10, and 14 (FIG. 12A) Tumor volume for FaDu nude mice xenograft model. (FIG. 12B) Tumor volume percent change. (FIG. 12C) Mouse body weight

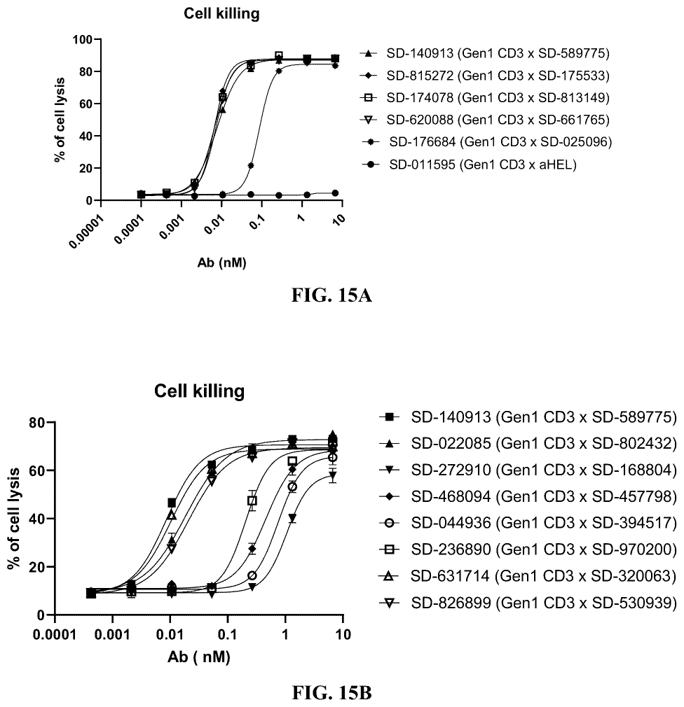

FIG. 13 shows Humanization of SD-589775. Binding kinetics to huTROP2 were measured using human Fc-capture on the Carterra LSA high-throughput SPR instrument (Carterra, Inc.). Combinations of humanized VH and VL sequences were tested and compared to the parental chimera. No variants with 589-VH_4 or 589-VL_3 showed measurable binding to huTROP2. 589-VH_5 and 589-VL_4 refer to the parental sequences.

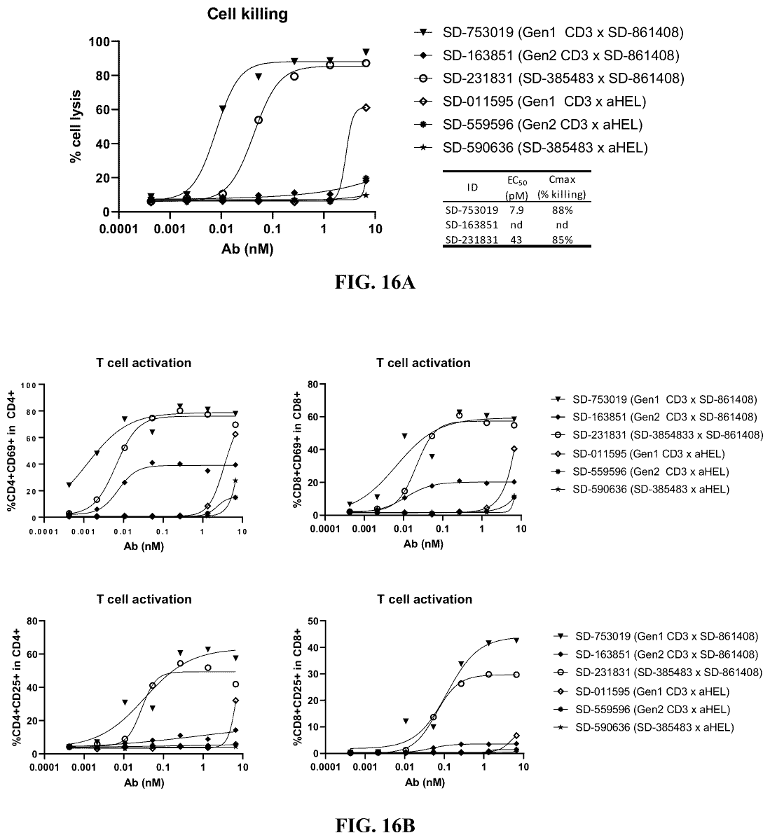

FIG. 14 shows ELISA data showing binding of anti-CD3 antibodies to CD3ϵδ heterodimer. Plate coated with antibody, and then detected by dose response of CD3ϵδ-HRP.

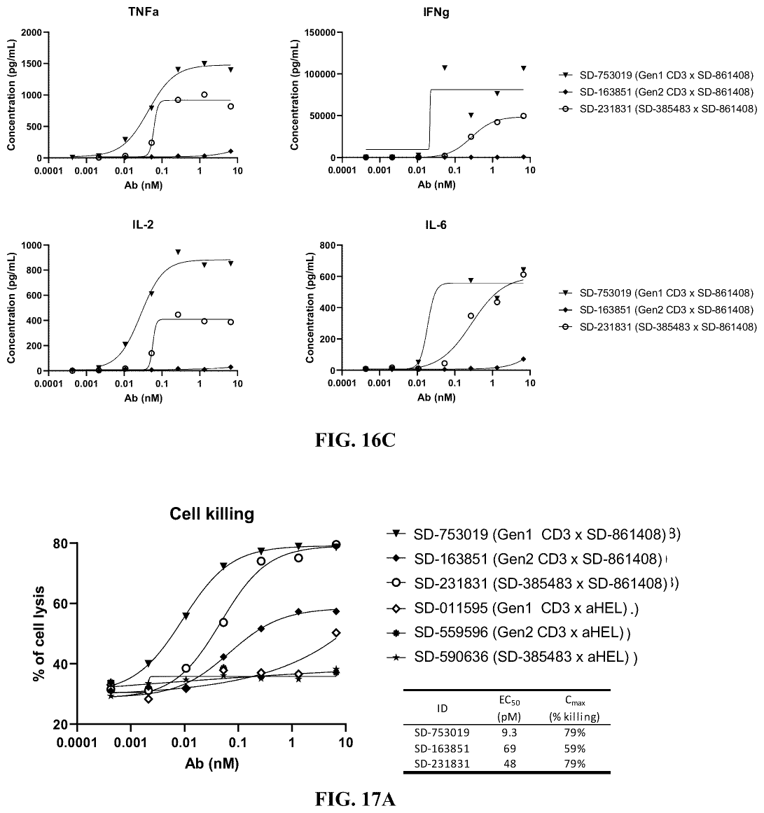

FIG. 15 shows TROP2×CD3 TAA binding arm bispecific screening. Cell killing via coculture of PBMCs and FaDu tumor cells. % cell lysis measured after 48 hours. FIG. 15A shows mouse-derived TROP2 binding antibodies (as well as SD-589775). FIG. 15B shows chicken-derived TROP2 binding antibodies.

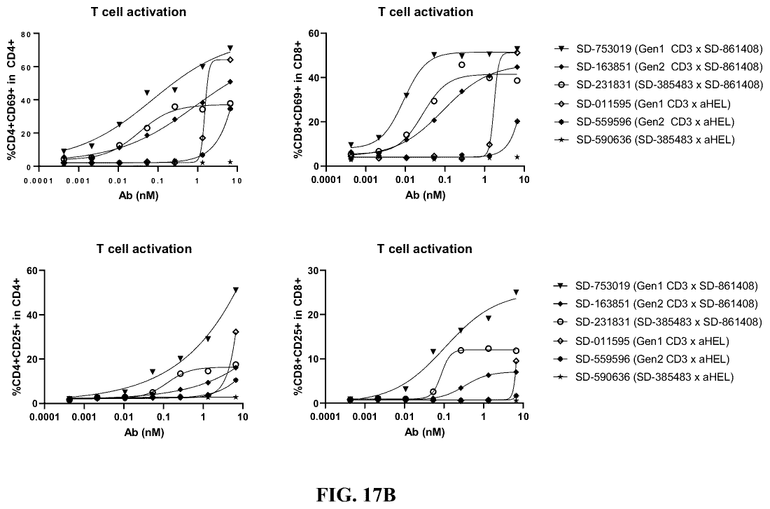

FIGS. 16A to 16C show TROP2×CD3 bispecific cell killing characterization against FaDu. FIG. 16A shows cell killing via coculture of PBMCs and FaDu tumor cells. % cell lysis measured after 48 hours. FIG. 16B shows T cell activation induced by coculture of PBMCs with FaDu tumor cells. T cell activation measured after 48 hours. FIG. 16C shows cytokine production and release induced by coculture of PBMCs with FaDu tumor cells. Cytokine levels measured after 24 hours.

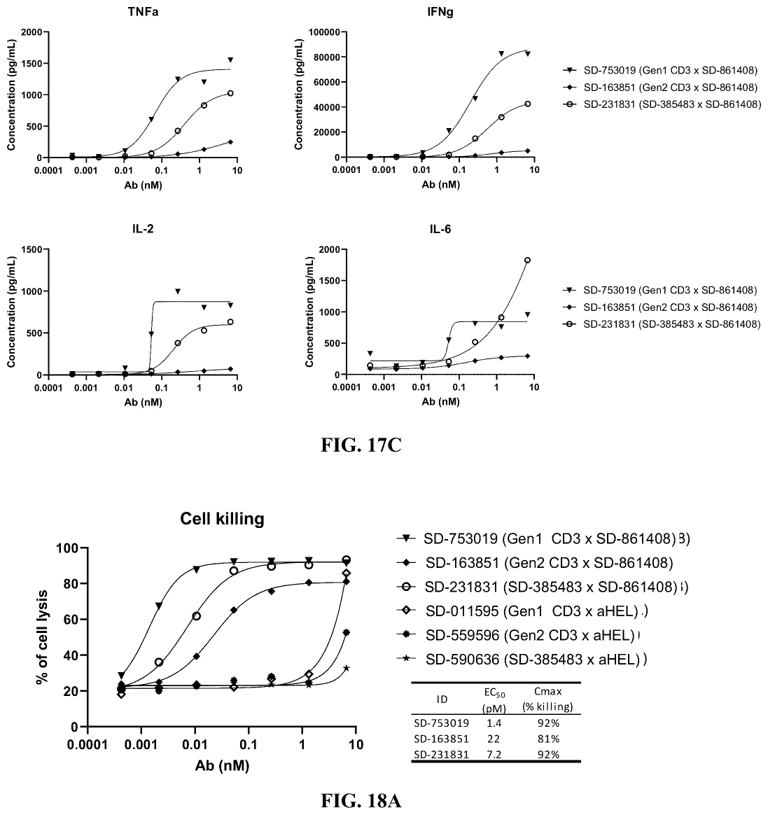

FIGS. 17A to 17C show TROP2×CD3 bispecific cell killing characterization against Calu-3. FIG. 17A shows cell killing via coculture of PBMCs and Calu-3 tumor cells. % cell lysis measured after 48 hours. FIG. 17B shows T cell activation induced by coculture of PBMCs with Calu-3 tumor cells. T cell activation measured after 48 hours. FIG. 17C shows cytokine production and release induced by coculture of PBMCs with Calu-3 tumor cells. Cytokine levels measured after 24 hours.

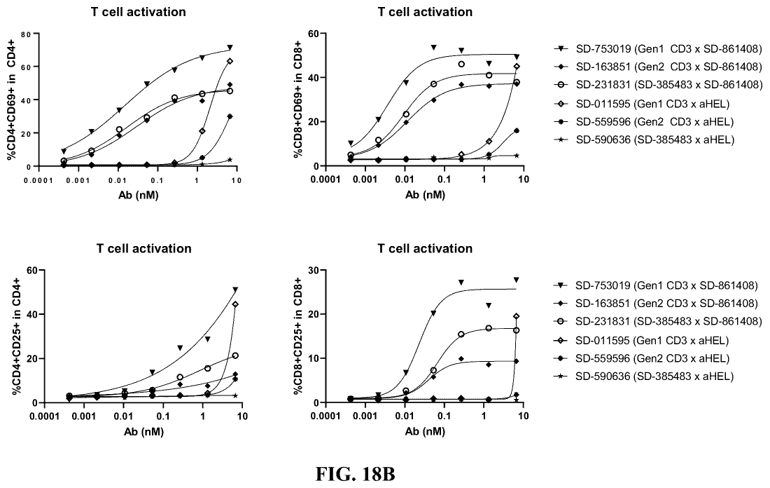

FIGS. 18A to 18C show TROP2×CD3 bispecific cell killing characterization against OvCar-3. FIG. 18A shows cell killing via coculture of PBMCs and OvCar-3 tumor cells. % cell lysis measured after 48 hours. FIG. 18B shows T cell activation induced by coculture of PBMCs with OvCar-3 tumor cells. T cell activation measured after 48 hours. FIG. 18C shows cytokine production and release induced by coculture of PBMCs with OvCar-3 tumor cells. Cytokine levels measured after 24 hours.

In vivo efficacy study design—SD-174078 multiple doses.

Eleven-week-old female NSG with MHC I/II double knockout nude mice (Jackson Laboratory, Cat. #025216) were used in this assay. 20×10\hat{ }6 human PBMC (STEMCELL Technologies; ID CE0006634) were resuspended in 100 μl PBS and engrafted into each mouse through intravenous injection. Mice were grouped randomly with similar averaged body weight on the 13th day after human PBMC engraftment, and each group had 5-8 mice. At this time, 2×10\hat{ }6 FaDu cells in 100 μl of PBS and MatriGel (Corning, Cat. #354234) (v:v=1:1) were inoculated to the left upper side of each mouse by subcutaneous injection. Human bispecific TROP2×CD3 antibody (was made in a stock solution of 0.1 mg/ml in PBS. For each mouse, the volume of administered vehicle control (PBS) or antibody was calculated by the formula Volume (μl)=Mouse body weight (g)×10 μl/g. PBS and SD-174078 were administered on days 0, 3, and 7, for 3 times total (biw×3) via intravenous injection. For each individual tumor, the longest longitudinal diameter as length and the widest transverse diameter as width were measured by using a Traceable Digital Calliper (VWR, Cat. #62379-531). Tumor volume (TV) were then calculated by the formula TV=[length×(width)\hat{ }2]/2.

Human bispecific TROP2×CD3 antibody comprises: SD-174078_knob: CD3 Gen 1 (SEQ ID NO:174), SD-174078_hole: 813149_VL (SEQ ID NO:126), SD-231831 knob: CD3 Gen 2 (190), SD-231831_hole: 589_VL2 (SEQ ID NO:166). The skilled artisan will recognize that the knob and hole chains can be interchanged, or can be changed to other knob and hole variants, or for different Fc variants and/or isotypes.

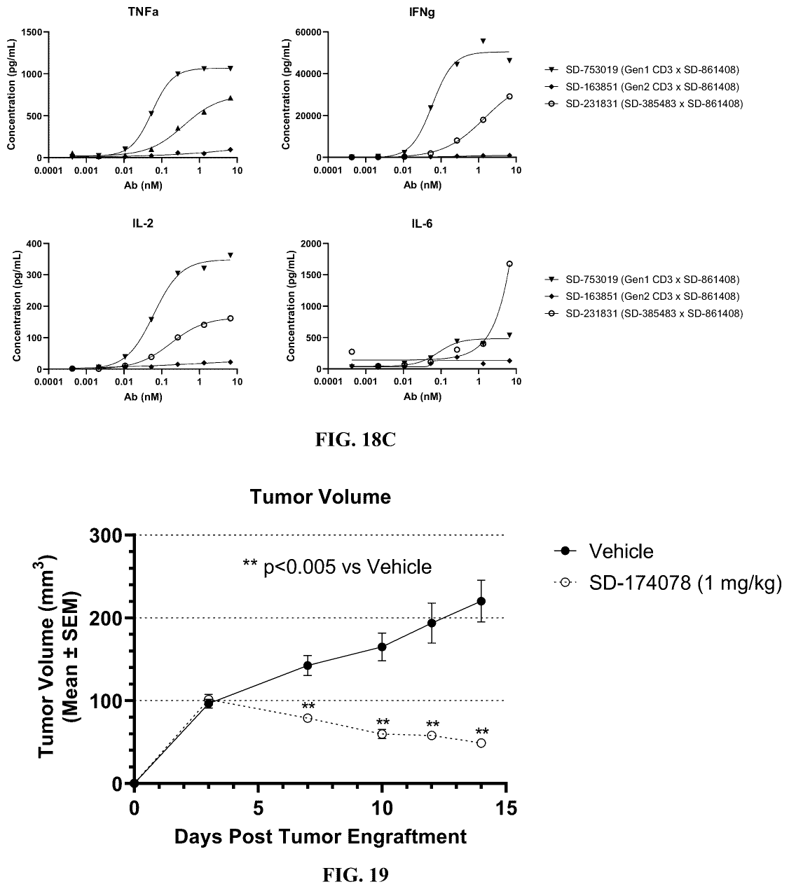

FIG. 19 is a graph that shows the results from the in vivo efficacy study using multiple doses of SD-174078. FaDu xenograft in humanized NSG MHC I/II dKO mice. Mice were engrafted with 20×10\hat{ }6 human PBMCs at day −14 via intravenous injection. At day −1, 2×10\hat{ }6 FaDu cells were engrafted subcutaneously. At days 0, 3, and 7, mice were dosed with either PBS (vehicle) or SD-174078 (1 mg/kg) via intravenous injection. Tumor volume is shown. Significance was determined using Welch's T-test.

In vivo efficacy study design—SD-231831 single dose.

Eleven-week-old female NSG with MHC I/II double knockout nude mice (Jackson Laboratory, Cat. #025216) were used in this assay. 20×10\hat{ }6 human PBMC (STEMCELL Technologies; ID CE0006634) were resuspended in 100 μl PBS and engrafted into each mouse through intravenous injection. Mice were grouped randomly with similar averaged body weight on the 13th day after human PBMC engraftment, and each group had 5-8 mice. At this time, 2×10\hat{ }6 FaDu cells in 100 μl of PBS and MatriGel (Corning, Cat. #354234) (v:v=1:1) were inoculated to the left upper side of each mouse by subcutaneous injection. Human bispecific antibody (TROP2×CD3) was made in a stock solution of 0.1 mg/ml in PBS. For each mouse, the volume of administered vehicle control (PBS) or antibody was calculated by the formula Volume (μl)=Mouse body weight (g)×10 μl/g. PBS and SD-231831 were administered on day 7, a single dose via intravenous injection. For each individual tumor, the longest longitudinal diameter as length and the widest transverse diameter as width were measured by using a Traceable Digital Calliper (VWR, Cat. #62379-531). Tumor volume (TV) were then calculated by the formula TV=[length×(width)\hat{ }2]/2.

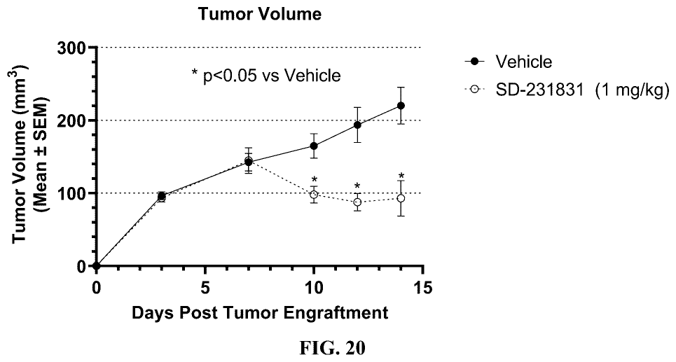

FIG. 20 is a graph that shows the results using a single dose. FaDu xenograft in humanized NSG MHC I/II dKO mice. Mice were engrafted with 20×10\hat{ }6 human PBMCs at day −14 via intravenous injection. At day −1, 2×10\hat{ }6 FaDu cells were engrafted subcutaneously. At day 7, mice were given a single dose of either PBS (vehicle) or SD-231831 (1 mg/kg) via intravenous injection. Tumor volume is shown. Significance was determined using Welch's T-test.

The skilled artisan will recognize that antibodies that exhibit little or no binding to a target antigen can be described as having a low affinity, and a high equilibrium dissociation constant (KD) for the target antigen. The skilled artisan will also recognize that antibodies that exhibit little or no binding to a collective assembly of target antigenic epitopes can be described as having a low avidity, and a high equilibrium dissociation constant (KD) for the collective assembly of target antigenic epitopes.

In some embodiments, provided herein are anti-Trop 2 antibodies having a binding affinity (KD) to Trop 2 of about 5 μM to about 5 pM, about 1 μM to about 5 pM, about 0.5 μM to about 5 pM, about 0.1 μM to about 5 pM, about 50 nM to about 5 pM, about 10 nM to about 5 pM, about 5nM to about 5pM, about InM to about 5pM, about 0.5 nM to about 5 pM, about 0.1 nM to about 5 pM, about 50 pM to about 5 pM, about 10 pM to about 5 pM.

In some embodiments, anti-Trop 2 antibodies have a binding avidity (KD) to Trop 2 of about 500 nM to about 0.1 pM, about 100 nM to about 0.1 pM, about 50 nM to about 0.1 pM, about 10 nM to about 0.1 pM, about 5 nM to about 0.1 pM, about 1 nM to about 0.1 pM, about 0.5 nM to about 0.1 pM, about 0.1 nM to about 0.1 pM, about 50 pM to about 0.1 pM, about 10 pM to about 0.1 pM, about 5 pM to about 0.1 pM, about 1 pM to about 0.1 pM, about 0.5 pM to about 0.1 pM.

In some embodiments, anti-Trop 2 antibodies have a half maximal effective concentration (EC₅₀) to Trop 2 of about 500 nM to about 0.001 nM, about 100 nM to about 0.001 nM, about 50 nM to about 0.001 nM, about 10 nM to about 0.001 nM, about 5 nM to about 0.001 nM, about 1 nM to about 0.001 nM, about 0.5 nM to about 0.001 nM, about 0.1 nM to about 0.001 nM, about 0.05 nM to about 0.001 nM, about 0.01 nM to about 0.001 nM, about 0.005 nM to about 0.001 nM.

The skilled artisan will recognize that binding specificity may be determined through a series of competition binding paradigms, in which a desired antibody demonstrates its ability to prevent binding of a known reference antibody to its target epitope at varying concentrations. In some embodiments, the reference antibody is RS7. In some embodiments, the reference RS7 antibody, binds the epitope at amino acids 146 to 178. The skilled artisan will also recognize that the reference RS7 antibody may be utilized as control antibodies in agonist and antagonist assays.

In some embodiments, the anti-Trop 2 antibody is a full-length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of the anti-Trop 2 antibody is from a human IgG1, human IgG2, human IgG3, or human IgG4.

Exemplary anti-Trop 2 Antibodies-CDR Sequences.

| | | SEQ |
|---|---|---|
| Sequence | | ID |
| description | Amino Acid Sequence | NO: |
| SD- | QVQLQQSGAELVRPGASVKLSCKASGYTFTSYWINWVKQRPGQGLE | 1 |
| 025096_VH | WIGNIFPSGSYTNYNQKFKDRATLTVDKSSSTAFMQLSSPTSEDSAIYF | |
| | CTRGSGFDYWGQGTTLTVSS | |
| SD- | SYWIN | 2 |
| 025096_VH_ | | |
| CDR1 | | |
| SD- | NIFPSGSYTNYNQKFKD | 3 |
| 025096_VH_ | | |
| CDR2 | | |
| SD- | GSGFDY | 4 |
| 025096_VH_ | | |
| CDR3 | | |
| SD- | CAGGTTCAGCTGCAGCAGTCTGGGGCTGAGCTGGTGAGGCCTGG | 5 |
| 025096_VH_ | GGCTTCAGTGAAGCTGTCCTGCAAGGCTTCTGGCTACACCTTCACC | |
| DNA | AGCTACTGGATAAACTGGGTGAAGCAGAGGCCTGGACAAGGCCTT | |
| | GAGTGGATCGGAAATATTTTTCCTTCTGGTAGTTATACTAACTACA | |
| | ATCAAAAGTTCAAGGACAGGGCCACATTGACTGTAGACAAATCCT | |
| | CCAGCACAGCCTTCATGCAACTCAGCAGCCCGACATCTGAGGACTC | |
| | TGCGATCTATTTCTGTACAAGGGGTAGTGGCTTCGACTACTGGGGC | |
| | CAAGGCACCACTCTCACGGTCTCCTCA | |
| SD- | DIVLTQSPATLSVTPGDSVSLSCRASQSISNNLHWYQQKSHESPRLLIK | 6 |
| 025096_VL | YASQSISGIPSRFSGSGSGTDFTLSINSVETEDFGMYFCQQSNSWPFTF | |
| | GSGTKLEIK | |
| SD- | RASQSISNNLH | 7 |
| 025096_VL_ | | |
| CDR1 | | |
| SD- | YASQSIS | 8 |
| 025096_VL_ | | |
| CDR2 | | |
| SD- | QQSNSWPFT | 9 |
| 025096_VL_ | | |
| CDR3 | | |
| SD- | GATATTGTGCTAACCCAGTCTCCAGCCACCCTGTCTGTGACTCCAG | 10 |
| 025096_VL_ | GAGATAGCGTCAGTCTTTCCTGCAGGGCCAGCCAAAGTATTAGCA | |
| DNA | ACAACCTACACTGGTATCAACAAAAATCACATGAGTCTCCAAGGCT | |
| | TCTCATCAAGTATGCTTCCCAGTCCATCTCTGGGATCCCCTCCAGGT | |
| | TCAGTGGCAGTGGATCAGGGACAGATTTCACTCTCAGTATCAACA | |
| | GTGTGGAGACTGAAGATTTTGGAATGTATTTCTGTCAACAGAGTAA | |
| | CAGCTGGCCATTCACGTTCGGCTCGGGGACAAAGTTGGAAATAAA | |
| | A | |
| SD- | AVTLDESGGGLQTPGGALSLVCKASGFDFSGYGMGWVRQAPGKGLE | 11 |
| 071877_VH | WVAGITNDGRYIGHGSAVKGRATISRDNGQSTVRLQLNNLRAEDTG | |
| | TYFCTKCAYRSGCDYEAGSIDAWGHGTEVIVSS | |
| SD- | GYGMG | 12 |
| 071877_VH_ | | |
| CDR1 | | |
| SD- | GITNDGRYIGHGSAVKG | 13 |
| 071877_VH_ | | |
| CDR2 | | |
| SD- | CAYRSGCDYEAGSIDA | 14 |
| 071877_VH_ | | |
| CDR3 | | |
| SD- | GCCGTGACGTTGGACGAGTCCGGGGGGGGCCTCCAGACGCCCGG | 15 |
| 071877_VH_ | AGGAGCGCTCAGCCTCGTCTGCAAGGCCTCCGGGTTTGACTTCAG | |
| DNA | CGGTTATGGCATGGGTTGGGTGCGCCAGGCGCCCGGCAAAGGGC | |
| | TGGAATGGGTCGCTGGTATTACCAATGATGGTAGATATATAGGCC | |
| | ACGGGTCGGCGGTGAAGGGCCGTGCCACCATCTCGAGGGACAAC | |
| | GGGCAGAGCACAGTGAGGCTGCAGCTGAACAACCTCAGGGCTGA | |
| | GGACACCGGCACCTACTTCTGCACGAAATGTGCTTATCGTAGTGGT | |
| | TGTGATTATGAAGCTGGTTCTATCGACGCATGGGGCCACGGGACC | |
| | GAAGTCATCGTCTCCTCC | |
| SD- | ALALTQPSSVSANLGGTVKITCSGDNNWYGWYQQKSPGSAPVTVIYY | 16 |
| 071877_VL | DDKRPSDIPSRFSGSRSGSTGTLTITGVQAEDEAVYFCGNRDSSAGYV | |
| | GIFGAGTTLTVL | |
| SD- | SGDNNWYG | 17 |
| 071877_VL_ | | |
| CDR1 | | |
| SD- | YDDKRPS | 18 |
| 071877_VL_ | | |
| CDR2 | | |
| SD- | GNRDSSAGYVGI | 19 |
| 071877_VL_ | | |
| CDR3 | | |
| SD- | GCACTTGCGCTGACTCAGCCGTCCTCGGTGTCAGCAAACCTGGGA | 20 |
| 071877_VL_ | GGAACCGTCAAGATCACCTGCTCCGGGGATAACAATTGGTATGGC | |
| DNA | TGGTATCAGCAGAAGTCACCTGGCAGTGCCCCTGTCACTGTGATCT | |
| | ATTACGACGACAAGAGACCCTCGGACATCCCTTCACGATTCTCCGG | |
| | TTCCAGATCCGGCTCCACGGGCACATTAACCATCACTGGGGTCCAA | |
| | GCCGAGGACGAGGCTGTCTATTTCTGTGGAAACAGGGACAGCAGT | |
| | GCTGGTTATGTTGGTATATTTGGGGCCGGGACAACCCTGACCGTCC | |
| | TA | |
| SD- | AVTLDESGGGLQTPGGALSLVCKASGFTFSDRGMGWVRQVPGKGLE | 21 |
| 168804_VH | WVAAISSTVTYTSYGPAVKGRATISRDDGQSTVRLQLNNLRAEDTGTY | |
| | YCAKGPYRGIYGSEIDAWGHGTEVIVSS | |
| SD- | DRGMG | 22 |
| 168804_VH_ | | |
| CDR1 | | |
| SD- | AISSTVTYTSYGPAVKG | 23 |
| 168804_VH_ | | |
| CDR2 | | |
| SD- | GPYRGIYGSEIDA | 24 |
| 168804_VH_ | | |
| CDR3 | | |
| SD- | GCCGTGACGTTGGACGAGTCCGGGGGGGGCCTCCAGACGCCCGG | 25 |
| 168804_VH_ | AGGAGCGCTCAGCCTCGTCTGCAAGGCCTCCGGGTTCACCTTCAGT | |
| DNA | GACCGTGGCATGGGTTGGGTGCGACAGGTGCCCGGCAAGGGGCT | |
| | GGAGTGGGTCGCTGCTATTAGCAGCACTGTCACTTACACATCATAT | |
| | GGGCCGGCGGTGAAGGGCCGTGCCACCATCTCGAGGGACGATGG | |
| | GCAGAGCACAGTGAGGCTGCAACTGAACAACCTCAGGGCTGAGG | |
| | ACACCGGCACCTACTACTGCGCCAAGGGTCCTTACCGTGGTATTTA | |
| | TGGTAGTGAAATCGACGCATGGGGCCACGGGACCGAAGTCATCGT | |
| | CTCCTCC | |
| SD- | ALALTQPSSVSANPGETVKITCSGGSYSYGSYYYGWYQQKSPGSAPVT | 26 |
| 168804_VL | VIYDNDKRPSGIPSRFSGSKSGSTGTLTITGVQAEDEAVYFCGSTDSSST | |
| | AAFGAGTTLTVL | |
| SD- | SGGSYSYGSYYYG | 27 |
| 168804_VL_ | | |
| CDR1 | | |
| SD- | DNDKRPS | 28 |
| 168804_VL_ | | |
| CDR2 | | |
| SD- | GSTDSSSTAA | 29 |
| 168804_VL_ | | |
| CDR3 | | |
| SD- | GCACTTGCGCTGACTCAGCCGTCCTCGGTGTCAGCAAACCCGGGA | 30 |
| 168804_VL_ | GAAACCGTCAAGATCACCTGCTCTGGGGGCAGCTATAGCTATGGA | |
| DNA | AGTTACTATTATGGCTGGTACCAGCAGAAGTCACCTGGCAGTGCCC | |
| | CAGTCACTGTGATCTATGACAACGACAAGAGACCCTCGGGCATCCC | |
| | TTCACGATTCTCCGGTTCCAAATCCGGCTCCACGGGCACATTAACC | |
| | ATCACTGGGGTCCAAGCCGAGGACGAGGCTGTCTATTTCTGTGGG | |
| | AGTACAGACAGCAGCAGTACTGCTGCATTTGGGGCCGGGACAACC | |
| | CTGACCGTCCTA | |
| SD- | QIQLQESGPGLMKPSQSLSLTCTVTGYSITSDYAWNWIRQFPGNKLE | 31 |
| 175533_VH | WMGHITYSGSTSYNPSLKSRISISRDTSKNQFFLQLNSVTTGDTATYYC | |
| | ARGGSTLISGDFDYWGQGTTLTVSS | |
| SD- | SDYAWN | 32 |
| 175533_VH_ | | |
| CDR1 | | |
| SD- | HITYSGSTSYNPSLKS | 33 |
| 175533_VH_ | | |
| CDR2 | | |
| SD- | GGSTLISGDFDY | 34 |
| 175533_VH_ | | |
| CDR3 | | |
| SD- | CAGATTCAGCTTCAGGAGTCAGGACCTGGCCTAATGAAACCTTCTC | 35 |
| 175533_VH_ | AGTCTCTGTCCCTCACCTGCACTGTCACTGGCTACTCAATCACCAGT | |
| DNA | GATTATGCCTGGAACTGGATCCGGCAGTTTCCAGGAAACAAACTG | |
| | GAGTGGATGGGCCACATAACCTACAGTGGTAGCACTAGCTACAAC | |
| | CCATCTCTCAAAAGTCGAATCTCTATCTCTCGAGACACATCCAAGA | |
| | ACCAGTTCTTCCTGCAGTTGAATTCTGTGACTACTGGGGACACAGC | |
| | CACATATTACTGTGCAAGAGGGGGATCTACTTTGATTTCAGGGGAC | |
| | TTTGACTACTGGGGCCAAGGCACCACTCTCACAGTCTCCTCA | |
| SD- | DIVLTQSHKFMSTSVGDRVSITCKASQDVSTAVAWYQQKPGQSPKLLI | 36 |
| 175533_VL | YSASYRYTGVPDRFTGSGSGTDFTFTISSVQAEDLAVYYCQQHYSTPLT | |
| | FGAGTKLELK | |
| SD- | KASQDVSTAVA | 37 |
| 175533_VL_ | | |
| CDR1 | | |
| SD- | SASYRYT | 38 |
| 175533_VL_ | | |
| CDR2 | | |
| SD- | QQHYSTPLT | 39 |
| 175533_VL_ | | |
| CDR3 | | |
| SD- | GACATTGTGCTGACCCAATCTCACAAATTCATGTCCACATCAGTAG | 40 |
| 175533_VL_ | GAGACAGGGTCAGCATCACCTGCAAGGCCAGTCAGGATGTGAGTA | |
| DNA | CTGCTGTAGCCTGGTATCAACAGAAACCAGGACAATCTCCTAAACT | |
| | ACTGATTTACTCGGCATCCTACCGGTACACTGGAGTCCCTGATCGC | |
| | TTCACTGGCAGTGGATCTGGGACGGATTTCACTTTCACCATCAGCA | |
| | GTGTGCAGGCTGAAGACCTGGCAGTTTATTACTGTCAGCAACATTA | |
| | TAGTACTCCTCTCACGTTCGGTGCTGGGACCAAGCTGGAGCTGAA | |
| | A | |
| SD- | AVTLDESGGGLQTPGGGLSLVCKASGFDFIDYDMFWVRQAPGKGLA | 41 |
| 320063_VH | WVGVINRSGSYTNYAPAVKGRATISRDNGQSTVRLQLNNLRAEDTGT | |
| | YYCAKGVCTHCWDADSAGNIDAWGHGTEVIVSS | |
| SD- | DYDMF | 42 |
| 320063_VH_ | | |
| CDR1 | | |
| SD- | VINRSGSYTNYAPAVKG | 43 |
| 320063_VH_ | | |
| CDR2 | | |
| SD- | GVCTHCWDADSAGNIDA | 44 |
| 320063_VH_ | | |
| CDR3 | | |
| SD- | GCCGTGACGTTGGACGAGTCCGGGGGCGGCCTCCAGACGCCTGG | 45 |
| 320063_VH_ | AGGAGGGCTCAGCCTCGTCTGCAAGGCCTCCGGGTTCGACTTCAT | |
| DNA | CGATTATGACATGTTCTGGGTGCGACAGGCTCCAGGCAAGGGGCT | |
| | GGCATGGGTCGGTGTTATTAACAGGAGTGGTAGTTACACAAACTA | |
| | CGCGCCGGCGGTGAAGGGCCGTGCCACCATCTCGAGGGACAACG | |
| | GGCAGAGCACAGTGAGGCTGCAGCTGAACAACCTCAGGGCTGAG | |
| | GACACCGGCACCTACTACTGCGCCAAAGGTGTATGTACTCATTGTT | |
| | GGGATGCAGACAGTGCCGGTAACATCGACGCATGGGGCCACGGG | |
| | ACCGAAGTCATCGTCTCCTCC | |
| SD- | ALALTQPSSVSANPGETVEITCSGGSGNYGWFQQKSPGSAPVTVIYN | 46 |
| 320063_VL | NNYRLSDIPSRFSGSASGSTATLTITGVRVEDEAVYFCGSADSTYVGLF | |
| | GAGTTLTVL | |
| SD- | SGGSGNYG | 47 |
| 320063_VL_ | | |
| CDR1 | | |
| SD- | NNNYRLS | 48 |
| 320063_VL_ | | |
| CDR2 | | |
| SD- | GSADSTYVGL | 49 |
| 320063_VL_ | | |
| CDR3 | | |
| SD- | GCACTTGCGCTGACTCAGCCGTCCTCGGTGTCAGCGAACCCGGGA | 50 |
| 320063_VL_ | GAAACCGTCGAGATCACCTGCTCCGGGGGTAGTGGCAACTATGGC | |
| DNA | TGGTTCCAGCAGAAGTCTCCTGGCAGTGCCCCTGTCACTGTGATCT | |
| | ATAACAACAACTACAGACTCTCGGACATCCCTTCACGATTCTCCGG | |
| | TTCCGCATCTGGCTCCACAGCCACATTAACCATCACTGGGGTCCGA | |
| | GTCGAGGACGAGGCTGTCTATTTCTGTGGGAGTGCAGACAGCACT | |
| | TATGTTGGTTTATTTGGGGCCGGGACAACCCTGACCGTCCTA | |
| SD- | AVTLDESGGGLQTPGGALSLVCKGSGFTFSSYAMGWVRQAPGKGLE | 51 |
| 394517_VH | YVAAISSGSSTGYGAAVKGRATISRDTGQSTVRLQLNNLRAEDTATYY | |
| | CAKSGYGGSASYVSDIDAWGHGTEVIVSS | |
| SD- | SYAMG | 52 |
| 394517_VH_ | | |
| CDR1 | | |
| SD- | AISSGSSTGYGAAVKG | 53 |
| 394517_VH_ | | |
| CDR2 | | |
| SD- | SGYGGSASYVSDIDA | 54 |
| 394517_VH_ | | |
| CDR3 | | |
| SD- | GCCGTGACGTTGGACGAGTCCGGGGGCGGCCTCCAGACGCCCGG | 55 |
| 394517_VH_ | AGGAGCGCTCAGCCTCGTCTGCAAGGGCTCCGGGTTCACCTTCAG | |
| DNA | CAGTTATGCCATGGGTTGGGTGCGCCAGGCGCCCGGCAAGGGGTT | |
| | GGAATATGTCGCAGCTATTAGCAGTGGTAGTAGCACAGGATACGG | |
| | GGCAGCGGTGAAGGGCCGTGCCACCATCTCGAGGGACACCGGGC | |
| | AGAGCACAGTGAGGCTGCAGCTGAACAACCTCAGGGCTGAGGAC | |
| | ACCGCCACCTACTACTGCGCCAAAAGTGGTTATGGTGGTAGTGCG | |
| | AGCTATGTTAGCGATATCGACGCATGGGGCCACGGGACCGAAGTC | |
| | ATCGTCTCCTCC | |
| SD- | ALALTQPSSVSANPGETVEITCSGGGDYYGTYYYGWYQQKAPGSAPV | 56 |
| 394517_VL | TVIYDNTKRPSNIPSRFSGSTSGSTNTLTITGVQADDEAVYYCGSYDSS | |
| | AGIFGAGTTLTVL | |
| SD- | SGGGDYYGTYYYG | 57 |
| 394517_VL_ | | |
| CDR1 | | |
| SD- | DNTKRPS | 58 |
| 394517_VL_ | | |
| CDR2 | | |
| SD- | GSYDSSAGI | 59 |
| 394517_VL_ | | |
| CDR3 | | |
| SD- | GCACTTGCGCTGACTCAGCCGTCCTCGGTGTCAGCGAACCCGGGA | 60 |
| 394517_VL_ | GAAACCGTTGAGATCACCTGCTCCGGGGGTGGCGACTACTATGGA | |
| DNA | ACTTACTATTATGGCTGGTACCAGCAGAAGGCACCTGGCAGTGCCC | |
| | CTGTCACTGTGATCTATGACAACACCAAGAGACCCTCGAACATCCC | |
| | TTCACGATTCTCCGGTTCCACATCCGGCTCCACAAACACATTAACCA | |
| | TCACTGGGGTCCAAGCCGACGACGAGGCTGTCTATTACTGTGGGA | |
| | GCTACGACAGCAGTGCTGGTATATTTGGGGCCGGGACAACCCTGA | |
| | CCGTCCTA | |
| SD- | AVTLDESGGGLQTPGGALSLVCKGSGFTFSSYAMGWVRQAPGKGLE | 61 |
| 457798_VH | YVAAISSGSSTGYGAAVKGRATISKDTGQSTVRLQLNNLRAEDTATYY | |
| | CAKSGYGGSASYVSDIDAWGHGTEVIVSS | |
| SD- | SYAMG | 62 |
| 457798_VH_ | | |
| CDR1 | | |
| SD- | AISSGSSTGYGAAVKG | 63 |
| 457798_VH_ | | |
| CDR2 | | |
| SD- | SGYGGSASYVSDIDA | 64 |
| 457798_VH_ | | |
| CDR3 | | |
| SD- | GCCGTGACGTTGGACGAGTCCGGGGGCGGCCTCCAGACGCCCGG | 65 |
| 457798_VH_ | AGGAGCGCTCAGCCTCGTCTGCAAGGGCTCCGGGTTCACCTTCAG | |
| DNA | CAGTTATGCCATGGGTTGGGTGCGCCAGGCGCCCGGCAAGGGGTT | |
| | GGAATATGTCGCAGCTATTAGCAGTGGTAGTAGCACAGGATACGG | |
| | GGCAGCGGTGAAGGGCCGTGCCACCATCTCGAAGGACACCGGGC | |
| | AGAGCACAGTGAGGCTGCAGCTGAACAACCTCAGGGCTGAGGAC | |
| | ACCGCCACCTACTACTGCGCCAAAAGTGGTTATGGTGGTAGTGCG | |
| | AGCTATGTTAGCGATATCGACGCATGGGGCCACGGGACCGAAGTC | |
| | ATCGTCTCCTCC | |
| SD- | ALALTQPSSVSANPGETVEITCSGGGDYYGTYYYGWYQQKAPGSAPV | 66 |
| 457798_VL | TVIYDNTKRPSNIPSRFSGSTSGSTNTLTITGVQADDEAIYFCGSYKSNT | |
| | FGAGTTLTVL | |
| SD- | SGGGDYYGTYYYG | 67 |
| 457798_VL_ | | |
| CDR1 | | |
| SD- | DNTKRPS | 68 |
| 457798_VL_ | | |
| CDR2 | | |
| SD- | GSYKSNT | 69 |
| 457798_VL_ | | |
| CDR3 | | |
| SD- | GCACTTGCGCTGACTCAGCCGTCCTCGGTGTCAGCGAACCCGGGA | 70 |
| 457798_VL_ | GAAACCGTTGAGATCACCTGCTCCGGGGGTGGCGACTACTATGGA | |
| DNA | ACTTACTATTATGGCTGGTACCAGCAGAAGGCACCTGGCAGTGCCC | |
| | CTGTCACTGTGATCTATGACAACACCAAGAGACCCTCGAACATCCC | |
| | TTCACGATTCTCCGGTTCCACATCCGGCTCCACAAACACATTAACCA | |
| | TCACTGGGGTCCAAGCCGACGACGAGGCTATCTATTTCTGTGGGA | |
| | GCTACAAAAGTAATACATTTGGGGCCGGGACAACCCTGACCGTCC | |
| | TA | |
| SD- | AVTLDESGGGLQTPGGGLSLVCKASGFDFIDYDMFWVRQAPGKGLE | 71 |
| 530939_VH | WVGVINRSGSYTNYAPAVKGRATISRDNGQSTLRLQLNNLRAEDTGT | |
| | YYCARGACTHCWDADSAGYIDAWGHGTEVIVSS | |
| SD- | DYDMF | 72 |
| 530939_VH_ | | |
| CDR1 | | |
| SD- | VINRSGSYTNYAPAVKG | 73 |
| 530939_VH_ | | |
| CDR2 | | |
| SD- | GACTHCWDADSAGYIDA | 74 |
| 530939_VH_ | | |
| CDR3 | | |
| SD- | GCCGTGACGTTGGACGAGTCCGGGGGGGGCCTCCAGACGCCTGG | 75 |
| 530939_VH_ | AGGAGGGCTCAGCCTCGTCTGCAAGGCCTCCGGGTTCGACTTCAT | |
| DNA | CGATTATGACATGTTCTGGGTGCGACAGGCTCCAGGCAAGGGGCT | |
| | GGAGTGGGTCGGTGTTATTAACAGGAGTGGTAGTTACACAAACTA | |
| | CGCGCCGGCGGTGAAGGGTCGTGCCACCATCTCGAGGGACAACG | |
| | GGCAGAGCACACTGAGGCTGCAGCTGAACAACCTCAGGGCTGAG | |
| | GACACCGGCACCTACTACTGCGCCAGAGGTGCGTGTACTCATTGTT | |
| | GGGATGCAGACAGTGCTGGTTACATCGACGCATGGGGCCACGGG | |
| | ACCGAAGTCATCGTCTCCTCC | |
| SD- | ALALTQPSSVSANPGETVKITCSGGYSGYGWYQQKSPGSAPVTVIYW | 76 |
| 530939_VL | NDKRPSNIPSRFSGSLSGSTNTLIITGVQAEDEAVYFCGGYDSSPGYVG | |
| | IFGAGTTLTVL | |
| SD- | SGGYSGYG | 77 |
| 530939_VL_ | | |
| CDR1 | | |
| SD- | WNDKRPS | 78 |
| 530939_VL_ | | |
| CDR2 | | |
| SD- | GGYDSSPGYVGI | 79 |
| 530939_VL_ | | |
| CDR3 | | |
| SD- | GCACTTGCGCTGACTCAGCCGTCCTCAGTGTCAGCAAACCCAGGA | 80 |
| 530939_VL_ | GAAACTGTCAAGATCACCTGCTCCGGGGGTTACAGCGGCTATGGC | |
| DNA | TGGTATCAGCAGAAGTCACCTGGCAGTGCCCCTGTCACAGTGATCT | |
| | ACTGGAACGACAAGAGACCCTCAAACATCCCTTCACGATTCTCCGG | |
| | TTCCCTATCCGGCTCCACAAACACATTAATCATCACTGGGGTCCAA | |
| | GCCGAGGACGAGGCTGTCTATTTCTGTGGTGGCTACGACAGCAGT | |
| | CCTGGTTATGTTGGTATATTTGGGGCCGGGACAACCCTGACCGTCC | |
| | TA | |
| SD- | AVTLDESGGGLQTPGGGLSLVCKASGFDFIDYDMFWVRQAPGKGLE | 81 |
| 589775_VH | WVGVINRSGSYTNYAPAVKGRATISRDNGQSTMRLQLNNLRAEDTG | |
| | TYYCARGACTHCWDADSAGYIDAWGHGTEVIVSS | |
| SD- | DYDMF | 82 |
| 589775_VH_ | | |
| CDR1 | | |
| SD- | VINRSGSYTNYAPAVKG | 83 |
| 589775_VH_ | | |
| CDR2 | | |
| SD- | GACTHCWDADSAGYIDA | 84 |
| 589775_VH_ | | |
| CDR3 | | |
| SD- | GCCGTGACGTTGGACGAGTCCGGGGGCGGCCTCCAGACGCCTGG | 85 |
| 589775_VH_ | AGGAGGGCTCAGCCTCGTCTGCAAGGCCTCCGGGTTCGACTTCAT | |
| DNA | CGATTATGACATGTTCTGGGTGCGACAGGCTCCAGGCAAGGGGCT | |
| | GGAGTGGGTCGGTGTTATTAACAGGAGTGGTAGTTACACAAACTA | |
| | CGCGCCGGCGGTGAAGGGCCGTGCCACCATCTCGAGGGACAACG | |
| | GGCAGAGCACGATGAGGCTGCAGCTGAACAACCTCAGGGCTGAG | |
| | GACACCGGCACCTACTACTGCGCCAGAGGTGCGTGTACTCATTGTT | |
| | GGGATGCAGACAGTGCTGGTTACATCGACGCATGGGGCCACGGG | |
| | ACCGAAGTCATCGTCTCCTCC | |
| SD- | ALALTQPSSVSANPGGTVKITCSGSSGSYGWHQQKSPGSAPVTVIYSS | 86 |
| 589775_VL | DKRPSDIPSRFSGALSGSTATLTITGVRAEDEAVYYCGGYDGSTDVGIF | |
| | GAGTTLTVL | |
| SD- | SGSSGSYG | 87 |
| 589775_VL_ | | |
| CDR1 | | |
| SD- | SSDKRPS | 88 |
| 589775_VL_ | | |
| CDR2 | | |
| SD- | GGYDGSTDVGI | 89 |
| 589775_VL_ | | |
| CDR3 | | |
| SD- | GCACTTGCGCTGACTCAGCCGTCCTCGGTGTCAGCAAACCCGGGA | 90 |
| 589775_VL_ | GGAACCGTCAAGATCACCTGCTCCGGGAGTAGTGGCAGTTATGGC | |
| DNA | TGGCACCAGCAGAAGTCACCTGGCAGTGCCCCTGTCACTGTGATCT | |
| | ATAGCAGCGACAAGAGACCCTCGGACATCCCTTCACGATTCTCCGG | |
| | TGCCCTATCCGGCTCCACAGCCACATTAACCATCACTGGGGTCCGA | |
| | GCCGAGGACGAGGCTGTCTATTACTGTGGTGGCTACGACGGCAGC | |
| | ACTGATGTTGGTATATTCGGGGCCGGGACAACCCTAACCGTCCTA | |
| SD- | QVQLVETGGDLVKPGGSLKLSCAASGFTFSTYGMSWVRQTPDKRLE | 91 |
| 661765_VH | WVATISSGGIYTYYSDSVKGRFTISRDNAKNTLYLQMSSLKSEDTAMY | |
| | YCARDGNYLGFDFWGQGTTLTVSS | |
| SD- | TYGMS | 92 |
| 661765_VH_ | | |
| CDR1 | | |
| SD- | TISSGGIYTYYSDSVKG | 93 |
| 661765_VH_ | | |
| CDR2 | | |
| SD- | DGNYLGFDF | 94 |
| 661765_VH_ | | |
| CDR3 | | |
| SD- | CAGGTGCAGCTTGTAGAGACCGGGGGAGACTTAGTGAAGCCTGG | 95 |
| 661765_VH_ | AGGGTCCCTGAAACTCTCCTGTGCAGCCTCTGGATTCACTTTCAGT | |
| DNA | ACCTATGGCATGTCTTGGGTTCGCCAGACTCCAGACAAGAGGCTG | |
| | GAGTGGGTCGCAACCATTAGTAGTGGTGGTATTTACACCTACTATT | |
| | CAGACAGTGTGAAGGGGCGATTCACCATCTCCAGAGACAATGCCA | |
| | AGAACACCCTGTACCTGCAAATGAGCAGTCTGAAGTCTGAGGACA | |
| | CAGCCATGTATTACTGTGCAAGAGATGGTAACTACCTGGGCTTTGA | |
| | CTTCTGGGGCCAAGGCACCACTCTCACAGTCTCCTCA | |
| SD- | DIVMTQSPASLAVSLGQRATISCRASESVESYGNSFMNWYQQKPGQ | 96 |
| 661765_VL | PPKLLIYRASNLESGIPARFSGSGSRTDFTLTINPVEADDVATYYCQQSY | |
| | EDPRTFGGGTKLEIK | |
| SD- | RASESVESYGNSFMN | 97 |
| 661765_VL_ | | |
| CDR1 | | |
| SD- | RASNLES | 98 |
| 661765_VL_ | | |
| CDR2 | | |
| SD- | QQSYEDPRT | 99 |
| 661765_VL_ | | |
| CDR3 | | |
| SD- | GACATTGTGATGACACAGTCTCCAGCTTCTTTGGCTGTGTCTCTAG | 100 |
| 661765_VL | GGCAGAGGGCCACCATATCCTGCAGAGCCAGTGAAAGTGTTGATA | |
| DNA | GTTATGGCAATAGTTTTATGCACTGGTACCAGCAGAAACCAGGAC | |
| | AGCCACCCAAACTCCTCATCTATCGTGCATCCAACCTAGAATCTGG | |
| | GATCCCTGCCAGGTTCAGTGGCAGTGGGTCTAGGACAGACTTCAC | |
| | CCTCACCATTAATCCTGTGGAGGCTGATGATGTTGCAACCTATTAC | |
| | TGTCAGCAAAGTAATGAGGATCCGTGGACGTTCGGTGGAGGCACC | |
| | AAGCTGGAAATCAAA | |
| SD- | AVTLDESGGGLQTPGGALSLVCKASGFDFIDYDMFWVRQAPGKGLE | 101 |
| 685208_VH | WVGVINRSGSYTNYAPAVKGRATISRDNGQSTMRLQLNNLRAEDTG | |
| | TYYCARGACTHCWDADSAGYIDAWGHGTEVIVSS | |
| SD- | DYDMF | 102 |
| 685208_VH_ | | |
| CDR1 | | |
| SD- | VINRSGSYTNYAPAVKG | 103 |
| 685208_VH_ | | |
| CDR2 | | |
| SD- | GACTHCWDADSAGYIDA | 104 |
| 685208_VH_ | | |
| CDR3 | | |
| SD- | GCCGTGACGTTGGACGAGTCCGGGGGGGGCCTCCAGACGCCCGG | 105 |
| 685208_VH_ | AGGAGCGCTCAGCCTCGTCTGCAAGGCCTCCGGGTTCGACTTCATC | |
| DNA | GATTATGACATGTTCTGGGTGCGACAGGCTCCAGGCAAGGGGCTG | |
| | GAGTGGGTCGGTGTTATTAACAGGAGTGGTAGTTACACAAACTAC | |
| | GCGCCGGCGGTGAAGGGCCGTGCCACCATCTCGAGGGACAACGG | |
| | GCAGAGCACAATGAGGCTGCAGCTGAACAACCTCAGGGCTGAGG | |
| | ACACCGGCACCTACTACTGCGCCAGAGGTGCGTGTACTCATTGTTG | |
| | GGATGCAGACAGTGCTGGTTACATCGACGCATGGGGCCACGGGA | |
| | CCGAAGTCATCGTCTCCTCC | |
| SD- | ALALTQPSSVSANPGETVKITCSGSSGSYGWFQQKSPGSAPVTVIYW | 106 |
| 685208_VL | NDKRPSDIPSRFSGSTSGSTGTLTITGVQADDEAVYFCGNADSSGTFG | |
| | AGTTLTVL | |
| SD- | SGSSGSYG | 107 |
| 685208_VL_ | | |
| CDR1 | | |
| SD- | WNDKRPS | 108 |
| 685208_VL_ | | |
| CDR2 | | |
| SD- | GNADSSGT | 109 |
| 685208_VL_ | | |
| CDR3 | | |
| SD- | GCACTTGCGCTGACTCAGCCGTCCTCGGTGTCAGCGAACCCGGGA | 110 |
| 685208_VL_ | GAAACCGTCAAGATCACCTGCTCCGGGAGTAGTGGCAGCTATGGC | |
| DNA | TGGTTCCAGCAGAAGTCACCTGGCAGTGCCCCTGTCACTGTGATCT | |
| | ATTGGAATGACAAGAGACCCTCGGACATCCCTTCACGATTCTCCGG | |
| | TTCCACATCCGGCTCCACGGGCACATTAACCATCACTGGGGTCCAA | |
| | GCCGACGACGAGGCTGTCTATTTCTGTGGGAATGCAGACAGCAGC | |
| | GGTACATTTGGGGCCGGGACAACCCTGACCGTCCTA | |
| SD- | AVTLDESGGGLQTPGGGLSLVCKASGFDFIDYDMFWVRQAPGKGLE | 111 |
| 802432_VH | WVAVINRSGSYTNYAPAVKGRATISRDNGQSTLRLQLNNLRAEDTGT | |
| | YYCARGACTHCWDADSAGYIDTWGHGTEVIVSS | |
| SD- | DYDMF | 112 |
| 802432_VH_ | | |
| CDR1 | | |
| SD- | VINRSGSYTNYAPAVKG | 113 |
| 802432_VH_ | | |
| CDR2 | | |
| SD- | GACTHCWDADSAGYIDT | 114 |
| 802432_VH_ | | |
| CDR3 | | |
| SD- | GCCGTGACGTTGGACGAGTCCGGGGGCGGCCTCCAGACGCCTGG | 115 |
| 802432_VH_ | AGGAGGGCTCAGCCTCGTCTGCAAGGCCTCCGGGTTCGACTTCAT | |
| DNA | CGATTATGACATGTTCTGGGTGCGACAGGCTCCAGGCAAGGGGCT | |
| | GGAGTGGGTCGCTGTTATTAACAGGAGTGGTAGTTACACAAACTA | |
| | CGCGCCGGCGGTGAAGGGCCGTGCCACCATCTCGAGGGACAACG | |
| | GGCAGAGCACACTGAGGCTGCAGCTGAACAACCTCAGGGCTGAG | |
| | GACACCGGCACCTACTACTGCGCCAGAGGTGCGTGTACTCATTGTT | |
| | GGGATGCAGACAGTGCTGGTTACATCGACACATGGGGCCACGGG | |
| | ACCGAAGTCATCGTCTCCTCC | |
| SD- | ALALTQPSSVSANPGETVKITCSGGSGSYGWFQQKSPGIAPVIVIFND | 116 |
| 802432_VL | DNRPSDIPSRFSGSKSGSTGTLTITGVQAEDEAVYFCGGYDGSTDAGIF | |
| | GAGTTLTVL | |
| SD- | SGGSGSYG | 117 |
| 802432_VL | | |
| CDR1 | | |
| SD- | NDDNRPS | 118 |
| 802432_VL_ | | |
| CDR2 | | |
| SD- | GGYDGSTDAGI | 119 |
| 802432_VL_ | | |
| CDR3 | | |
| SD- | GCACTTGCGCTGACTCAGCCGTCCTCGGTGTCAGCAAACCCAGGA | 120 |
| 802432_VL_ | GAAACCGTCAAGATCACCTGCTCCGGGGGTAGTGGCAGTTATGGC | |
| DNA | TGGTTCCAGCAGAAGTCTCCTGGCATTGCCCCTGTCATTGTGATCTT | |
| | TAATGACGACAACAGACCGTCGGACATCCCTTCACGATTCTCCGGT | |
| | TCCAAATCCGGCTCCACGGGCACATTAACCATCACTGGGGTCCAAG | |
| | CCGAAGACGAGGCTGTCTATTTCTGTGGTGGCTACGACGGCAGTA | |
| | CTGATGCTGGCATATTTGGGGCCGGGACAACCCTGACCGTCCTA | |
| SD- | EVKLDESGGGLVKPGGSLKLSCAASGFTFSSYTMSWVRQTPEKRLEW | 121 |
| 813149_VH | VATINSDGIYTYYPDSVKGRFTISRDNAKNTLYLQMSSLKSEDTAMYYC | |
| | TRVHYYGYDAMDYWGQGTSVTVSS | |
| SD- | SYTMS | 122 |
| 813149_VH_ | | |
| CDR1 | | |
| SD- | TINSDGIYTYYPDSVKG | 123 |
| 813149_VH_ | | |
| CDR2 | | |
| SD- | VHYYGYDAMDY | 124 |
| 813149_VH_ | | |
| CDR3 | | |
| SD- | GAAGTGAAGCTTGATGAGTCTGGGGGAGGCTTAGTGAAGCCTGG | 125 |
| 813149_VH_ | AGGGTCCCTGAAACTCTCCTGTGCAGCCTCTGGATTCACTTTCAGT | |
| DNA | AGCTATACCATGTCTTGGGTTCGCCAGACTCCGGAGAAGAGGCTG | |
| | GAGTGGGTCGCAACTATTAATAGTGATGGTATTTACACCTACTATC | |
| | CAGACAGTGTGAAGGGCCGATTCACCATCTCCAGAGACAATGCCA | |
| | AGAACACCCTGTACCTGCAAATGAGCAGTCTGAAGTCTGAGGACA | |
| | CAGCCATGTATTACTGTACAAGAGTACATTACTACGGCTACGATGC | |
| | TATGGACTACTGGGGTCAAGGAACCTCAGTCACCGTCTCCTCA | |
| SD- | EIQMTQSPASLSASVGETVTITCRASENIYSYLAWYQQKQGKSPQLLV | 126 |
| 813149_VL | YSAKTLAEGVPSRFSGSGSGTHFSLKINSLQPEDFGSYYCQHHYGIPLTF | |
| | GAGTKLELK | |
| SD- | RASENIYSYLA | 127 |
| 813149_VL_ | | |
| CDR1 | | |
| SD- | SAKTLAE | 128 |
| 813149_VL | | |
| CDR2 | | |
| SD- | QHHYGIPLT | 129 |
| 813149_VL | | |
| CDR3 | | |
| SD- | GAAATCCAGATGACCCAGTCTCCAGCCTCCCTATCTGCATCTGTGG | 130 |
| 813149_VL | GAGAAACTGTCACCATCACATGTCGAGCAAGTGAGAATATTTACA | |
| DNA | GTTATTTAGCATGGTATCAGCAGAAACAGGGAAAATCTCCTCAGCT | |
| | CCTGGTCTATAGTGCAAAAACCTTAGCAGAAGGTGTGCCATCAAG | |
| | GTTCAGTGGCAGTGGATCAGGCACACATTTTTCTCTGAAGATCAAC | |
| | AGCCTGCAGCCTGAAGATTTTGGGAGTTATTACTGTCAACATCATT | |
| | ATGGTATTCCGCTCACGTTCGGTGCTGGGACCAAGCTGGAGCTGA | |
| | AA | |
| SD- | AVTLDESGGGLQTPGGALSLVCKGSGFTFSSYAMGWVRQAPGKGLE | 131 |
| 970200_VH | YVAAISSGSSTGYGAAVKGRATISKDTGQSTVRLQLNNLRAEDTATYY | |
| | CAKSGYGGSASYVSDIDAWGHGTEVIVSS | |
| SD- | SYAMG | 132 |
| 970200_VH_ | | |
| CDR1 | | |
| SD- | AISSGSSTGYGAAVKG | 133 |
| 970200_VH_ | | |
| CDR2 | | |
| SD- | SGYGGSASYVSDIDA | 134 |
| 970200_VH_ | | |
| CDR3 | | |
| SD- | GCCGTGACGTTGGACGAGTCCGGGGGCGGCCTCCAGACGCCCGG | 135 |
| 970200_VH_ | AGGAGCGCTCAGCCTCGTCTGCAAGGGCTCCGGGTTCACCTTCAG | |
| DNA | CAGTTATGCCATGGGTTGGGTGCGCCAGGCGCCCGGCAAGGGGTT | |
| | GGAATATGTCGCAGCTATTAGCAGTGGTAGTAGCACAGGATACGG | |
| | GGCAGCGGTGAAGGGCCGTGCCACCATCTCGAAGGACACCGGGC | |
| | AGAGCACAGTGAGGCTGCAGCTGAACAACCTCAGGGCTGAGGAC | |
| | ACCGCCACCTACTACTGCGCCAAAAGTGGTTATGGTGGTAGTGCG | |
| | AGCTATGTTAGCGATATCGACGCATGGGGCCACGGGACCGAAGTC | |
| | ATCGTCTCCTCC | |
| SD- | ALALTQPSSVSANPGETVEITCSGGGDYYGTYYYGWYQQKAPGSAPV | 136 |
| 970200_VL | TVIYDNTKRPSNIPSRFSGSTSGSTNTLTITGVQADDEAVYYCGSYDSS | |
| | AGIFGAGTTLTVL | |
| SD- | SGGGDYYGTYYYG | 137 |
| 970200_VL_ | | |
| CDR1 | | |
| SD- | DNTKRPS | 138 |
| 970200_VL_ | | |
| CDR2 | | |
| SD- | GSYDSSAGI | 139 |
| 970200_VL_ | | |
| CDR3 | | |
| SD- | GCACTTGCGCTGACTCAGCCGTCCTCGGTGTCAGCGAACCCGGGA | 140 |
| 970200_VL_ | GAAACCGTTGAGATCACCTGCTCCGGGGGTGGCGACTACTATGGA | |
| DNA | ACTTACTATTATGGCTGGTACCAGCAGAAGGCACCTGGCAGTGCCC | |
| | CTGTCACTGTGATCTATGACAACACCAAGAGACCCTCGAACATCCC | |
| | TTCACGATTCTCCGGTTCCACATCCGGCTCCACAAACACATTAACCA | |
| | TCACTGGGGTCCAAGCCGACGACGAGGCTGTCTATTACTGTGGGA | |
| | GCTACGACAGCAGTGCTGGTATATTTGGGGCCGGGACAACCCTGA | |
| | CCGTCCTA | |
| huTROP2 | MARGPGLAPPPLRLPLLLLVLAAVTGHTAAQDNCTCPTNKMTVCSPD | 141 |
| | GPGGRCQCRALGSGMAVDCSTLTSKCLLLKARMSAPKNARTLVRPSE | |
| | HALVDNDGLYDPDCDPEGRFKARQCNQTSVCWCVNSVGVRRTDKG | |
| | DLSLRCDELVRTHHILIDLRHRPTAGAFNHSDLDAELRRLFRERYRLHP | |
| | KFVAAVHYEQPTIQIELRQNTSQKAAGDVDIGDAAYYFERDIKGESLF | |
| | QGRGGLDLRVRGEPLQVERTLIYYLDEIPPKFSMKRLTAGLIAVIVVVV | |
| | VALVAGMAVLVITNRRKSGKYKKVEIKELGELRKEPSL | |
| msTROP2 | MARGLDLAPLLLLLLAMATRFCTAQSNCTCPTNKMTVCDTNGPGGV | 142 |
| | CQCRAMGSQVLVDCSTLTSKCLLLKARMSARKSGRSLVMPSEHAILD | |
| | NDGLYDPECDDKGRFKARQCNQTSVCWCVNSVGVRRTDKGDQSLR | |
| | CDEVVRTHHILIELRHRPTDRAFNHSDLDSELRRLFQERYKLHPSFLSAV | |
| | HYEEPTIQIELRQNASQKGLRDVDIADAAYYFERDIKGESLFMGRRGL | |
| | DVQVRGEPLHVERTLIYYLDEKPPQFSMKRLTAGVIAVIAVVSVAVVA | |
| | GVVVLVVTKRRKSGKYKKVELKELGEMRSEPSL | |
| cyTROP2 | MARGPGLAPPPLRLPLLLLLLAAVTGHTAAQDNCTCPTNKMTVCSPD | 143 |
| | GPGGRCQCRALGSGVAVDCSTLTSKCLLLKARMSAPKNARTLVRPNE | |
| | HALVDNDGLYDPDCDPEGRFKARQCNQTSVCWCVNSVGVRRTDKG | |
| | DLSLRCDELVRTHHILIDLRHRPTAGAFNHSDLDAELRRLFRERYRLHP | |
| | KFVAAVHYEQPTIQIELRQNTSQKAAGDVDIGDAAYYFERDVKGESLF | |
| | QGRGGLDLRVRGEPLQVERTLIYYLDEIPPKFSMKRLTAGLIAVIVVVV | |
| | VALVAGVAVLVISNRRKSGKYKKVEIKELGELRKEPSL | |
| EPCAM | MAPPQVLAFGLLLAAATATFAAAQEECVCENYKLAVNCFVNNNRQC | 144 |
| | QCTSVGAQNTVICSKLAAKCLVMKAEMNGSKLGRRAKPEGALQNND | |
| | GLYDPDCDESGLFKAKQCNGTSMCWCVNTAGVRRTDKDTEITCSER | |
| | VRTYWIIIELKHKAREKPYDSKSLRTALQKEITTRYQLDPKFITSILYENN | |
| | VITIDLVQNSSQKTQNDVDIADVAYYFEKDVKGESLFHSKKMDLTVNG | |
| | EQLDLDPGQTLIYYVDEKAPEFSMQGLKAGVIAVIVVVVIAVVAGIVVL | |
| | VISRKKRMAKYEKAEIKEMGEMHRELNA | |
| hulgG1 | ASTKGPSVFPLAPSSKSTSGGTAALGCLVKDYFPEPVTVSWNSGALTS | 145 |
| | GVHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| huLCkappa | RTVAAPSVFIFPPSDEQLKSGTASVVCLLNNFYPREAKVQWKVDNAL | 146 |
| | QSGNSQESVTEQDSKDSTYSLSSTLTLSKADYEKHKVYACEVTHQGLS | |
| | SPVTKSFNRGEC | |
| huLClambda | GQPKAAPSVTLFPPSSEELQANKATLVCLISDFYPGAVTVAWKADSSP | 147 |
| | VKAGVETTTPSKQSNNKYAASSYLSLTPEQWKSHRSYSCQVTHEGSTV | |
| | EKTVAPTECS | |
| Peptide1 | MTVCSPDGPGGRCQC | 148 |
| Peptide2 | PDGPGGRCQCRALGS | 149 |
| Peptide3 | DLDAELRRLFRERYR | 150 |
| Peptide4 | LRRLFRERYRLHPKF | 151 |
| Peptide5 | RERYRLHPKFVAAVH | 152 |
| Peptide6 | LHPKFVAAVHYEQPT | 153 |
| Peptide7 | DVDIGDAAYYFERDI | 154 |
| Peptide8 | DAAYYFERDIKGESL | 155 |

### Additional Sequences

| Sequence | | SEQ |
|---|---|---|
| descrip- | | ID |
| tion | Sequence | NO: |
| 589-VH_1 | QVQLLESGGGLVKPGGSLRLSCAASGFTFSDYDMFWIRQAPGKGLEWVSV | 156 |
| | INRSGSYTNYAPAVKGRFTISRDNAKNSLYLQMNSLRAEDTAVYYCARGAC | |
| | THCWDADSAGYIDAWGQGTLVTVSS | |
| 589-VH_1 | CAGGTACAGCTGTTGGAAAGTGGAGGAGGGCTGGTGAAGCCTGGTGG | 157 |
| DNA | CAGCCTGCGGCTGAGCTGCGCCGCCAGCGGATTTACCTTCAGTGACTAT | |
| | GATATGTTCTGGATCCGGCAGGCCCCCGGGAAAGGTTTGGAATGGGTT | |
| | TCTGTGATTAACCGCAGTGGCAGCTATACAAATTACGCCCCGGCGGTGA | |
| | AGGGGAGGTTTACCATAAGCAGGGACAATGCGAAGAACTCTTTATACC | |
| | TGCAAATGAACAGCCTGAGGGCGGAAGATACGGCAGTTTACTATTGCG | |
| | CACGAGGTGCTTGTACTCATTGTTGGGATGCTGACTCTGCTGGCTATAT | |
| | TGACGCGTGGGGCCAGGGGACACTGGTGACAGTTAGCTCT | |
| 589-VH_2 | QVQLLESGGGLVKPGGSLRLSCAASGFDFIDYDMFWIRQAPGKGLEWVG | 158 |
| (SD- | VINRSGSYTNYAPAVKGRATISRDNAKNSMYLQMNSLRAEDTAVYYCARG | |
| 861408_V | ACTHCWDADSAGYIDAWGQGTLVTVSS | |
| H) | | |
| 589-VH_2 | CAAGTACAGTTGCTGGAGTCAGGGGGTGGGTTAGTGAAGCCAGGAGG | 159 |
| DNA | CAGCCTTCGCTTGTCCTGCGCAGCCTCTGGTTTTGATTTCATCGACTACG | |
| | ATATGTTCTGGATCCGGCAGGCCCCTGGGAAAGGGCTGGAGTGGGTAG | |
| | GCGTCATCAACAGGTCCGGTTCTTATACCAATTACGCCCCTGCCGTAAA | |
| | GGGAAGAGCTACGATCAGTCGCGACAACGCGAAAAACTCTATGTATCT | |
| | CCAAATGAATTCACTGCGGGCAGAAGACACAGCCGTGTATTACTGTGC | |
| | GCGGGGCGCGTGTACGCATTGCTGGGATGCAGACTCCGCCGGATACAT | |
| | CGATGCTTGGGGGCAGGGAACTTTGGTAACCGTGAGCTCC | |
| 589-VH_3 | QVQLLESGGGLVKPGGSLRLSCAASGFDFIDYDMFWIRQAPGKGLEWVG | 160 |
| | VINRSGSYTNYAPAVKGRATISRDNGQSTMYLQMNSLRAEDTAVYYCARG | |
| | ACTHCWDADSAGYIDAWGQGTLVTVSS | |
| 589-VH_3 | CAGGTACAATTGCTGGAAAGTGGCGGAGGGCTGGTGAAACCTGGAGG | 161 |
| DNA | GAGTCTGCGGCTCAGTTGTGCAGCTTCTGGATTCGACTTTATCGACTAT | |
| | GATATGTTCTGGATCCGCCAGGCCCCGGGCAAAGGCCTCGAGTGGGTT | |
| | GGCGTGATCAATAGATCAGGTAGCTATACAAACTACGCCCCTGCGGTG | |
| | AAGGGCAGGGCCACCATTAGTCGGGACAACGGACAGTCAACAATGTAC | |
| | CTTCAGATGAACTCTTTGAGGGCCGAGGATACGGCCGTCTACTATTGCG | |
| | CTCGCGGCGCATGTACGCATTGCTGGGACGCGGATAGTGCTGGCTATA | |
| | TCGATGCGTGGGGCCAGGGCACCTTGGTCACAGTGAGCTCT | |
| 589-VH_4 | EVQLVESGGGLVQPGGSLRLSCAASGFTFSSYWMHWVRQAPGKGLVWV | 162 |
| | SRINSDGSSTSYADSVKGRFTISRDNAKNTLYLQMNSLRAEDTAVYYCARG | |
| | APTYCSGGSCYGYFDYWGQGTLVTVSS | |
| 589-VH_4 | GAGGTACAGCTCGTTGAGTCAGGGGGGGGCTTAGTGCAGCCAGGCGG | 163 |
| DNA | CTCTTTAAGACTCTCATGCGCCGCCAGTGGCTTCACATTTAGCTCCTATT | |
| | GGATGCACTGGGTGCGTCAAGCTCCCGGCAAAGGCCTGGTCTGGGTTT | |
| | CCCGCATCAACAGCGATGGCAGTTCCACGTCCTATGCAGACTCAGTTAA | |
| | GGGTCGGTTTACTATCTCACGGGACAACGCAAAGAACACCCTGTATCTG | |
| | CAGATGAACTCACTGCGCGCAGAGGATACTGCCGTGTACTATTGTGCAA | |
| | GGGGGGCTCCCACCTACTGTAGCGGCGGCAGCTGCTATGGCTACTTCG | |
| | ACTATTGGGGTCAAGGGACCCTGGTTACAGTAAGCTCC | |
| 589-VL_1 | SYELTQPPSVSVSPGQTASITCSGSSGSYGWYQQKPGQSPVLVIYSSDKRPS | 164 |
| | GIPERFSGSNSGNTATLTISGTQAMDEADYYCGGYDGSTDVGIFGGGTKLT | |
| | VL | |
| 589-VL_1 | TCTTACGAGCTGACCCAGCCGCCGTCAGTCTCTGTCAGTCCCGGCCAGA | 165 |
| DNA | CCGCTAGCATTACCTGTTCTGGCTCCAGCGGATCTTATGGGTGGTATCA | |
| | GCAGAAGCCTGGACAGTCTCCTGTCCTTGTCATATACAGCTCAGACAAG | |
| | CGACCGTCAGGCATACCGGAGAGGTTCAGCGGCTCAAATTCAGGCAAT | |
| | ACCGCCACACTCACAATCAGTGGGACTCAGGCCATGGATGAGGCGGAC | |
| | TACTATTGCGGCGGCTACGATGGGTCAACCGATGTGGGTATCTTCGGC | |
| | GGCGGCACAAAGCTGACCGTGTTG | |
| 589-VL_2 | SLELTQPPSVSVSPGQTASITCSGSSGSYGWHQQKPGQSPVTVIYSSDKRPS | 166 |
| (SD- | GIPERFSGSSSGSTATLTISGTQAMDEADYYCGGYDGSTDVGIFGGGTKLTV | |
| 861408_V | L | |
| L) | | |
| 589-VL_2 | TCACTCGAGCTTACGCAGCCCCCCAGCGTTAGTGTGAGCCCTGGCCAAA | 167 |
| DNA | CCGCAAGCATCACTTGCAGCGGATCCTCCGGGTCCTACGGCTGGCATCA | |
| | GCAGAAGCCCGGGCAGTCTCCCGTTACAGTTATATACTCATCTGATAAA | |
| | CGTCCATCCGGGATCCCGGAAAGGTTCTCAGGCAGCTCAAGTGGATCA | |
| | ACCGCTACCTTGACTATATCTGGGACGCAAGCAATGGATGAGGCAGAT | |
| | TACTATTGTGGGGGTTACGACGGGTCTACCGACGTGGGTATTTTTGGCG | |
| | GCGGCACCAAGCTTACCGTACTT | |
| 589-VL_3 | QSALTQPPSVSASPGQTVTITCSGSSGSYGSWYQQKPGQAPVLVIYEDSKR | 168 |
| | PSGIPDRFSGSKSGSTATLTISGVQAEDEAVYYCQGYDGSTDVGIFGGGTKL | |
| | TVL | |
| 589-VL_3 | CAGAGCGCTCTGACTCAGCCCCCCAGTGTGTCTGCAAGCCCTGGGCAAA | 169 |
| DNA | CAGTGACTATCACATGTTCCGGTTCCTCCGGGAGTTATGGGAGCTGGTA | |
| | CCAACAGAAGCCAGGGCAAGCCCCAGTCCTTGTAATTTATGAGGACAG | |
| | TAAGAGACCCTCTGGGATCCCCGATCGGTTTAGTGGCAGCAAAAGCGG | |
| | GAGCACAGCAACACTGACTATCAGCGGAGTTCAAGCAGAGGATGAAGC | |
| | CGTGTACTACTGCCAGGGGTATGACGGTAGTACTGACGTTGGGATATTC | |
| | GGAGGAGGGACCAAGCTCACCGTCCTG | |
| aHEL VH | DVQLQESGPSLVKPSQTLSLTCSVTGDSITSDYWSWIRKFPGNRLEYMGYV | 170 |
| | SYSGSTYYNPSLKSRISITRDTSKNQYYLDLNSVTTEDTATYYCANWDGDYW | |
| | GQGTLVTVSA | |
| aHEL VH | GACGTACAATTGCAAGAATCAGGCCCCTCACTTGTGAAACCGTCACAAA | 171 |
| DNA | CTCTTTCACTTACATGTTCCGTGACAGGTGATAGTATTACCTCCGATTAT | |
| | TGGTCATGGATACGAAAATTCCCTGGAAATCGGCTGGAGTACATGGGT | |
| | TACGTTTCTTATTCCGGCTCTACTTATTACAATCCCAGTTTGAAATCTAGG | |
| | ATTAGCATCACGAGAGATACATCTAAGAACCAGTATTATCTTGATCTGA | |
| | ATAGCGTTACTACGGAGGACACAGCCACGTATTATTGTGCAAATTGGG | |
| | ACGGTGATTACTGGGGTCAGGGTACACTGGTAACGGTCTCAGCG | |
| aHEL VL | DIVLTQSPATLSVTPGNSVSLSCRASQSIGNNLHWYQQKSHESPRLLIKYAS | 172 |
| | QSISGIPSRFSGSGSGTDFTLSINSVETEDFGMYFCQQSNSWPYTFGGGTKL | |
| | EIK | |
| aHEL VL | GACATCGTACTTACCCAATCTCCAGCGACATTGTCAGTAACACCAGGAA | 173 |
| DNA | ACAGCGTCTCTCTGTCATGTAGAGCCTCACAATCTATAGGAAATAATCT | |
| | GCATTGGTATCAGCAGAAGTCCCACGAGTCACCGAGGTTGCTTATAAA | |
| | GTATGCCTCCCAGTCTATTTCCGGGATACCATCCAGATTCAGTGGATCTG | |
| | GTTCCGGTACGGATTTCACCCTGAGTATCAATAGCGTAGAAACAGAAGA | |
| | TTTCGGTATGTATTTTTGTCAACAGAGTAACTCCTGGCCCTACACATTCG | |
| | GAGGAGGAACGAAACTCGAGATCAAG | |
| CD3 Gen 1 | EVQLVESGGGLVQPGGSLRLSCAASGFTFNTYAMNWVRQAPGKGLEWV | 174 |
| VH | GRIRSKYNNYATYYADSVKGRFTISRDDSKNTLYLQMNSLRAEDTAVYYCV | |
| | RHGNFGNSYVSWFAYWGQGTLVTVSS | |
| CD3 Gen 1 | GAAGTGCAGCTTGTTGAAAGCGGCGGCGGGTTGGTGCAGCCAGGCGG | 175 |
| VH DNA | CAGTCTGCGGCTGTCATGTGCTGCTTCAGGCTTCACCTTCAATACATATG | |
| | CAATGAATTGGGTGCGCCAAGCGCCTGGCAAGGGTTTAGAATGGGTGG | |
| | GAAGGATCCGCAGCAAGTATAACAATTATGCGACATATTACGCGGATTC | |
| | TGTCAAGGGGAGATTCACAATCTCTCGGGACGATTCAAAAAACACTTTA | |
| | TACCTTCAAATGAACTCACTCAGGGCCGAAGATACTGCTGTCTATTACT | |
| | GTGTCCGACATGGTAACTTTGGCAACAGTTACGTGTCATGGTTCGCTTA | |
| | TTGGGGACAAGGAACGCTGGTTACTGTCTCCTCA | |
| CD3 Gen 1 | DIQMTQSPSSLSASVGDRVTITCRSSTGAVTTSNYANWVQQKPGKAPKGLI | 176 |
| VL | GGTNKRAPGVPARFSGSGSGTDFTLTISSLQPEDFATYYCALWYSNLWVFG | |
| | QGTKVEIK | |
| CD3 Gen 1 | GACATACAAATGACTCAATCACCGTCTTCTTTGTCCGCGAGCGTGGGAG | 177 |
| VL DNA | ATCGGGTCACTATAACGTGCCGAAGCTCCACCGGCGCTGTAACCACTTC | |
| | CAACTATGCTAATTGGGTGCAGCAAAAGCCGGGGAAGGCACCTAAAGG | |
| | CCTTATCGGGGGAACAAACAAGCGCGCACCTGGAGTTCCAGCTCGGTT | |
| | CAGCGGTTCAGGGTCAGGAACCGACTTTACGTTGACGATTAGTTCTCTT | |
| | CAGCCCGAAGATTTTGCCACCTATTACTGTGCCCTTTGGTATTCCAATTT | |
| | GTGGGTGTTTGGGCAGGGAACAAAGGTCGAAATTAAA | |
| SD-385483 | EVQLVESGGGLVQPGGSLRLSCAASGFDFNAYAMNWVRQAPGKGLEWV | 178 |
| VH | GRIRSKYNNYATYYADSVKGRFTISRDDSKNTLYLQMNSLRAEDTAVYYCV | |
| | RHGNFGNSYVSWFAYWGQGTLVTVSS | |
| SD-385483 | GAGGTCCAACTCGTGGAATCAGGCGGGGGATTAGTGCAGCCTGGCGG | 179 |
| VH DNA | TTCCCTTAGGCTCTCGTGTGCTGCATCTGGCTTCGACTTTAATGCCTACG | |
| | CCATGAACTGGGTGCGTCAGGCACCCGGAAAGGGATTGGAGTGGGTC | |
| | GGACGCATTCGCAGCAAGTACAATAACTACGCCACTTACTACGCTGATT | |
| | CCGTGAAAGGCCGATTCACAATCAGCCGGGACGATAGCAAAAACACCC | |
| | TGTATCTGCAGATGAATAGTCTGAGGGCCGAAGACACAGCCGTGTACT | |
| | ATTGCGTTCGGCACGGGAACTTTGGCAATTCTTATGTATCCTGGTTCGCC | |
| | TACTGGGGGCAGGGCACTCTGGTCACCGTTTCTAGT | |
| SD-385483 | DIQMTQSPSSLSASVGDRVTITCRSSTGAVTTSNYANWVQQKPGKAPKGLI | 180 |
| VL | GGTNKRAPGVPARFSGSGSGTDFTLTISSLQPEDFATYYCAAWHDNAWVF | |
| | GQGTKVEIK | |
| SD-385483 | GATATACAAATGACACAGTCTCCCTCTTCCCTCTCTGCCTCAGTCGGCGA | 181 |
| VL DNA | TCGCGTGACAATTACATGTAGATCCTCCACTGGTGCCGTTACCACATCCA | |
| | ACTACGCTAACTGGGTACAGCAGAAGCCTGGCAAGGCACCCAAAGGCC | |
| | TTATTGGCGGGACTAATAAACGGGCACCTGGAGTCCCAGCCAGGTTCTC | |
| | GGGAAGTGGGAGCGGGACTGACTTTACCCTGACCATCTCAAGCTTACA | |
| | GCCAGAGGACTTCGCTACGTATTACTGCGCCGCATGGCACGACAACGCT | |
| | TGGGTGTTTGGGCAAGGGACCAAGGTGGAGATCAAA | |
| IgG1 hole | ASTKGPSVFPLAPSSKSTSGGTAALGCLVKDYFPEPVTVSWNSGALTSGVH | 182 |
| | TFPAVLQSSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVDVSHEDPE | |
| | VKFNWYVDGVEVHNAKTKPREEQYASTYRVVSVLTVLHQDWLNGKEYKC | |
| | KVSNKALPAPIEKTISKAKGQPREPQVYTLPPSREEMTKNQVSLTCLVKGFY | |
| | PSDIAVEWESNGQPENNYKTTPPVLDSDGSFLLYSKLTVDKSRWQQGNVF | |
| | SCSVMHEALHNHYTQKSLSLSPGK | |
| IgG1 hole | GCATCCACCAAGGGCCCcagcgtgttccccctggcccccagcagcaagagcaccagc | 183 |
| DNA | ggcggcaccgccgccctgggctgcctggtgaaggactacttccccgagcccgtgaccgtgagc | |
| | tggaacagcggcgccctgaccagcggcgtgcacaccttccccgccgtgctgcagagcagcgg | |
| | cctgtacagcctgagcagcgtggtgaccgtgcccagcagcagcctgggcacccagacctacat | |
| | ctgcaacgtgaaccacaagcccagcaacaccaaggtggacaagagagtggagcccaagagc | |
| | tgcgacaagacccacacctgccccccctgccccgcccccgagctgctgggcggccccagcgtg | |
| | ttcctgttcccccccaagcccaaggacaccctgatgatcagcagaacccccgaggtgacctgc | |
| | gtggtggtggacgtgagccacgaggaccccgaggtgaagttcaactggtacgtggacggcgt | |
| | ggaggtgcacaacgccaagaccaagcccagagaggagcagtacgcgagcacctacagagt | |
| | ggtgagcgtgctgaccgtgctgcaccaggactggctgaacggcaaggagtacaagtgcaagg | |
| | tgagcaacaaggccctgcccgcccccatcgagaagaccatcagcaaggccaagggccagcc | |
| | cagagagccccaggtgtacaccctgccccccagcagagaggagatgaccaagaaccaggtg | |
| | agcctgacctgcctggtgaagggcttctaccccagcgacatcgccgtggagtgggagagcaac | |
| | ggccagcccgagaacaactacaagaccaccccccccgtgctggacagcgacggcagcttcct | |
| | gctgtacagcaagctgaccgtggacaagagcagatggcagcagggcaacgtgttcagctgca | |
| | gcgtgatgcacgaggccctgcacaaccactacacccagaagagcctgagcctgagccccggc | |
| | aa | |
| IgG1 knob | ASTKGPSVFPLAPSSKSTSGGTAALGCLVKDYFPEPVTVSWNSGALTSGVH | 184 |
| | TFPAVLQSSGLYSLSSVVTVPSSSLGTQTYICNVNHKPSNTKVDKRVEPKSC | |
| | DKTHTCPPCPAPELLGGPSVFLFPPKPKDTLMISRTPEVTCVVVDVSHEDPE | |
| | VKFNWYVDGVEVHNAKTKPREEQYASTYRVVSVLTVLHQDWLNGKEYKC | |
| | KVSNKALPAPIEKTISKAKGQPREPQVYTLPPSREEMTKNQVSLTCLVKGFY | |
| | PSDIAVEWESNGQPENNYKTTPPVLDSDGSFFLYSRLTVDKSRWQQGNVF | |
| | SCSVMHEALHNHYTQKSLSLSPGK | |
| IgG1 knob | GCGTCAACAAAAGGTCCCTCAGTGTTTCCTCTGGCCCCATCAAGCAAGA | 185 |
| DNA | GCACCTCCGGCGGCACCGCCGCACTGGGATGTCTCGTGAAGGATTACTT | |
| | CCCTGAGCCTGTAACTGTGAGCTGGAATTCTGGTGCGCTGACAAGTGGT | |
| | GTCCATACCTTCCCCGCCGTCTTGCAGAGCAGTGGGCTGTATAGCTTAA | |
| | GCAGCGTAGTGACCGTCCCTAGCTCCTCCCTGGGTACTCAGACTTATAT | |
| | CTGCAATGTCAATCATAAACCCTCTAATACCAAAGTCGATAAACGCGTG | |
| | GAGCCTAAATCTTGTGATAAAACCCATACCTGCCCACCATGTCCCGCCCC | |
| | TGAGCTGCTCGGCGGTCCATCCGTCTTCCTGTTTCCTCCAAAACCAAAG | |
| | GACACATTGATGATTAGTAGGACACCCGAGGTCACCTGTGTGGTGGTT | |
| | GACGTTTCCCACGAGGACCCAGAGGTCAAATTCAACTGGTATGTGGAC | |
| | GGCGTGGAGGTCCACAACGCTAAAACCAAACCCAGAGAAGAACAGTAC | |
| | GCCTCTACTTATAGAGTTGTGAGCGTCCTCACGGTGCTGCACCAGGATT | |
| | GGCTGAATGGCAAGGAGTATAAGTGCAAAGTCTCTAATAAAGCACTCC | |
| | CTGCGCCAATAGAGAAGACAATTAGCAAGGCAAAGGGACAACCACGC | |
| | GAACCTCAGGTGTACACTCTCCCCCCTAGCCGGGAAGAGATGACAAAA | |
| | AATCAGGTTTCTTTAACATGTCTGGTGAAAGGATTTTACCCTTCCGACAT | |
| | CGCGGTCGAATGGGAGTCAAATGGACAACCCGAGAATAATTACAAGAC | |
| | AACACCACCAGTGTTAGACTCCGATGGCTCCTTCTTTCTGTACAGCAGG | |
| | CTGACGGTGGACAAGAGCCGCTGGCAACAGGGGAATGTCTTCTCTTGC | |
| | TCTGTGATGCATGAGGCACTCCACAATCATTATACGCAGAAAAGTCTCT | |
| | CTCTCTCTCCAGGCAAA | |

| Name | Sequence |
|---|---|
| SD- | EVQLVESGGGLVQPGGSLRLSCAASGFTFNTYAMNWVRQAPGKGLEWVGRIRSKYNNYA |
| 17407 | TYYADSVKGRFTISRDDSKNTLYLQMNSLRAEDTAVYYCVRHGNFGNSYVSWFAYWGQG |
| 8_HC_ | TLVTVSSASTKGPSVFPLAPSSKSTSGGTAALGCLVKDYFPEPVTVSWNSGALTSGVHTFPA |
| knob | VLQSSGLYSLSSVVTVPSSSLGTQTYICNVNHKPSNTKVDKRVEPKSCDKTHTCPPCPAPELL |
| AA | GGPSVFLFPPKPKDTLMISRTPEVTCVVVDVSHEDPEVKFNWYVDGVEVHNAKTKPREEQ |
| | YASTYRVVSVLTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSRLTVDKSRW |
| | QQGNVFSCSVMHEALHNHYTQKSLSLSPGK (SEQ ID NO: 186) |
| SD- | GAAGTGCAGCTTGTTGAAAGCGGCGGCGGGTTGGTGCAGCCAGGCGGCAGTCTGCGG |
| 17407 | CTGTCATGTGCTGCTTCAGGCTTCACCTTCAATACATATGCAATGAATTGGGTGCGCCA |
| 8_HC_ | AGCGCCTGGCAAGGGTTTAGAATGGGTGGGAAGGATCCGCAGCAAGTATAACAATTA |
| knob | TGCGACATATTACGCGGATTCTGTCAAGGGGAGATTCACAATCTCTCGGGACGATTCAA |
| DNA | AAAACACTTTATACCTTCAAATGAACTCACTCAGGGCCGAAGATACTGCTGTCTATTACT |
| | GTGTCCGACATGGTAACTTTGGCAACAGTTACGTGTCATGGTTCGCTTATTGGGGACAA |
| | GGAACGCTGGTTACTGTCTCCTCAGCGTCAACAAAAGGTCCCTCAGTGTTTCCTCTGGC |
| | CCCATCAAGCAAGAGCACCTCCGGCGGCACCGCCGCACTGGGATGTCTCGTGAAGGAT |
| | TACTTCCCTGAGCCTGTAACTGTGAGCTGGAATTCTGGTGCGCTGACAAGTGGTGTCCA |
| | TACCTTCCCCGCCGTCTTGCAGAGCAGTGGGCTGTATAGCTTAAGCAGCGTAGTGACC |
| | GTCCCTAGCTCCTCCCTGGGTACTCAGACTTATATCTGCAATGTCAATCATAAACCCTCT |
| | AATACCAAAGTCGATAAACGCGTGGAGCCTAAATCTTGTGATAAAACCCATACCTGCCC |
| | ACCATGTCCCGCCCCTGAGCTGCTCGGCGGTCCATCCGTCTTCCTGTTTCCTCCAAAACC |
| | AAAGGACACATTGATGATTAGTAGGACACCCGAGGTCACCTGTGTGGTGGTTGACGTT |
| | TCCCACGAGGACCCAGAGGTCAAATTCAACTGGTATGTGGACGGCGTGGAGGTCCACA |
| | ACGCTAAAACCAAACCCAGAGAAGAACAGTACGCCTCTACTTATAGAGTIGTGAGCGT |
| | CCTCACGGTGCTGCACCAGGATTGGCTGAATGGCAAGGAGTATAAGTGCAAAGTCTCT |
| | AATAAAGCACTCCCTGCGCCAATAGAGAAGACAATTAGCAAGGCAAAGGGACAACCAC |
| | GCGAACCTCAGGTGTACACTCTCCCCCCTAGCCGGGAAGAGATGACAAAAAATCAGGT |
| | TTCTTTAACATGTCTGGTGAAAGGATTTTACCCTTCCGACATCGCGGTCGAATGGGAGT |
| | CAAATGGACAACCCGAGAATAATTACAAGACAACACCACCAGTGTTAGACTCCGATGG |
| | CTCCTTCTTTCTGTACAGCAGGCTGACGGTGGACAAGAGCCGCTGGCAACAGGGGAAT |
| | GTCTTCTCTTGCTCTGTGATGCATGAGGCACTCCACAATCATTATACGCAGAAAAGTCTC |
| | TCTCTCTCTCCAGGCAAA (SEQ ID NO: 187) |
| SD- | EVKLDESGGGLVKPGGSLKLSCAASGFTFSSYTMSWVRQTPEKRLEWVATINSDGIYTYYP |
| 17407 | DSVKGRFTISRDNAKNTLYLQMSSLKSEDTAMYYCTRVHYYGYDAMDYWGQGTSVTVSS |
| 8_HC_ | ASTKGPSVFPLAPSSKSTSGGTAALGCLVKDYFPEPVTVSWNSGALTSGVHTFPAVLQSSGL |
| hole | YSLSSVVTVPSSSLGTQTYICNVNHKPSNTKVDKRVEPKSCDKTHTCPPCPAPELLGGPSVFL |
| AA | FPPKPKDTLMISRTPEVTCVVVDVSHEDPEVKFNWYVDGVEVHNAKTKPREEQYASTYRV |
| | VSVLTVLHQDWLNGKEYKCKVSNKALPAPIEKTISKAKGQPREPQVYTLPPSREEMTKNQV |
| | SLTCLVKGFYPSDIAVEWESNGQPENNYKTTPPVLDSDGSFLLYSKLTVDKSRWQQGNVFS |
| | CSVMHEALHNHYTQKSLSLSPGK (SEQ ID NO: 188) |
| SD- | GAAGTGAAGCTTGATGAGTCTGGGGGAGGCTTAGTGAAGCCTGGAGGGTCCCTGAAA |
| 17407 | CTCTCCTGTGCAGCCTCTGGATTCACTTTCAGTAGCTATACCATGTCTTGGGTTCGCCAG |
| 8_HC_ | ACTCCGGAGAAGAGGCTGGAGTGGGTCGCAACTATTAATAGTGATGGTATTTACACCT |
| hole | ACTATCCAGACAGTGTGAAGGGCCGATTCACCATCTCCAGAGACAATGCCAAGAACAC |
| DNA | CCTGTACCTGCAAATGAGCAGTCTGAAGTCTGAGGACACAGCCATGTATTACTGTACAA |
| | GAGTACATTACTACGGCTACGATGCTATGGACTACTGGGGTCAAGGAACCTCAGTCAC |
| | CGTCTCCTCAGCCAGCACCAAGGGCCccagcgtgttccccctggcccccagcagcaagagcaccagcg |
| | gcggcaccgccgccctgggctgcctggtgaaggactacttccccgagcccgtgaccgtgagctggaacagcggcg |
| | ccctgaccagcggcgtgcacaccttccccgccgtgctgcagagcagcggcctgtacagcctgagcagcgtggtga |
| | ccgtgcccagcagcagcctgggcacccagacctacatctgcaacgtgaaccacaagcccagcaacaccaaggtg |
| | gacaagagagtggagcccaagagctgcgacaagacccacacctgccccccctgccccgcccccgagctgctggg |
| | cggccccagcgtgttcctgttcccccccaagcccaaggacaccctgatgatcagcagaacccccgaggtgacctgc |
| | gtggtggtggacgtgagccacgaggaccccgaggtgaagttcaactggtacgtggacggcgtggaggtgcacaa |
| | cgccaagaccaagcccagagaggagcagtacgcgagcacctacagagtggtgagcgtgctgaccgtgctgcacc |
| | aggactggctgaacggcaaggagtacaagtgcaaggtgagcaacaaggccctgcccgcccccatcgagaagac |
| | catcagcaaggccaagggccagcccagagagccccaggtgtacaccctgccccccagcagagaggagatgacc |
| | aagaaccaggtgagcctgacctgcctggtgaagggcttctaccccagcgacatcgccgtggagtgggagagcaac |
| | ggccagcccgagaacaactacaagaccaccccccccgtgctggacagcgacggcagcttcctgctgtacagcaa |
| | gctgaccgtggacaagagcagatggcagcagggcaacgtgttcagctgcagcgtgatgcacgaggccctgcaca |
| | accactacacccagaagagcctgagcctgagccccggcaag (SEQ ID NO: 189) |
| SD- | EVQLVESGGGLVQPGGSLRLSCAASGFDFNAYAMNWVRQAPGKGLEWVGRIRSKYNNY |
| 23183 | ATYYADSVKGRFTISRDDSKNTLYLQMNSLRAEDTAVYYCVRHGNFGNSYVSWFAYWGQ |
| 1_HC_ | GTLVTVSSASTKGPSVFPLAPSSKSTSGGTAALGCLVKDYFPEPVTVSWNSGALTSGVHTFP |
| knob | AVLQSSGLYSLSSVVTVPSSSLGTQTYICNVNHKPSNTKVDKRVEPKSCDKTHTCPPCPAPEL |
| AA | LGGPSVFLFPPKPKDTLMISRTPEVTCVVVDVSHEDPEVKFNWYVDGVEVHNAKTKPREE |
| | QYASTYRVVSVLTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLYSRLTVDKSRW |
| | QQGNVFSCSVMHEALHNHYTQKSLSLSPGK (SEQ ID NO: 190) |
| SD- | GAGGTCCAACTCGTGGAATCAGGCGGGGGATTAGTGCAGCCTGGCGGTTCCCTTAGG |
| 23183 | CTCTCGTGTGCTGCATCTGGCTTCGACTTTAATGCCTACGCCATGAACTGGGTGCGTCA |
| 1_HC_ | GGCACCCGGAAAGGGATTGGAGTGGGTCGGACGCATTCGCAGCAAGTACAATAACTA |
| knob | CGCCACTTACTACGCTGATTCCGTGAAAGGCCGATTCACAATCAGCCGGGACGATAGC |
| DNA | AAAAACACCCTGTATCTGCAGATGAATAGTCTGAGGGCCGAAGACACAGCCGTGTACT |
| | ATTGCGTTCGGCACGGGAACTTTGGCAATTCTTATGTATCCTGGTTCGCCTACTGGGGG |
| | CAGGGCACTCTGGTCACCGTTTCTAGTGCGTCAACAAAAGGTCCCTCAGTGTTTCCTCT |
| | GGCCCCATCAAGCAAGAGCACCTCCGGCGGCACCGCCGCACTGGGATGTCTCGTGAAG |
| | GATTACTTCCCTGAGCCTGTAACTGTGAGCTGGAATTCTGGTGCGCTGACAAGTGGTGT |
| | CCATACCTTCCCCGCCGTCTTGCAGAGCAGTGGGCTGTATAGCTTAAGCAGCGTAGTGA |
| | CCGTCCCTAGCTCCTCCCTGGGTACTCAGACTTATATCTGCAATGTCAATCATAAACCCT |
| | CTAATACCAAAGTCGATAAACGCGTGGAGCCTAAATCTTGTGATAAAACCCATACCTGC |
| | CCACCATGTCCCGCCCCTGAGCTGCTCGGCGGTCCATCCGTCTTCCTGTTTCCTCCAAAA |
| | CCAAAGGACACATTGATGATTAGTAGGACACCCGAGGTCACCTGTGTGGTGGTTGACG |
| | TTTCCCACGAGGACCCAGAGGTCAAATTCAACTGGTATGTGGACGGCGTGGAGGTCCA |
| | CAACGCTAAAACCAAACCCAGAGAAGAACAGTACGCCTCTACTTATAGAGTTGTGAGC |
| | GTCCTCACGGTGCTGCACCAGGATTGGCTGAATGGCAAGGAGTATAAGTGCAAAGTCT |
| | CTAATAAAGCACTCCCTGCGCCAATAGAGAAGACAATTAGCAAGGCAAAGGGACAACC |
| | ACGCGAACCTCAGGTGTACACTCTCCCCCCTAGCCGGGAAGAGATGACAAAAAATCAG |
| | GTTTCTTTAACATGTCTGGTGAAAGGATTTTACCCTTCCGACATCGCGGTCGAATGGGA |
| | GTCAAATGGACAACCCGAGAATAATTACAAGACAACACCACCAGTGTTAGACTCCGAT |
| | GGCTCCTTCTTTCTGTACAGCAGGCTGACGGTGGACAAGAGCCGCTGGCAACAGGGGA |
| | ATGTCTTCTCTTGCTCTGTGATGCATGAGGCACTCCACAATCATTATACGCAGAAAAGTC |
| | TCTCTCTCTCTCCAGGCAAA (SEQ ID NO: 191) |
| SD- | QVQLLESGGGLVKPGGSLRLSCAASGFDFIDYDMFWIRQAPGKGLEWVGVINRSGSYTNY |
| 23183 | APAVKGRATISRDNAKNSMYLQMNSLRAEDTAVYYCARGACTHCWDADSAGYIDAWGQ |
| 1_HC_ | GTLVTVSSASTKGPSVFPLAPSSKSTSGGTAALGCLVKDYFPEPVTVSWNSGALTSGVHTFP |
| hole | AVLQSSGLYSLSSVVTVPSSSLGTQTYICNVNHKPSNTKVDKRVEPKSCDKTHTCPPCPAPEL |
| AA | LGGPSVFLFPPKPKDTLMISRTPEVTCVVVDVSHEDPEVKFNWYVDGVEVHNAKTKPREE |
| | QYASTYRVVSVLTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFLLYSKLTVDKSRW |
| | QQGNVFSCSVMHEALHNHYTQKSLSLSPGK (SEQ ID NO: 192) |
| SD- | CAAGTACAGTTGCTGGAGTCAGGGGGTGGGTTAGTGAAGCCAGGAGGCAGCCTTCGC |
| 23183 | TTGTCCTGCGCAGCCTCTGGTTTTGATTTCATCGACTACGATATGTTCTGGATCCGGCAG |
| 1_HC_ | GCCCCTGGGAAAGGGCTGGAGTGGGTAGGCGTCATCAACAGGTCCGGTTCTTATACCA |
| hole | ATTACGCCCCTGCCGTAAAGGGAAGAGCTACGATCAGTCGCGACAACGCGAAAAACTC |
| DNA | TATGTATCTCCAAATGAATTCACTGCGGGCAGAAGACACAGCCGTGTATTACTGTGCGC |
| | GGGGCGCGTGTACGCATTGCTGGGATGCAGACTCCGCCGGATACATCGATGCTTGGG |
| | GGCAGGGAACTTTGGTAACCGTGAGCTCCGCATCCACCAAGGGCCCcagcgtgttccccctg |
| | gcccccagcagcaagagcaccagcggcggcaccgccgccctgggctgcctggtgaaggactacttccccgagccc |
| | gtgaccgtgagctggaacagcggcgccctgaccagcggcgtgcacaccttccccgccgtgctgcagagcagcggc |
| | ctgtacagcctgagcagcgtggtgaccgtgcccagcagcagcctgggcacccagacctacatctgcaacgtgaac |
| | cacaagcccagcaacaccaaggtggacaagagagtggagcccaagagctgcgacaagacccacacctgccccc |
| | cctgccccgcccccgagctgctgggcggccccagcgtgttcctgttcccccccaagcccaaggacaccctgatgat |
| | cagcagaacccccgaggtgacctgcgtggtggtggacgtgagccacgaggaccccgaggtgaagttcaactggt |
| | acgtggacggcgtggaggtgcacaacgccaagaccaagcccagagaggagcagtacgcgagcacctacagagt |
| | ggtgagcgtgctgaccgtgctgcaccaggactggctgaacggcaaggagtacaagtgcaaggtgagcaacaagg |
| | ccctgcccgcccccatcgagaagaccatcagcaaggccaagggccagcccagagagccccaggtgtacaccctg |
| | ccccccagcagagaggagatgaccaagaaccaggtgagcctgacctgcctggtgaagggcttctaccccagcga |
| | catcgccgtggagtgggagagcaacggccagcccgagaacaactacaagaccaccccccccgtgctggacagcg |
| | acggcagcttcctgctgtacagcaagctgaccgtggacaagagcagatggcagcagggcaacgtgttcagctgca |
| | gcgtgatgcacgaggccctgcacaaccactacacccagaagagcctgagcctgagccccggcaag (SEQ ID |
| | NO: 193) |

Antibody humanization. Humanization of SD-589775 was accomplished via grafting of both heavy and light chain CDRs to closely related human germ line sequences, resulting in 589-VH_1 and 589-VL_1. Identified Vernier residues were manually reverted to the parental chicken amino acids, resulting in 589-VH_2 and 589-VL_2. An additional several amino acids in the VH were reverted to the parental chicken sequence to produced 589-VH_3. A publicly available tool (DOI: 10.1080/19420862.2021.2020203) was used to humanize the VH and VL, resulting in 589-VH_4 and 589-VL_3. Parental chicken sequences were used as 589-VH_5 and 589-VL_4. The combination of 589-VH_2 and 589-VL_2 yields SD-861408. Testing was done via SPR as previously described.

CD3ϵδ binding by direct ELISA. Antibody binding to recombinant CD3ϵδ-HRP heterodimer (Acro Biosystems CDD-HR2W3) was assessed by a direct ELISA. Each antibody was diluted to 5 μg/mL in 50 μL/well of 50 mM carbonate buffer (pH 9.5) and plated for 1 hour. Following the coating step, plates were blocked for 15 minutes with 300 μL/well of SuperBlock™ blocking buffer (Thermo Fisher Scientific #37515) at room temperature. A 7-point dose-response curve for the recombinant CD3ϵδ-HRP conjugate was then generated by 5-fold serial dilution beginning at 10 μg/mL in PBS and incubated on the plate for 1 hour. Plates were developed using 1-Step™ TMB ELISA substrate solution (Thermo Fisher Scientific #34028) for 5 minutes and stopped with a 1:1 mixture of stop solution (Thermo Fisher Scientific #SS04). Absorbance of each well at 450 nm was plotted against the log of the concentration of CD3ϵδ-HRP and fit with non-linear regression to calculate EC50. Each incubation step was carried out at room temperature for 1 hour with light agitation with PBST washes in between.

Assembly of bispecific TROP2×CD3 antibodies. Bispecific knob-in-hole antibodies were generated using controlled Fab-arm exchange (DOI: 10.1038/nprot.2014.169). Monospecific parent kno https://doi.org/10.1038/nprot.2014.169 b (K409R; CD3 binding) and hole (F405L; TROP2 or antigen binding) antibodies were mixed at a 1:1 ratio in PBS with 75mM 2-mercaptoethylamine hydrochloride (2-MEA, Millipore Sigma #M6500) and incubated for 5 hours at 31° C. After incubation, 2-MEA was removed by buffer exchange into PBS using a Zeba™ spin desalting column (Thermo Fisher Scientific #87767), filtered using a 0.2 μm filter, and incubated overnight at 4° C. before testing assembly or activity. All bispecific antibodies had Fc effector function silenced via the N297A mutation.

Bispecific antibody assembly efficiency by cation-exchange HPLC. Assembly efficiency of bispecific antibodies was assessed with HPLC (Agilent Infinity 1290) using a ProPac WCX-10 25 cm cation-exchange column (Thermo Fisher Scientific #054993). If necessary, the formulation buffer of the antibody to measure was exchanged to Buffer A (20 mM NaPi, pH 7.0) using a Zeba™ spin desalting column (Thermo Fisher Scientific #87767) before filtering using a 0.2 μm filter. After equilibrating the column with Buffer A, samples were loaded onto a 96-well plate and placed into the instrument. 20 μL of each sample was then injected over the column with the following gradient conditions: 0-3 min, 100% Buffer A; 3-58.5 min, 0-72% Buffer B (20 mM NaPi, pH 7.0+250 mM NaCl); 58-5-63.5 min, 100% Buffer C (20 mM NaPi, pH 7.0+750 mM NaCl); 63.5-82 min, 100% Buffer A. The resulting peaks were integrated using OpenLab Chemstation (Agilent Technologies). Percent assembly was calculated by dividing the area corresponding to the bispecific antibody peak by the total peak area.

T-cell dependent cellular cytotoxicity (TDCC) and T cell activation assay. Ovcar3 cells (ATCC, HTB-161) were cultured in RPMI (ATCC, 30-2001) supplemented with 20% FBS (Sigma, F5135) and 1× Penicillin-Streptomycin (Corning, 30-002-CI), 0.01 mg/ml bovine insulin. FADU cells (ATCC, HTB-43) were cultured in EMEM (ATCC, 30-2003) supplemented with 10% FBS (Sigma, F5135) and 1× Penicillin-Streptomycin (Corning, 30-002-CI). Calu-3 cells (ATCC, HTB-55) were cultured in EMEM (ATCC, 30-2003) supplemented with 10% FBS (Sigma, F5135) and 1× Penicillin-Streptomycin (Corning, 30-002-CI).

For TDCC PBMC assay, Ovcar3 or FADU or Calu-3 cells were counted to assess the cell number and viability. The cells were stained with CFSC. Cells were centrifuged and resuspended in growth media at 1×10\hat{ }5 cells/ml seeded at 1×10\hat{ }4 cells per well onto 96 well plates (VWR, 89131-676) and incubated in cell culture incubator overnight. Anti-TROP2 or control antibodies in Assay media were added to the cells at increasing concentrations (0.032-500 ng/ml) for 20 minutes at 37° C., 5% CO₂. After that, 2×10\hat{ }5 peripheral blood mononuclear cells (PBMC) (Stemcell, 70025.1) were added per well of 96 well plate. Cells and antibodies were incubated at 37° C. in 5% CO₂ incubator for 48 hours. Samples were then stained with LIVE/DEAD™ fixable Aqua Dead Cell Stain Kit (Thermo Fisher, L34957), and antibodies cocktail (CD32/CD16 blocker, CD3-BV605, CD4-PerCP/Cy5.5, CD8a-APC, CD69-PE, CD25+-APC/cy7 (Biolegend)). Samples then analyze using flow cytometer (MacsQuant 16, Miltenyi). The % dead target cell was gated for FITC+ Live/dead +. The early activated CD4+ was gated as: Live/dead−/CD3+/CD4+/CD69+. The early activated CD8+ was gated as: Live/dead−/CD3+/CD8+/CD69+. The later activated CD4+ was gated as: Live/dead−/CD3+CD4+/CD25+. The late activated CD8+ was gated as: Live/dead−/CD3+/CD8+/CD69+. The EC50 value was calculated using Graphpad Prism 9.3.0.

Measurement of cytokine release. Cytokine release was measured using a custom U-PLEX Biomarker Assay kit (Meso Scale Discovery K15067M) allowing for quantification of IFNγ, IL-2, IL-6, and TNFα according to the kit instructions. Briefly, biotinylated capture antibodies were conjugated to unique linkers and applied to the plate to capture them on particular spots. Cell-culture supernatants were taken from the TDCC assay at the 24-hour time point and diluted 1:10 in Diluent 57 before being applied to the plate, after which a mixture of detection antibodies diluted in Diluent 3 was added to each well. Each incubation step was carried out for 1 hour at room temperature with light agitation with 3×150 μL washes with PBST in between. After the final wash, 150 μL/well of MSD GOLD Read Buffer B was added and the plate was read on the MESO QuickPlex SQ 120MM instrument. Cytokine concentrations were determined using the Discovery Workbench software.

Provided herein are sequences for exemplary anti-Trop 2 and bi-specific antibodies of the disclosure. Included are complementarity determining region (CDR) sequences and the variable heavy and light domain sequences (VH, VL) that constitute the anti-Trop 2 antigen binding domains of the disclosure. The discovery of these antibodies is detailed in the Examples section.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3.Table 1 provides exemplary CDR combinations of antibodies of the disclosure. scFv-Fc TROP 2 Antibodies.

In some embodiments, the disclosure provides for tandem scFv antibodies, with multiple Trop 2 binding sites. Tandem scFv-Fc antibodies of the disclosure are composed two or more scFv binding sites in tandem on each antibody arm, optionally linked by a linker, optionally a flexible linker. In some embodiments, a tandem scFV antibody has a total of four or more scFv binding sites in a single scFv-Fc formatted antibody.

In some embodiments, the scFv1 of each antibody arm comprises a first heavy chain variable domain (VH1) and a first light chain variable domain (VL1); and the scFv2 of each antibody arm comprises a first heavy chain variable domain (VH2) and a first light chain variable domain (VL2).

Therapeutic anti-Trop 2 and bi-specific antibodies.

In some embodiments, the anti-Trop 2 and bi-specific antibodies (TROP2×CD3) provided herein are useful for the treatment of a disease or condition involving an immune response.

In some embodiments, the anti-Trop 2 and bi-specific antibodies provided herein are useful for the treatment of a proliferative diseases.

The in vivo administration of the therapeutic anti-Trop 2 and bi-specific antibodies

described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the anti-Trop 2 and bi-specific antibodies of the disclosure are administered intravenously. In some embodiments, anti-Trop 2 and bi-specific antibodies of the disclosure are administered subcutaneously. Administration of the therapeutic anti-Trop 2 and bi-specific antibodies may be performed with any suitable excipients, carriers, or other agents to provide suitable or improved tolerance, transfer, delivery, and the like.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An anti-Trop 2 antibody or binding fragment thereof, wherein the antibody comprises:
a. a heavy chain variable domain (VH) complementarity determining region (CDR) 1 comprising the amino acid sequence of any one of the following SEQ ID NOs: 2, 12, 22, 32, 42, 52, 62, 72, 82, 92, 102, 112, 122, or 132; and
b. a VH CDR2 comprising the amino acid sequence of any one of the following SEQ ID NOs: 3, 13, 23, 33, 43, 53, 63, 73, 83, 93, 103, 113, 123, or 133; and
c. a VH CDR3 comprising the amino acid sequence of any one of the following SEQ ID NOs: 4, 14, 24, 34, 44, 54, 64, 74, 84, 94, 104, 114, 124, 134; and
d. a light chain variable domain (VL) CDR 1 comprising the amino acid sequence of any one of the following SEQ ID NOs: 7, 17, 27, 37, 47, 57, 67, 77, 87, 97, 107, 117, 127, or 137; and
e. a VL CDR2 comprising the amino acid sequence of any one of the following SEQ ID NOs: 8, 18, 28, 38, 48, 58, 68, 78, 88, 98, 108, 118, 128, or 138; and
f. a VL CDR3 comprising the amino acid sequence of any one of the following SEQ ID NOs: 9, 19, 29, 39, 49, 59, 69, 79, 89, 99, 109, 119, 129, or 139.

2. The antibody or binding fragment of claim 1, wherein the antibody comprises:
a. the VH comprising the amino acid sequence of any one of the following SEQ ID NOs: 1, 11, 21, 31, 41, 51, 61, 71, 81, 91, 101, 111, 121, 131, 156, 158, 160, or 162, and
b. the VL comprising the amino acid sequence of any one of the following SEQ ID NOs: 6, 16, 26, 36, 46, 56, 66, 76, 86, 96, 106, 116, 126, 136, 164, 166, or 168.

3. The antibody or binding fragment of claim 1, wherein the antibody comprises:
a. the VH is encoded by a nucleic acid sequence comprising of any one of the following SEQ ID NOs: 5, 15, 25, 35, 45, 55, 65, 75, 85, 95, 105, 115, 125, 135, 157, 159, 161, or 163, and
b. the VL is encoded by a nucleic acid sequence comprising of any one of the following SEQ ID NOs: 10, 20, 30, 40, 50, 60, 70, 80, 90, 100, 110, 120, 130, 140, 165, 167,or 169.

4. The antibody or binding fragment of claim 1, wherein the antibody is at least one of:
a monoclonal, bispecific, multivalent, multi-specific, diabody, chimeric, scFv antibody, or fragments thereof;
a full-length antibody that is afucosylated;
an Fc domain of any one of the following: human IgG1, human IgG2, human IgG3, and human IgG4; or
the Fc domain is a wild-type, variant, or truncated Fc domain.

5. The antibody or binding fragment of claim 1, further comprising a second antigen binding domain that binds to a target other than TROP-2.

6. The antibody or binding fragment of claim 5, wherein the target is CD3 antibody with a heavy chain selected from SEQ ID NO: 174 or 178; and the light chain is selected from SEQ ID NOS: 176 or 180, or a bispecific antibody that comprises SEQ ID NO:174, SEQ ID NO: 126, SEQ ID NO: 190, and SEQ ID NO:166; or wherein the antibody is a bispecific antibody comprising a heavy chain selected from SEQ ID NOS: 190 and 192, or 194 and 196.

7. A method of treating a disease in a subject in need thereof, comprising administering to the subject a therapeutically effective amount of the antibody of claim 1.

8. The method of claim 7, wherein the disease is a proliferative disease.

9. The method of claim 8, wherein the proliferative disease is cancer selected from those that express Trop 2 or a mutant thereof.

10. The method of claim 9, wherein the subject is human.

11. A bi-valent or multi-valent antibody wherein said antibody comprises:
an anti-Trop 2 antibody or binding fragment thereof, wherein the antibody comprises:
a. a heavy chain variable domain (VH) complementarity determining region (CDR) 1 comprising the amino acid sequence of any one of the following SEQ ID NOs: 2, 12, 22, 32, 42, 52, 62, 72, 82, 92, 102, 112, 122, or 132; and
b. a VH CDR2 comprising the amino acid sequence of any one of the following SEQ ID NOs: 3, 13, 23, 33, 43, 53, 63, 73, 83, 93, 103, 113, 123, or 133; and
c. a VH CDR3 comprising the amino acid sequence of any one of the following SEQ ID NOs: 4, 14, 24, 34, 44, 54, 64, 74, 84, 94, 104, 114, 124, 134; and
d. a light chain variable domain (VL) CDR1 comprising the amino acid sequence of any one of the following SEQ ID NOs: 7, 17, 27, 37, 47, 57, 67, 77, 87, 97, 107, 117, 127, or 137; and
e. a VL CDR2 comprising the amino acid sequence of any one of the following SEQ ID NOs: 8, 18, 28, 38, 48, 58, 68, 78, 88, 98, 108, 118, 128, or 138; and
f. a VL CDR3 comprising the amino acid sequence of any one of the following SEQ ID NOs: 9, 19, 29, 39, 49, 59, 69, 79, 89, 99, 109, 119, 129, or 139; and
a second antibody, antigen-binding of the second antibody or fragment thereof; a target-binding protein, a cytokine; a lectin; or a toxin.

12. The bi-valent or multi-valent antibody of claim 11, wherein the second antibody targets an immune effector cell surface receptor selected from at least one of CTLA-4, PD-1, Lag3, S15, B7H3, B7H4, TCR-alpha, TCR-beta, TIM-3, CD3, 41BB or OX40.

13. The bi-valent or multi-valent antibody of claim 12, wherein the antibody is a multi-valent antibody that targets two or more antigens other than Trop 2.

14. The bi-valent or multi-valent antibody of claim 11, wherein the antibody is a bispecific antibody comprising a heavy chain selected from SEQ ID NOS: 190 and 192, 194 and 196, or a bispecific antibody that comprises SEQ ID NO:174, SEQ ID NO:126, SEQ ID NO: 190, and SEQ ID NO:166.

15. The bi-valent or multi-valent antibody of claim 11, wherein the anti-Trop 2 antibody comprises:
a. the VH comprising the amino acid sequence of any one of the following SEQ ID NOs: 1, 11, 21, 31, 41, 51, 61, 71, 81, 91, 101, 111, 121, 131, 156, 158, 160, or 162; and
b. the VL comprising the amino acid sequence of any one of the following SEQ ID NOs: 6, 16, 26, 36, 46, 56, 66, 76, 86, 96, 106, 116, 126, 136, 164, 166, or 168.

16. The bi-valent or multi-valent antibody of claim 11, wherein the anti-Trop 2 antibody comprises:
a. the VH is encoded by a nucleic acid sequence comprising of any one of the following SEQ ID NOs: 5, 15, 25, 35, 45, 55, 65, 75, 85, 95, 105, 115, 125, 135, 157, 159, 161, or 163, and
b. the VL is encoded by a nucleic acid sequence comprising of any one of the following SEQ ID NOs: 10, 20, 30, 40, 50, 60, 70, 80, 90, 100, 110, 120, 130, 140, 165, 167, or 169.

17. The bi-valent or multi-valent antibody of claim 15, wherein the antibody is at least one of:
a monoclonal antibody, a full-length antibody, or an antibody fragment; or
the antibody is fused to an Fc domain of any one of the following: human IgG1, human IgG2, human IgG3, and human IgG4; or
the Fc domain is a wild-type, variant, or truncated Fc domain; or the antibody is a full-length antibody that is afucosylated.

18. The bi-valent or multi-valent antibody of claim 15, wherein the target is CD3 and the antibody a heavy chain selected from SEQ ID NO: 174 or 178; and the light chain is selected from SEQ ID NOS: 176 or 180; or the target is CD3 and the antibody a heavy chain is encoded by a nucleic acid selected from SEQ ID NO: 175 or 179; and the light chain is encoded by a nucleic acid selected from SEQ ID NOS: 177 or 181.

19. A method of treating a disease in a subject in need thereof, comprising administering to the subject a therapeutically effective amount of an anti-Trop 2 antibody or binding fragment thereof, wherein the antibody comprises:
a. a heavy chain variable domain (VH) complementarity determining region (CDR) 1 comprising the amino acid sequence of any one of the following SEQ ID NOs: 2, 12, 22, 32, 42, 52, 62, 72, 82, 92, 102, 112, 122, or 132; and
b. a VH CDR2 comprising the amino acid sequence of any one of the following SEQ ID NOs: 3, 13, 23, 33, 43, 53, 63, 73, 83, 93, 103, 113, 123, or 133; and
c. a VH CDR3 comprising the amino acid sequence of any one of the following SEQ ID NOs: 4, 14, 24, 34, 44, 54, 64, 74, 84, 94, 104, 114, 124, 134; and
d. a light chain variable domain (VL) CDR1 comprising the amino acid sequence of any one of the following SEQ ID NOs: 7, 17, 27, 37, 47, 57, 67, 77, 87, 97, 107, 117, 127, or 137; and
e. a VL CDR2 comprising the amino acid sequence of any one of the following SEQ ID NOs: 8, 18, 28, 38, 48, 58, 68, 78, 88, 98, 108, 118, 128, or 138; and
a VL CDR3 comprising the amino acid sequence of any one of the following SEQ ID NOs: 9, 19, 29, 39, 49, 59, 69, 79, 89, 99, 109, 119, 129, or 139.

20. The method of claim 19, wherein the disease is a proliferative disorder selected from a cancer cell that expresses Trop 2 or mutants thereof selected from cancers of the breast, cervix, colorectal, esophagus, gastric, certain lung cancers, squamous cell carcinoma of the oral cavity, ovary, pancreas, prostate, stomach, thyroid, urinary bladder, and uterus.

21. The method of claim 19, wherein the subject is human.

22. A nucleic acid encoding an antibody, binding fragment thereof, bivalent, or multivalent nucleic acid sequence comprising:
a. a VH is encoded by a nucleic acid sequence comprising of any one of the following SEQ ID NOs: 5, 15, 25, 35, 45, 55, 65, 75, 85, 95, 105, 115, 125, 135, 157, 159, 161, or 163, and
b. a VL is encoded by a nucleic acid sequence comprising of any one of the following SEQ ID NOs: 10, 20, 30, 40, 50, 60, 70, 80, 90, 100, 110, 120, 130, 140, 165, 167, or 169; or
c. an antibody encoded by a nucleic acid sequence of SEQ ID NOS: 187 and 189, or 191 and 193; or
d. a bispecific antibody encoded by a nucleic acid sequence of SEQ ID NOS: 175 and 177, 179 and 181, 183 and 185, 187 and 189, or 191 and 193, or combinations thereof.

23. The nucleic acid of claim 22, further comprising a vector.

24. The nucleic acid of claim 23, wherein the vectors is in a host cell.


SEQUENCE LISTING (193 sequences)

SEQ ID NO: 1 - protein, 115 aa, synthetic construct
  REGION 1..115: note = Synthetic: SD-025096_VH
QVQLQQSGAELVRPGASVKLSCKASGYTFTSYWINWVKQRPGQGLEWIGNIFPSGSYTNYNQKFKDRATLTVDKSSSTAFMQLSSPTSEDSAIYFCTRGSGFDYWGQGTTLTVSS

SEQ ID NO: 2 - protein, 5 aa, synthetic construct
  REGION 1..5: note = Synthetic: SD-025096_VH_CDR1
SYWIN

SEQ ID NO: 3 - protein, 17 aa, synthetic construct
  REGION 1..17: note = Synthetic: SD-025096_VH_CDR2
NIFPSGSYTNYNQKFKD

SEQ ID NO: 4 - protein, 6 aa, synthetic construct
  REGION 1..6: note = Synthetic: SD-025096_VH_CDR3
GSGFDY

SEQ ID NO: 5 - DNA, 345 bp, synthetic construct
  misc_feature 1..345: note = Synthetic: SD-025096_VH_DNA
  source 1..345: mol_type = other DNA
caggttcagctgcagcagtctggggctgagctggtgaggcctggggcttcagtgaagctgtcctgcaaggcttctggctacaccttcaccagctactggataaactgggtgaagcagaggcctggacaaggccttgagtggatcggaaatatttttccttctggtagttatactaactacaatcaaaagttcaaggacagggccacattgactgtagacaaatcctccagcacagccttcatgcaactcagcagcccgacatctgaggactctgcgatctatttctgtacaaggggtagtggcttcgactactggggccaaggcaccactctcacggtctcctca

SEQ ID NO: 6 - protein, 107 aa, synthetic construct
  REGION 1..107: note = Synthetic: SD-025096_VL
DIVLTQSPATLSVTPGDSVSLSCRASQSISNNLHWYQQKSHESPRLLIKYASQSISGIPSRFSGSGSGTDFTLSINSVETEDFGMYFCQQSNSWPFTFGSGTKLEIK

SEQ ID NO: 7 - protein, 11 aa, synthetic construct
  REGION 1..11: note = Synthetic: SD-025096_VL_CDR1
RASQSISNNLH

SEQ ID NO: 8 - protein, 7 aa, synthetic construct
  REGION 1..7: note = Synthetic: SD-025096_VL_CDR2
YASQSIS

SEQ ID NO: 9 - protein, 9 aa, synthetic construct
  REGION 1..9: note = Synthetic: SD-025096_VL_CDR3
QQSNSWPFT

SEQ ID NO: 10 - DNA, 321 bp, synthetic construct
  misc_feature 1..321: note = Synthetic: SD-025096_VL_DNA
  source 1..321: mol_type = other DNA
gatattgtgctaacccagtctccagccaccctgtctgtgactccaggagatagcgtcagtctttcctgcagggccagccaaagtattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggccattcacgttcggctcggggacaaagttggaaataaaa

SEQ ID NO: 11 - protein, 125 aa, synthetic construct
  REGION 1..125: note = Synthetic: SD-071877_VH
AVTLDESGGGLQTPGGALSLVCKASGFDFSGYGMGWVRQAPGKGLEWVAGITNDGRYIGHGSAVKGRATISRDNGQSTVRLQLNNLRAEDTGTYFCTKCAYRSGCDYEAGSIDAWGHGTEVIVSS

SEQ ID NO: 12 - protein, 5 aa, synthetic construct
  REGION 1..5: note = Synthetic: SD-071877_VH_CDR1
GYGMG

SEQ ID NO: 13 - protein, 17 aa, synthetic construct
  REGION 1..17: note = Synthetic: SD-071877_VH_CDR2
GITNDGRYIGHGSAVKG

SEQ ID NO: 14 - protein, 16 aa, synthetic construct
  REGION 1..16: note = Synthetic: SD-071877_VH_CDR3
CAYRSGCDYEAGSIDA

SEQ ID NO: 15 - DNA, 375 bp, synthetic construct
  misc_feature 1..375: note = Synthetic: SD-071877_VH_DNA
  source 1..375: mol_type = other DNA
gccgtgacgttggacgagtccgggggcggcctccagacgcccggaggagcgctcagcctcgtctgcaaggcctccgggtttgacttcagcggttatggcatgggttgggtgcgccaggcgcccggcaaagggctggaatgggtcgctggtattaccaatgatggtagatatataggccacgggtcggcggtgaagggccgtgccaccatctcgagggacaacgggcagagcacagtgaggctgcagctgaacaacctcagggctgaggacaccggcacctacttctgcacgaaatgtgcttatcgtagtggttgtgattatgaagctggttctatcgacgcatggggccacgggaccgaagtcatcgtctcctcc

SEQ ID NO: 16 - protein, 107 aa, synthetic construct
  REGION 1..107: note = Synthetic: SD-071877_VL
ALALTQPSSVSANLGGTVKITCSGDNNWYGWYQQKSPGSAPVTVIYYDDKRPSDIPSRFSGSRSGSTGTLTITGVQAEDEAVYFCGNRDSSAGYVGIFGAGTTLTVL

SEQ ID NO: 17 - protein, 8 aa, synthetic construct
  REGION 1..8: note = Synthetic: SD-071877_VL_CDR1
SGDNNWYG

SEQ ID NO: 18 - protein, 7 aa, synthetic construct
  REGION 1..7: note = Synthetic: SD-071877_VL_CDR2
YDDKRPS

SEQ ID NO: 19 - protein, 12 aa, synthetic construct
  REGION 1..12: note = Synthetic: SD-071877_VL_CDR3
GNRDSSAGYVGI

SEQ ID NO: 20 - DNA, 321 bp, synthetic construct
  misc_feature 1..321: note = Synthetic: SD-071877_VL_DNA
  source 1..321: mol_type = other DNA
gcacttgcgctgactcagccgtcctcggtgtcagcaaacctgggaggaaccgtcaagatcacctgctccggggataacaattggtatggctggtatcagcagaagtcacctggcagtgcccctgtcactgtgatctattacgacgacaagagaccctcggacatcccttcacgattctccggttccagatccggctccacgggcacattaaccatcactggggtccaagccgaggacgaggctgtctatttctgtggaaacagggacagcagtgctggttatgttggtatatttggggccgggacaaccctgaccgtccta

SEQ ID NO: 21 - protein, 122 aa, synthetic construct
  REGION 1..122: note = Synthetic: SD-168804_VH
AVTLDESGGGLQTPGGALSLVCKASGFTFSDRGMGWVRQVPGKGLEWVAAISSTVTYTSYGPAVKGRATISRDDGQSTVRLQLNNLRAEDTGTYYCAKGPYRGIYGSEIDAWGHGTEVIVSS

SEQ ID NO: 22 - protein, 5 aa, synthetic construct
  REGION 1..5: note = Synthetic: SD-168804_VH_CDR1
DRGMG

SEQ ID NO: 23 - protein, 17 aa, synthetic construct
  REGION 1..17: note = Synthetic: SD-168804_VH_CDR2
AISSTVTYTSYGPAVKG

SEQ ID NO: 24 - protein, 13 aa, synthetic construct
  REGION 1..13: note = Synthetic: SD-168804_VH_CDR3
GPYRGIYGSEIDA

SEQ ID NO: 25 - DNA, 366 bp, synthetic construct
  misc_feature 1..366: note = Synthetic: SD-168804_VH_DNA
  source 1..366: mol_type = other DNA
gccgtgacgttggacgagtccgggggcggcctccagacgcccggaggagcgctcagcctcgtctgcaaggcctccgggttcaccttcagtgaccgtggcatgggttgggtgcgacaggtgcccggcaaggggctggagtgggtcgctgctattagcagcactgtcacttacacatcatatgggccggcggtgaagggccgtgccaccatctcgagggacgatgggcagagcacagtgaggctgcaactgaacaacctcagggctgaggacaccggcacctactactgcgccaagggtccttaccgtggtatttatggtagtgaaatcgacgcatggggccacgggaccgaagtcatcgtctcctcc

SEQ ID NO: 26 - protein, 110 aa, synthetic construct
  REGION 1..110: note = Synthetic: SD-168804_VL
ALALTQPSSVSANPGETVKITCSGGSYSYGSYYYGWYQQKSPGSAPVTVIYDNDKRPSGIPSRFSGSKSGSTGTLTITGVQAEDEAVYFCGSTDSSSTAAFGAGTTLTVL

SEQ ID NO: 27 - protein, 13 aa, synthetic construct
  REGION 1..13: note = Synthetic: SD-168804_VL_CDR1
SGGSYSYGSYYYG

SEQ ID NO: 28 - protein, 7 aa, synthetic construct
  REGION 1..7: note = Synthetic: SD-168804_VL_CDR2
DNDKRPS

SEQ ID NO: 29 - protein, 10 aa, synthetic construct
  REGION 1..10: note = Synthetic: SD-168804_VL_CDR3
GSTDSSSTAA

SEQ ID NO: 30 - DNA, 330 bp, synthetic construct
  misc_feature 1..330: note = Synthetic: SD-168804_VL_DNA
  source 1..330: mol_type = other DNA
gcacttgcgctgactcagccgtcctcggtgtcagcaaacccgggagaaaccgtcaagatcacctgctctgggggcagctatagctatggaagttactattatggctggtaccagcagaagtcacctggcagtgccccagtcactgtgatctatgacaacgacaagagaccctcgggcatcccttcacgattctccggttccaaatccggctccacgggcacattaaccatcactggggtccaagccgaggacgaggctgtctatttctgtgggagtacagacagcagcagtactgctgcatttggggccgggacaaccctgaccgtccta

SEQ ID NO: 31 - protein, 121 aa, synthetic construct
  REGION 1..121: note = Synthetic: SD-175533_VH
QIQLQESGPGLMKPSQSLSLTCTVTGYSITSDYAWNWIRQFPGNKLEWMGHITYSGSTSYNPSLKSRISISRDTSKNQFFLQLNSVTTGDTATYYCARGGSTLISGDFDYWGQGTTLTVSS

SEQ ID NO: 32 - protein, 6 aa, synthetic construct
  REGION 1..6: note = Synthetic: SD-175533_VH_CDR1
SDYAWN

SEQ ID NO: 33 - protein, 16 aa, synthetic construct
  REGION 1..16: note = Synthetic: SD-175533_VH_CDR2
HITYSGSTSYNPSLKS

SEQ ID NO: 34 - protein, 12 aa, synthetic construct
  REGION 1..12: note = Synthetic: SD-175533_VH_CDR3
GGSTLISGDFDY

SEQ ID NO: 35 - DNA, 363 bp, synthetic construct
  misc_feature 1..363: note = Synthetic: SD-175533_VH_DNA
  source 1..363: mol_type = other DNA
cagattcagcttcaggagtcaggacctggcctaatgaaaccttctcagtctctgtccctcacctgcactgtcactggctactcaatcaccagtgattatgcctggaactggatccggcagtttccaggaaacaaactggagtggatgggccacataacctacagtggtagcactagctacaacccatctctcaaaagtcgaatctctatctctcgagacacatccaagaaccagttcttcctgcagttgaattctgtgactactggggacacagccacatattactgtgcaagagggggatctactttgatttcaggggactttgactactggggccaaggcaccactctcacagtctcctca

SEQ ID NO: 36 - protein, 107 aa, synthetic construct
  REGION 1..107: note = Synthetic: SD-175533_VL
DIVLTQSHKFMSTSVGDRVSITCKASQDVSTAVAWYQQKPGQSPKLLIYSASYRYTGVPDRFTGSGSGTDFTFTISSVQAEDLAVYYCQQHYSTPLTFGAGTKLELK

SEQ ID NO: 37 - protein, 11 aa, synthetic construct
  REGION 1..11: note = Synthetic: SD-175533_VL_CDR1
KASQDVSTAVA

SEQ ID NO: 38 - protein, 7 aa, synthetic construct
  REGION 1..7: note = Synthetic: SD-175533_VL_CDR2
SASYRYT

SEQ ID NO: 39 - protein, 9 aa, synthetic construct
  REGION 1..9: note = Synthetic: SD-175533_VL_CDR3
QQHYSTPLT

SEQ ID NO: 40 - DNA, 321 bp, synthetic construct
  misc_feature 1..321: note = Synthetic: SD-175533_VL_DNA
  source 1..321: mol_type = other DNA
gacattgtgctgacccaatctcacaaattcatgtccacatcagtaggagacagggtcagcatcacctgcaaggccagtcaggatgtgagtactgctgtagcctggtatcaacagaaaccaggacaatctcctaaactactgatttactcggcatcctaccggtacactggagtccctgatcgcttcactggcagtggatctgggacggatttcactttcaccatcagcagtgtgcaggctgaagacctggcagtttattactgtcagcaacattatagtactcctctcacgttcggtgctgggaccaagctggagctgaaa

SEQ ID NO: 41 - protein, 126 aa, synthetic construct
  REGION 1..126: note = Synthetic: SD-320063_VH
AVTLDESGGGLQTPGGGLSLVCKASGFDFIDYDMFWVRQAPGKGLAWVGVINRSGSYTNYAPAVKGRATISRDNGQSTVRLQLNNLRAEDTGTYYCAKGVCTHCWDADSAGNIDAWGHGTEVIVSS

SEQ ID NO: 42 - protein, 5 aa, synthetic construct
  REGION 1..5: note = Synthetic: SD-320063_VH_CDR1
DYDMF

SEQ ID NO: 43 - protein, 17 aa, synthetic construct
  REGION 1..17: note = Synthetic: SD-320063_VH_CDR2
VINRSGSYTNYAPAVKG

SEQ ID NO: 44 - protein, 17 aa, synthetic construct
  REGION 1..17: note = Synthetic: SD-320063_VH_CDR3
GVCTHCWDADSAGNIDA

SEQ ID NO: 45 - DNA, 378 bp, synthetic construct
  misc_feature 1..378: note = Synthetic: SD-320063_VH_DNA
  source 1..378: mol_type = other DNA
gccgtgacgttggacgagtccgggggcggcctccagacgcctggaggagggctcagcctcgtctgcaaggcctccgggttcgacttcatcgattatgacatgttctgggtgcgacaggctccaggcaaggggctggcatgggtcggtgttattaacaggagtggtagttacacaaactacgcgccggcggtgaagggccgtgccaccatctcgagggacaacgggcagagcacagtgaggctgcagctgaacaacctcagggctgaggacaccggcacctactactgcgccaaaggtgtatgtactcattgttgggatgcagacagtgccggtaacatcgacgcatggggccacgggaccgaagtcatcgtctcctcc

SEQ ID NO: 46 - protein, 105 aa, synthetic construct
  REGION 1..105: note = Synthetic: SD-320063_VL
ALALTQPSSVSANPGETVEITCSGGSGNYGWFQQKSPGSAPVTVIYNNNYRLSDIPSRFSGSASGSTATLTITGVRVEDEAVYFCGSADSTYVGLFGAGTTLTVL

SEQ ID NO: 47 - protein, 8 aa, synthetic construct
  REGION 1..8: note = Synthetic: SD-320063_VL_CDR1
SGGSGNYG

SEQ ID NO: 48 - protein, 7 aa, synthetic construct
  REGION 1..7: note = Synthetic: SD-320063_VL_CDR2
NNNYRLS

SEQ ID NO: 49 - protein, 10 aa, synthetic construct
  REGION 1..10: note = Synthetic: SD-320063_VL_CDR3
GSADSTYVGL

SEQ ID NO: 50 - DNA, 315 bp, synthetic construct
  misc_feature 1..315: note = Synthetic: SD-320063_VL_DNA
  source 1..315: mol_type = other DNA
gcacttgcgctgactcagccgtcctcggtgtcagcgaacccgggagaaaccgtcgagatcacctgctccgggggtagtggcaactatggctggttccagcagaagtctcctggcagtgcccctgtcactgtgatctataacaacaactacagactctcggacatcccttcacgattctccggttccgcatctggctccacagccacattaaccatcactggggtccgagtcgaggacgaggctgtctatttctgtgggagtgcagacagcacttatgttggtttatttggggccgggacaaccctgaccgtccta

SEQ ID NO: 51 - protein, 123 aa, synthetic construct
  REGION 1..123: note = Synthetic: SD-394517_VH
AVTLDESGGGLQTPGGALSLVCKGSGFTFSSYAMGWVRQAPGKGLEYVAAISSGSSTGYGAAVKGRATISRDTGQSTVRLQLNNLRAEDTATYYCAKSGYGGSASYVSDIDAWGHGTEVIVSS

SEQ ID NO: 52 - protein, 5 aa, synthetic construct
  REGION 1..5: note = Synthetic: SD-394517_VH_CDR1
SYAMG

SEQ ID NO: 53 - protein, 16 aa, synthetic construct
  REGION 1..16: note = Synthetic: SD-394517_VH_CDR2
AISSGSSTGYGAAVKG

SEQ ID NO: 54 - protein, 15 aa, synthetic construct
  REGION 1..15: note = Synthetic: SD-394517_VH_CDR3
SGYGGSASYVSDIDA

SEQ ID NO: 55 - DNA, 369 bp, synthetic construct
  misc_feature 1..369: note = Synthetic: SD-394517_VH_DNA
  source 1..369: mol_type = other DNA
gccgtgacgttggacgagtccgggggcggcctccagacgcccggaggagcgctcagcctcgtctgcaagggctccgggttcaccttcagcagttatgccatgggttgggtgcgccaggcgcccggcaaggggttggaatatgtcgcagctattagcagtggtagtagcacaggatacggggcagcggtgaagggccgtgccaccatctcgagggacaccgggcagagcacagtgaggctgcagctgaacaacctcagggctgaggacaccgccacctactactgcgccaaaagtggttatggtggtagtgcgagctatgttagcgatatcgacgcatggggccacgggaccgaagtcatcgtctcctcc

SEQ ID NO: 56 - protein, 109 aa, synthetic construct
  REGION 1..109: note = Synthetic: SD-394517_VL
ALALTQPSSVSANPGETVEITCSGGGDYYGTYYYGWYQQKAPGSAPVTVIYDNTKRPSNIPSRFSGSTSGSTNTLTITGVQADDEAVYYCGSYDSSAGIFGAGTTLTVL

SEQ ID NO: 57 - protein, 13 aa, synthetic construct
  REGION 1..13: note = Synthetic: SD-394517_VL_CDR1
SGGGDYYGTYYYG

SEQ ID NO: 58 - protein, 7 aa, synthetic construct
  REGION 1..7: note = Synthetic: SD-394517_VL_CDR2
DNTKRPS

SEQ ID NO: 59 - protein, 9 aa, synthetic construct
  REGION 1..9: note = Synthetic: SD-394517_VL_CDR3
GSYDSSAGI

SEQ ID NO: 60 - DNA, 327 bp, synthetic construct
  misc_feature 1..327: note = Synthetic: SD-394517_VL_DNA
  source 1..327: mol_type = other DNA
gcacttgcgctgactcagccgtcctcggtgtcagcgaacccgggagaaaccgttgagatcacctgctccgggggtggcgactactatggaacttactattatggctggtaccagcagaaggcacctggcagtgcccctgtcactgtgatctatgacaacaccaagagaccctcgaacatcccttcacgattctccggttccacatccggctccacaaacacattaaccatcactggggtccaagccgacgacgaggctgtctattactgtgggagctacgacagcagtgctggtatatttggggccgggacaaccctgaccgtccta

SEQ ID NO: 61 - protein, 123 aa, synthetic construct
  REGION 1..123: note = Synthetic: SD-457798_VH
AVTLDESGGGLQTPGGALSLVCKGSGFTFSSYAMGWVRQAPGKGLEYVAAISSGSSTGYGAAVKGRATISKDTGQSTVRLQLNNLRAEDTATYYCAKSGYGGSASYVSDIDAWGHGTEVIVSS

SEQ ID NO: 62 - protein, 5 aa, synthetic construct
  REGION 1..5: note = Synthetic: SD-457798_VH_CDR1
SYAMG

SEQ ID NO: 63 - protein, 16 aa, synthetic construct
  REGION 1..16: note = Synthetic: SD-457798_VH_CDR2
AISSGSSTGYGAAVKG

SEQ ID NO: 64 - protein, 15 aa, synthetic construct
  REGION 1..15: note = Synthetic: SD-457798_VH_CDR3
SGYGGSASYVSDIDA

SEQ ID NO: 65 - DNA, 369 bp, synthetic construct
  misc_feature 1..369: note = Synthetic: SD-457798_VH_DNA
  source 1..369: mol_type = other DNA
gccgtgacgttggacgagtccgggggcggcctccagacgcccggaggagcgctcagcctcgtctgcaagggctccgggttcaccttcagcagttatgccatgggttgggtgcgccaggcgcccggcaaggggttggaatatgtcgcagctattagcagtggtagtagcacaggatacggggcagcggtgaagggccgtgccaccatctcgaaggacaccgggcagagcacagtgaggctgcagctgaacaacctcagggctgaggacaccgccacctactactgcgccaaaagtggttatggtggtagtgcgagctatgttagcgatatcgacgcatggggccacgggaccgaagtcatcgtctcctcc

SEQ ID NO: 66 - protein, 107 aa, synthetic construct
  REGION 1..107: note = Synthetic: SD-457798_VL
ALALTQPSSVSANPGETVEITCSGGGDYYGTYYYGWYQQKAPGSAPVTVIYDNTKRPSNIPSRFSGSTSGSTNTLTITGVQADDEAIYFCGSYKSNTFGAGTTLTVL

SEQ ID NO: 67 - protein, 13 aa, synthetic construct
  REGION 1..13: note = Synthetic: SD-457798_VL_CDR1
SGGGDYYGTYYYG

SEQ ID NO: 68 - protein, 7 aa, synthetic construct
  REGION 1..7: note = Synthetic: SD-457798_VL_CDR2
DNTKRPS

SEQ ID NO: 69 - protein, 7 aa, synthetic construct
  REGION 1..7: note = Synthetic: SD-457798_VL_CDR3
GSYKSNT

SEQ ID NO: 70 - DNA, 321 bp, synthetic construct
  misc_feature 1..321: note = Synthetic: SD-457798_VL_DNA
  source 1..321: mol_type = other DNA
gcacttgcgctgactcagccgtcctcggtgtcagcgaacccgggagaaaccgttgagatcacctgctccgggggtggcgactactatggaacttactattatggctggtaccagcagaaggcacctggcagtgcccctgtcactgtgatctatgacaacaccaagagaccctcgaacatcccttcacgattctccggttccacatccggctccacaaacacattaaccatcactggggtccaagccgacgacgaggctatctatttctgtgggagctacaaaagtaatacatttggggccgggacaaccctgaccgtccta

SEQ ID NO: 71 - protein, 126 aa, synthetic construct
  REGION 1..126: note = Synthetic: SD-530939_VH
AVTLDESGGGLQTPGGGLSLVCKASGFDFIDYDMFWVRQAPGKGLEWVGVINRSGSYTNYAPAVKGRATISRDNGQSTLRLQLNNLRAEDTGTYYCARGACTHCWDADSAGYIDAWGHGTEVIVSS

SEQ ID NO: 72 - protein, 5 aa, synthetic construct
  REGION 1..5: note = Synthetic: SD-530939_VH_CDR1
DYDMF

SEQ ID NO: 73 - protein, 17 aa, synthetic construct
  REGION 1..17: note = Synthetic: SD-530939_VH_CDR2
VINRSGSYTNYAPAVKG

SEQ ID NO: 74 - protein, 17 aa, synthetic construct
  REGION 1..17: note = Synthetic: SD-530939_VH_CDR3
GACTHCWDADSAGYIDA

SEQ ID NO: 75 - DNA, 378 bp, synthetic construct
  misc_feature 1..378: note = Synthetic: SD-530939_VH_DNA
  source 1..378: mol_type = other DNA
gccgtgacgttggacgagtccgggggcggcctccagacgcctggaggagggctcagcctcgtctgcaaggcctccgggttcgacttcatcgattatgacatgttctgggtgcgacaggctccaggcaaggggctggagtgggtcggtgttattaacaggagtggtagttacacaaactacgcgccggcggtgaagggtcgtgccaccatctcgagggacaacgggcagagcacactgaggctgcagctgaacaacctcagggctgaggacaccggcacctactactgcgccagaggtgcgtgtactcattgttgggatgcagacagtgctggttacatcgacgcatggggccacgggaccgaagtcatcgtctcctcc

SEQ ID NO: 76 - protein, 107 aa, synthetic construct
  REGION 1..107: note = Synthetic: SD-530939_VL
ALALTQPSSVSANPGETVKITCSGGYSGYGWYQQKSPGSAPVTVIYWNDKRPSNIPSRFSGSLSGSTNTLIITGVQAEDEAVYFCGGYDSSPGYVGIFGAGTTLTVL

SEQ ID NO: 77 - protein, 8 aa, synthetic construct
  REGION 1..8: note = Synthetic: SD-530939_VL_CDR1
SGGYSGYG

SEQ ID NO: 78 - protein, 7 aa, synthetic construct
  REGION 1..7: note = Synthetic: SD-530939_VL_CDR2
WNDKRPS

SEQ ID NO: 79 - protein, 12 aa, synthetic construct
  REGION 1..12: note = Synthetic: SD-530939_VL_CDR3
GGYDSSPGYVGI

SEQ ID NO: 80 - DNA, 321 bp, synthetic construct
  misc_feature 1..321: note = Synthetic: SD-530939_VL_DNA
  source 1..321: mol_type = other DNA
gcacttgcgctgactcagccgtcctcagtgtcagcaaacccaggagaaactgtcaagatcacctgctccgggggttacagcggctatggctggtatcagcagaagtcacctggcagtgcccctgtcacagtgatctactggaacgacaagagaccctcaaacatcccttcacgattctccggttccctatccggctccacaaacacattaatcatcactggggtccaagccgaggacgaggctgtctatttctgtggtggctacgacagcagtcctggttatgttggtatatttggggccgggacaaccctgaccgtccta

SEQ ID NO: 81 - protein, 126 aa, synthetic construct
  REGION 1..126: note = Synthetic: SD-589775_VH
AVTLDESGGGLQTPGGGLSLVCKASGFDFIDYDMFWVRQAPGKGLEWVGVINRSGSYTNYAPAVKGRATISRDNGQSTMRLQLNNLRAEDTGTYYCARGACTHCWDADSAGYIDAWGHGTEVIVSS

SEQ ID NO: 82 - protein, 5 aa, synthetic construct
  REGION 1..5: note = Synthetic: SD-589775_VH_CDR1
DYDMF

SEQ ID NO: 83 - protein, 17 aa, synthetic construct
  REGION 1..17: note = Synthetic: SD-589775_VH_CDR2
VINRSGSYTNYAPAVKG

SEQ ID NO: 84 - protein, 17 aa, synthetic construct
  REGION 1..17: note = Synthetic: SD-589775_VH_CDR3
GACTHCWDADSAGYIDA

SEQ ID NO: 85 - DNA, 378 bp, synthetic construct
  misc_feature 1..378: note = Synthetic: SD-589775_VH_DNA
  source 1..378: mol_type = other DNA
gccgtgacgttggacgagtccgggggcggcctccagacgcctggaggagggctcagcctcgtctgcaaggcctccgggttcgacttcatcgattatgacatgttctgggtgcgacaggctccaggcaaggggctggagtgggtcggtgttattaacaggagtggtagttacacaaactacgcgccggcggtgaagggccgtgccaccatctcgagggacaacgggcagagcacgatgaggctgcagctgaacaacctcagggctgaggacaccggcacctactactgcgccagaggtgcgtgtactcattgttgggatgcagacagtgctggttacatcgacgcatggggccacgggaccgaagtcatcgtctcctcc

SEQ ID NO: 86 - protein, 106 aa, synthetic construct
  REGION 1..106: note = Synthetic: SD-589775_VL
ALALTQPSSVSANPGGTVKITCSGSSGSYGWHQQKSPGSAPVTVIYSSDKRPSDIPSRFSGALSGSTATLTITGVRAEDEAVYYCGGYDGSTDVGIFGAGTTLTVL

SEQ ID NO: 87 - protein, 8 aa, synthetic construct
  REGION 1..8: note = Synthetic: SD-589775_VL_CDR1
SGSSGSYG

SEQ ID NO: 88 - protein, 7 aa, synthetic construct
  REGION 1..7: note = Synthetic: SD-589775_VL_CDR2
SSDKRPS

SEQ ID NO: 89 - protein, 11 aa, synthetic construct
  REGION 1..11: note = Synthetic: SD-589775_VL_CDR3
GGYDGSTDVGI

SEQ ID NO: 90 - DNA, 318 bp, synthetic construct
  misc_feature 1..318: note = Synthetic: SD-589775_VL_DNA
  source 1..318: mol_type = other DNA
gcacttgcgctgactcagccgtcctcggtgtcagcaaacccgggaggaaccgtcaagatcacctgctccgggagtagtggcagttatggctggcaccagcagaagtcacctggcagtgcccctgtcactgtgatctatagcagcgacaagagaccctcggacatcccttcacgattctccggtgccctatccggctccacagccacattaaccatcactggggtccgagccgaggacgaggctgtctattactgtggtggctacgacggcagcactgatgttggtatattcggggccgggacaaccctaaccgtccta

SEQ ID NO: 91 - protein, 118 aa, synthetic construct
  REGION 1..118: note = Synthetic: SD-661765_VH
QVQLVETGGDLVKPGGSLKLSCAASGFTFSTYGMSWVRQTPDKRLEWVATISSGGIYTYYSDSVKGRFTISRDNAKNTLYLQMSSLKSEDTAMYYCARDGNYLGFDFWGQGTTLTVSS

SEQ ID NO: 92 - protein, 5 aa, synthetic construct
  REGION 1..5: note = Synthetic: SD-661765_VH_CDR1
TYGMS

SEQ ID NO: 93 - protein, 17 aa, synthetic construct
  REGION 1..17: note = Synthetic: SD-661765_VH_CDR2
TISSGGIYTYYSDSVKG

SEQ ID NO: 94 - protein, 9 aa, synthetic construct
  REGION 1..9: note = Synthetic: SD-661765_VH_CDR3
DGNYLGFDF

SEQ ID NO: 95 - DNA, 354 bp, synthetic construct
  misc_feature 1..354: note = Synthetic: SD-661765_VH_DNA
  source 1..354: mol_type = other DNA
caggtgcagcttgtagagaccgggggagacttagtgaagcctggagggtccctgaaactctcctgtgcagcctctggattcactttcagtacctatggcatgtcttgggttcgccagactccagacaagaggctggagtgggtcgcaaccattagtagtggtggtatttacacctactattcagacagtgtgaaggggcgattcaccatctccagagacaatgccaagaacaccctgtacctgcaaatgagcagtctgaagtctgaggacacagccatgtattactgtgcaagagatggtaactacctgggctttgacttctggggccaaggcaccactctcacagtctcctca

SEQ ID NO: 96 - protein, 111 aa, synthetic construct
  REGION 1..111: note = Synthetic: SD-661765_VL
DIVMTQSPASLAVSLGQRATISCRASESVESYGNSFMNWYQQKPGQPPKLLIYRASNLESGIPARFSGSGSRTDFTLTINPVEADDVATYYCQQSYEDPRTFGGGTKLEIK

SEQ ID NO: 97 - protein, 15 aa, synthetic construct
  REGION 1..15: note = Synthetic: SD-661765_VL_CDR1
RASESVESYGNSFMN

SEQ ID NO: 98 - protein, 7 aa, synthetic construct
  REGION 1..7: note = Synthetic: SD-661765_VL_CDR2
RASNLES

SEQ ID NO: 99 - protein, 9 aa, synthetic construct
  REGION 1..9: note = Synthetic: SD-661765_VL_CDR3
QQSYEDPRT

SEQ ID NO: 100 - DNA, 333 bp, synthetic construct
  misc_feature 1..333: note = Synthetic: SD-661765_VL_DNA
  source 1..333: mol_type = other DNA
gacattgtgatgacacagtctccagcttctttggctgtgtctctagggcagagggccaccatatcctgcagagccagtgaaagtgttgatagttatggcaatagttttatgcactggtaccagcagaaaccaggacagccacccaaactcctcatctatcgtgcatccaacctagaatctgggatccctgccaggttcagtggcagtgggtctaggacagacttcaccctcaccattaatcctgtggaggctgatgatgttgcaacctattactgtcagcaaagtaatgaggatccgtggacgttcggtggaggcaccaagctggaaatcaaa

SEQ ID NO: 101 - protein, 126 aa, synthetic construct
  REGION 1..126: note = Synthetic: SD-685208_VH
AVTLDESGGGLQTPGGALSLVCKASGFDFIDYDMFWVRQAPGKGLEWVGVINRSGSYTNYAPAVKGRATISRDNGQSTMRLQLNNLRAEDTGTYYCARGACTHCWDADSAGYIDAWGHGTEVIVSS

SEQ ID NO: 102 - protein, 5 aa, synthetic construct
  REGION 1..5: note = Synthetic: SD-685208_VH_CDR1
DYDMF

SEQ ID NO: 103 - protein, 17 aa, synthetic construct
  REGION 1..17: note = Synthetic: SD-685208_VH_CDR2
VINRSGSYTNYAPAVKG

SEQ ID NO: 104 - protein, 17 aa, synthetic construct
  REGION 1..17: note = Synthetic: SD-685208_VH_CDR3
GACTHCWDADSAGYIDA

SEQ ID NO: 105 - DNA, 378 bp, synthetic construct
  misc_feature 1..378: note = Synthetic: SD-685208_VH_DNA
  source 1..378: mol_type = other DNA
gccgtgacgttggacgagtccgggggcggcctccagacgcccggaggagcgctcagcctcgtctgcaaggcctccgggttcgacttcatcgattatgacatgttctgggtgcgacaggctccaggcaaggggctggagtgggtcggtgttattaacaggagtggtagttacacaaactacgcgccggcggtgaagggccgtgccaccatctcgagggacaacgggcagagcacaatgaggctgcagctgaacaacctcagggctgaggacaccggcacctactactgcgccagaggtgcgtgtactcattgttgggatgcagacagtgctggttacatcgacgcatggggccacgggaccgaagtcatcgtctcctcc

SEQ ID NO: 106 - protein, 103 aa, synthetic construct
  REGION 1..103: note = Synthetic: SD-685208_VL
ALALTQPSSVSANPGETVKITCSGSSGSYGWFQQKSPGSAPVTVIYWNDKRPSDIPSRFSGSTSGSTGTLTITGVQADDEAVYFCGNADSSGTFGAGTTLTVL

SEQ ID NO: 107 - protein, 8 aa, synthetic construct
  REGION 1..8: note = Synthetic: SD-685208_VL_CDR1
SGSSGSYG

SEQ ID NO: 108 - protein, 7 aa, synthetic construct
  REGION 1..7: note = Synthetic: SD-685208_VL_CDR2
WNDKRPS

SEQ ID NO: 109 - protein, 8 aa, synthetic construct
  REGION 1..8: note = Synthetic: SD-685208_VL_CDR3
GNADSSGT

SEQ ID NO: 110 - DNA, 309 bp, synthetic construct
  misc_feature 1..309: note = Synthetic: SD-685208_VL_DNA
  source 1..309: mol_type = other DNA
gcacttgcgctgactcagccgtcctcggtgtcagcgaacccgggagaaaccgtcaagatcacctgctccgggagtagtggcagctatggctggttccagcagaagtcacctggcagtgcccctgtcactgtgatctattggaatgacaagagaccctcggacatcccttcacgattctccggttccacatccggctccacgggcacattaaccatcactggggtccaagccgacgacgaggctgtctatttctgtgggaatgcagacagcagcggtacatttggggccgggacaaccctgaccgtccta

SEQ ID NO: 111 - protein, 126 aa, synthetic construct
  REGION 1..126: note = Synthetic: SD-802432_VH
AVTLDESGGGLQTPGGGLSLVCKASGFDFIDYDMFWVRQAPGKGLEWVAVINRSGSYTNYAPAVKGRATISRDNGQSTLRLQLNNLRAEDTGTYYCARGACTHCWDADSAGYIDTWGHGTEVIVSS

SEQ ID NO: 112 - protein, 5 aa, synthetic construct
  REGION 1..5: note = Synthetic: SD-802432_VH_CDR1
DYDMF

SEQ ID NO: 113 - protein, 17 aa, synthetic construct
  REGION 1..17: note = Synthetic: SD-802432_VH_CDR2
VINRSGSYTNYAPAVKG

SEQ ID NO: 114 - protein, 17 aa, synthetic construct
  REGION 1..17: note = Synthetic: SD-802432_VH_CDR3
GACTHCWDADSAGYIDT

SEQ ID NO: 115 - DNA, 378 bp, synthetic construct
  misc_feature 1..378: note = Synthetic: SD-802432_VH_DNA
  source 1..378: mol_type = other DNA
gccgtgacgttggacgagtccgggggcggcctccagacgcctggaggagggctcagcctcgtctgcaaggcctccgggttcgacttcatcgattatgacatgttctgggtgcgacaggctccaggcaaggggctggagtgggtcgctgttattaacaggagtggtagttacacaaactacgcgccggcggtgaagggccgtgccaccatctcgagggacaacgggcagagcacactgaggctgcagctgaacaacctcagggctgaggacaccggcacctactactgcgccagaggtgcgtgtactcattgttgggatgcagacagtgctggttacatcgacacatggggccacgggaccgaagtcatcgtctcctcc

SEQ ID NO: 116 - protein, 106 aa, synthetic construct
  REGION 1..106: note = Synthetic: SD-802432_VL
ALALTQPSSVSANPGETVKITCSGGSGSYGWFQQKSPGIAPVIVIFNDDNRPSDIPSRFSGSKSGSTGTLTITGVQAEDEAVYFCGGYDGSTDAGIFGAGTTLTVL

SEQ ID NO: 117 - protein, 8 aa, synthetic construct
  REGION 1..8: note = Synthetic: SD-802432_VL_CDR1
SGGSGSYG

SEQ ID NO: 118 - protein, 7 aa, synthetic construct
  REGION 1..7: note = Synthetic: SD-802432_VL_CDR2
NDDNRPS

SEQ ID NO: 119 - protein, 11 aa, synthetic construct
  REGION 1..11: note = Synthetic: SD-802432_VL_CDR3
GGYDGSTDAGI

SEQ ID NO: 120 - DNA, 318 bp, synthetic construct
  misc_feature 1..318: note = Synthetic: SD-802432_VL_DNA
  source 1..318: mol_type = other DNA
gcacttgcgctgactcagccgtcctcggtgtcagcaaacccaggagaaaccgtcaagatcacctgctccgggggtagtggcagttatggctggttccagcagaagtctcctggcattgcccctgtcattgtgatctttaatgacgacaacagaccgtcggacatcccttcacgattctccggttccaaatccggctccacgggcacattaaccatcactggggtccaagccgaagacgaggctgtctatttctgtggtggctacgacggcagtactgatgctggcatatttggggccgggacaaccctgaccgtccta

SEQ ID NO: 121 - protein, 120 aa, synthetic construct
  REGION 1..120: note = Synthetic: SD-813149_VH
EVKLDESGGGLVKPGGSLKLSCAASGFTFSSYTMSWVRQTPEKRLEWVATINSDGIYTYYPDSVKGRFTISRDNAKNTLYLQMSSLKSEDTAMYYCTRVHYYGYDAMDYWGQGTSVTVSS

SEQ ID NO: 122 - protein, 5 aa, synthetic construct
  REGION 1..5: note = Synthetic: SD-813149_VH_CDR1
SYTMS

SEQ ID NO: 123 - protein, 17 aa, synthetic construct
  REGION 1..17: note = Synthetic: SD-813149_VH_CDR2
TINSDGIYTYYPDSVKG

SEQ ID NO: 124 - protein, 11 aa, synthetic construct
  REGION 1..11: note = Synthetic: SD-813149_VH_CDR3
VHYYGYDAMDY

SEQ ID NO: 125 - DNA, 360 bp, synthetic construct
  misc_feature 1..360: note = Synthetic: SD-813149_VH_DNA
  source 1..360: mol_type = other DNA
gaagtgaagcttgatgagtctgggggaggcttagtgaagcctggagggtccctgaaactctcctgtgcagcctctggattcactttcagtagctataccatgtcttgggttcgccagactccggagaagaggctggagtgggtcgcaactattaatagtgatggtatttacacctactatccagacagtgtgaagggccgattcaccatctccagagacaatgccaagaacaccctgtacctgcaaatgagcagtctgaagtctgaggacacagccatgtattactgtacaagagtacattactacggctacgatgctatggactactggggtcaaggaacctcagtcaccgtctcctca

SEQ ID NO: 126 - protein, 107 aa, synthetic construct
  REGION 1..107: note = Synthetic: SD-813149_VL
EIQMTQSPASLSASVGETVTITCRASENIYSYLAWYQQKQGKSPQLLVYSAKTLAEGVPSRFSGSGSGTHFSLKINSLQPEDFGSYYCQHHYGIPLTFGAGTKLELK

SEQ ID NO: 127 - protein, 11 aa, synthetic construct
  REGION 1..11: note = Synthetic: SD-813149_VL_CDR1
RASENIYSYLA

SEQ ID NO: 128 - protein, 7 aa, synthetic construct
  REGION 1..7: note = Synthetic: SD-813149_VL_CDR2
SAKTLAE

SEQ ID NO: 129 - protein, 9 aa, synthetic construct
  REGION 1..9: note = Synthetic: SD-813149_VL_CDR3
QHHYGIPLT

SEQ ID NO: 130 - DNA, 321 bp, synthetic construct
  misc_feature 1..321: note = Synthetic: SD-813149_VL_DNA
  source 1..321: mol_type = other DNA
gaaatccagatgacccagtctccagcctccctatctgcatctgtgggagaaactgtcaccatcacatgtcgagcaagtgagaatatttacagttatttagcatggtatcagcagaaacagggaaaatctcctcagctcctggtctatagtgcaaaaaccttagcagaaggtgtgccatcaaggttcagtggcagtggatcaggcacacatttttctctgaagatcaacagcctgcagcctgaagattttgggagttattactgtcaacatcattatggtattccgctcacgttcggtgctgggaccaagctggagctgaaa

SEQ ID NO: 131 - protein, 123 aa, synthetic construct; identical to SEQ ID NO: 61
  REGION 1..123: note = Synthetic: SD-970200_VH

SEQ ID NO: 132 - protein, 5 aa, synthetic construct
  REGION 1..5: note = Synthetic: SD-970200_VH_CDR1
SYAMG

SEQ ID NO: 133 - protein, 16 aa, synthetic construct
  REGION 1..16: note = Synthetic: SD-970200_VH_CDR2
AISSGSSTGYGAAVKG

SEQ ID NO: 134 - protein, 15 aa, synthetic construct
  REGION 1..15: note = Synthetic: SD-970200_VH_CDR3
SGYGGSASYVSDIDA

SEQ ID NO: 135 - DNA, 369 bp, synthetic construct; identical to SEQ ID NO: 65
  misc_feature 1..369: note = Synthetic: SD-970200_VH_DNA
  source 1..369: mol_type = other DNA

SEQ ID NO: 136 - protein, 109 aa, synthetic construct; identical to SEQ ID NO: 56
  REGION 1..109: note = Synthetic: SD-970200_VL

SEQ ID NO: 137 - protein, 13 aa, synthetic construct
  REGION 1..13: note = Synthetic: SD-970200_VL_CDR1
SGGGDYYGTYYYG

SEQ ID NO: 138 - protein, 7 aa, synthetic construct
  REGION 1..7: note = Synthetic: SD-970200_VL_CDR2
DNTKRPS

SEQ ID NO: 139 - protein, 9 aa, synthetic construct
  REGION 1..9: note = Synthetic: SD-970200_VL_CDR3
GSYDSSAGI

SEQ ID NO: 140 - DNA, 327 bp, synthetic construct; identical to SEQ ID NO: 60
  misc_feature 1..327: note = Synthetic: SD-970200_VL_DNA
  source 1..327: mol_type = other DNA

SEQ ID NO: 141 - protein, 323 aa, synthetic construct
  REGION 1..323: note = Synthetic: huTROP2
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 142 - protein, 317 aa, synthetic construct
  REGION 1..317: note = Synthetic: msTROP2
MARGLDLAPLLLLLLAMATRFCTAQSNCTCPTNKMTVCDTNGPGGVCQCRAMGSQVLVDCSTLTSKCLLLKARMSARKSGRSLVMPSEHAILDNDGLYDPECDDKGRFKARQCNQTSVCWCVNSVGVRRTDKGDQSLRCDEVVRTHHILIELRHRPTDRAFNHSDLDSELRRLFQERYKLHPSFLSAVHYEEPTIQIELRQNASQKGLRDVDIADAAYYFERDIKGESLFMGRRGLDVQVRGEPLHVERTLIYYLDEKPPQFSMKRLTAGVIAVIAVVSVAVVAGVVVLVVTKRRKSGKYKKVELKELGEMRSEPSL

SEQ ID NO: 143 - protein, 323 aa, synthetic construct
  REGION 1..323: note = Synthetic: cyTROP2
MARGPGLAPPPLRLPLLLLLLAAVTGHTAAQDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRLTAGLIAVIVVVVVALVAGVAVLVISNRRKSGKYKKVEIKELGELRKEPSL

SEQ ID NO: 144 - protein, 314 aa, synthetic construct
  REGION 1..314: note = Synthetic: EPCAM
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 145 - protein, 330 aa, synthetic construct [sequence omitted]

SEQ ID NO: 146 - protein, 107 aa, synthetic construct [sequence omitted]

SEQ ID NO: 147 - protein, 106 aa, synthetic construct
  REGION 1..106: note = Synthetic: huLClambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 148 - protein, 15 aa, synthetic construct
  REGION 1..15: note = Synthetic: Peptide1
MTVCSPDGPGGRCQC

SEQ ID NO: 149 - protein, 15 aa, synthetic construct
  REGION 1..15: note = Synthetic: Peptide2
PDGPGGRCQCRALGS

SEQ ID NO: 150 - protein, 15 aa, synthetic construct
  REGION 1..15: note = Synthetic: Peptide3
DLDAELRRLFRERYR

SEQ ID NO: 151 - protein, 15 aa, synthetic construct
  REGION 1..15: note = Synthetic: Peptide4
LRRLFRERYRLHPKF

SEQ ID NO: 152 - protein, 15 aa, synthetic construct
  REGION 1..15: note = Synthetic: Peptide5
RERYRLHPKFVAAVH

SEQ ID NO: 153 - protein, 15 aa, synthetic construct
  REGION 1..15: note = Synthetic: Peptide6
LHPKFVAAVHYEQPT

SEQ ID NO: 154 - protein, 15 aa, synthetic construct
  REGION 1..15: note = Synthetic: Peptide7
DVDIGDAAYYFERDI

SEQ ID NO: 155 - protein, 15 aa, synthetic construct
  REGION 1..15: note = Synthetic: Peptide8
DAAYYFERDIKGESL

SEQ ID NO: 156 - protein, 126 aa, synthetic construct
  REGION 1..126: note = Synthetic: 589-VH_1
QVQLLESGGGLVKPGGSLRLSCAASGFTFSDYDMFWIRQAPGKGLEWVSVINRSGSYTNYAPAVKGRFTISRDNAKNSLYLQMNSLRAEDTAVYYCARGACTHCWDADSAGYIDAWGQGTLVTVSS

SEQ ID NO: 157 - DNA, 378 bp, synthetic construct
  misc_feature 1..378: note = Synthetic: 589-VH_1 DNA
  source 1..378: mol_type = other DNA
caggtacagctgttggaaagtggaggagggctggtgaagcctggtggcagcctgcggctgagctgcgccgccagcggatttaccttcagtgactatgatatgttctggatccggcaggcccccgggaaaggtttggaatgggtttctgtgattaaccgcagtggcagctatacaaattacgccccggcggtgaaggggaggtttaccataagcagggacaatgcgaagaactctttatacctgcaaatgaacagcctgagggcggaagatacggcagtttactattgcgcacgaggtgcttgtactcattgttgggatgctgactctgctggctatattgacgcgtggggccaggggacactggtgacagttagctct

SEQ ID NO: 158 - protein, 126 aa, synthetic construct
  REGION 1..126: note = Synthetic: 589-VH_2 (SD-861408_VH)
QVQLLESGGGLVKPGGSLRLSCAASGFDFIDYDMFWIRQAPGKGLEWVGVINRSGSYTNYAPAVKGRATISRDNAKNSMYLQMNSLRAEDTAVYYCARGACTHCWDADSAGYIDAWGQGTLVTVSS

SEQ ID NO: 159 - DNA, 378 bp, synthetic construct
  misc_feature 1..378: note = Synthetic: 589-VH_2 DNA
  source 1..378: mol_type = other DNA
caagtacagttgctggagtcagggggtgggttagtgaagccaggaggcagccttcgcttgtcctgcgcagcctctggttttgatttcatcgactacgatatgttctggatccggcaggcccctgggaaagggctggagtgggtaggcgtcatcaacaggtccggttcttataccaattacgcccctgccgtaaagggaagagctacgatcagtcgcgacaacgcgaaaaactctatgtatctccaaatgaattcactgcgggcagaagacacagccgtgtattactgtgcgcggggcgcgtgtacgcattgctgggatgcagactccgccggatacatcgatgcttgggggcagggaactttggtaaccgtgagctcc

SEQ ID NO: 160 - protein, 126 aa, synthetic construct
  REGION 1..126: note = Synthetic: 589-VH_3
QVQLLESGGGLVKPGGSLRLSCAASGFDFIDYDMFWIRQAPGKGLEWVGVINRSGSYTNYAPAVKGRATISRDNGQSTMYLQMNSLRAEDTAVYYCARGACTHCWDADSAGYIDAWGQGTLVTVSS

SEQ ID NO: 161 - DNA, 378 bp, synthetic construct
  misc_feature 1..378: note = Synthetic: 589-VH_3 DNA
  source 1..378: mol_type = other DNA
caggtacaattgctggaaagtggcggagggctggtgaaacctggagggagtctgcggctcagttgtgcagcttctggattcgactttatcgactatgatatgttctggatccgccaggccccgggcaaaggcctcgagtgggttggcgtgatcaatagatcaggtagctatacaaactacgcccctgcggtgaagggcagggccaccattagtcgggacaacggacagtcaacaatgtaccttcagatgaactctttgagggccgaggatacggccgtctactattgcgctcgcggcgcatgtacgcattgctgggacgcggatagtgctggctatatcgatgcgtggggccagggcaccttggtcacagtgagctct

SEQ ID NO: 162 - protein, 126 aa, synthetic construct
  REGION 1..126: note = Synthetic: 589-VH_4
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARGAPTYCSGGSCYGYFDYWGQGTLVTVSS

SEQ ID NO: 163 - DNA, 378 bp, synthetic construct
  misc_feature 1..378: note = Synthetic: 589-VH_4 DNA
  source 1..378: mol_type = other DNA
gaggtacagctcgttgagtcagggggcggcttagtgcagccaggcggctctttaagactctcatgcgccgccagtggcttcacatttagctcctattggatgcactgggtgcgtcaagctcccggcaaaggcctggtctgggtttcccgcatcaacagcgatggcagttccacgtcctatgcagactcagttaagggtcggtttactatctcacgggacaacgcaaagaacaccctgtatctgcagatgaactcactgcgcgcagaggatactgccgtgtactattgtgcaaggggggctcccacctactgtagcggcggcagctgctatggctacttcgactattggggtcaagggaccctggttacagtaagctcc

SEQ ID NO: 164 - protein, 105 aa, synthetic construct
  REGION 1..105: note = Synthetic: 589-VL_1
SYELTQPPSVSVSPGQTASITCSGSSGSYGWYQQKPGQSPVLVIYSSDKRPSGIPERFSGSNSGNTATLTISGTQAMDEADYYCGGYDGSTDVGIFGGGTKLTVL

SEQ ID NO: 165 - DNA, 315 bp, synthetic construct
  misc_feature 1..315: note = Synthetic: 589-VL_1 DNA
  source 1..315: mol_type = other DNA
tcttacgagctgacccagccgccgtcagtctctgtcagtcccggccagaccgctagcattacctgttctggctccagcggatcttatgggtggtatcagcagaagcctggacagtctcctgtccttgtcatatacagctcagacaagcgaccgtcaggcataccggagaggttcagcggctcaaattcaggcaataccgccacactcacaatcagtgggactcaggccatggatgaggcggactactattgcggcggctacgatgggtcaaccgatgtgggtatcttcggcggcggcacaaagctgaccgtgttg

SEQ ID NO: 166 - protein, 105 aa, synthetic construct
  REGION 1..105: note = Synthetic: 589-VL_2 (SD-861408_VL)
SLELTQPPSVSVSPGQTASITCSGSSGSYGWHQQKPGQSPVTVIYSSDKRPSGIPERFSGSSSGSTATLTISGTQAMDEADYYCGGYDGSTDVGIFGGGTKLTVL

SEQ ID NO: 167 - DNA, 315 bp, synthetic construct
  misc_feature 1..315: note = Synthetic: 589-VL_2 DNA
  source 1..315: mol_type = other DNA
tcactcgagcttacgcagccccccagcgttagtgtgagccctggccaaaccgcaagcatcacttgcagcggatcctccgggtcctacggctggcatcagcagaagcccgggcagtctcccgttacagttatatactcatctgataaacgtccatccgggatcccggaaaggttctcaggcagctcaagtggatcaaccgctaccttgactatatctgggacgcaagcaatggatgaggcagattactattgtgggggttacgacgggtctaccgacgtgggtatttttggcggcggcaccaagcttaccgtactt

SEQ ID NO: 168 - protein, 106 aa, synthetic construct
  REGION 1..106: note = Synthetic: 589-VL_3
QSALTQPPSVSASPGQTVTITCSGSSGSYGSWYQQKPGQAPVLVIYEDSKRPSGIPDRFSGSKSGSTATLTISGVQAEDEAVYYCQGYDGSTDVGIFGGGTKLTVL

SEQ ID NO: 169 - DNA, 318 bp, synthetic construct
  misc_feature 1..318: note = Synthetic: 589-VL_3 DNA
  source 1..318: mol_type = other DNA
cagagcgctctgactcagccccccagtgtgtctgcaagccctgggcaaacagtgactatcacatgttccggttcctccgggagttatgggagctggtaccaacagaagccagggcaagccccagtccttgtaatttatgaggacagtaagagaccctctgggatccccgatcggtttagtggcagcaaaagcgggagcacagcaacactgactatcagcggagttcaagcagaggatgaagccgtgtactactgccaggggtatgacggtagtactgacgttgggatattcggaggagggaccaagctcaccgtcctg

SEQ ID NO: 170 - protein, 113 aa, synthetic construct
  REGION 1..113: note = Synthetic: aHEL VH
DVQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSA

SEQ ID NO: 171 - DNA, 339 bp, synthetic construct
  misc_feature 1..339: note = Synthetic: aHEL VH DNA
  source 1..339: mol_type = other DNA
gacgtacaattgcaagaatcaggcccctcacttgtgaaaccgtcacaaactctttcacttacatgttccgtgacaggtgatagtattacctccgattattggtcatggatacgaaaattccctggaaatcggctggagtacatgggttacgtttcttattccggctctacttattacaatcccagtttgaaatctaggattagcatcacgagagatacatctaagaaccagtattatcttgatctgaatagcgttactacggaggacacagccacgtattattgtgcaaattgggacggtgattactggggtcagggtacactggtaacggtctcagcg

SEQ ID NO: 172 - protein, 107 aa, synthetic construct
  REGION 1..107: note = Synthetic: aHEL VL
DIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIK

SEQ ID NO: 173 - DNA, 321 bp, synthetic construct
  misc_feature 1..321: note = Synthetic: aHEL VL DNA
  source 1..321: mol_type = other DNA
gacatcgtacttacccaatctccagcgacattgtcagtaacaccaggaaacagcgtctctctgtcatgtagagcctcacaatctataggaaataatctgcattggtatcagcagaagtcccacgagtcaccgaggttgcttataaagtatgcctcccagtctatttccgggataccatccagattcagtggatctggttccggtacggatttcaccctgagtatcaatagcgtagaaacagaagatttcggtatgtatttttgtcaacagagtaactcctggccctacacattcggaggaggaacgaaactcgagatcaag

SEQ ID NO: 174 - protein, 125 aa, synthetic construct
  REGION 1..125: note = Synthetic: CD3 Gen 1 VH
EVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 175 - DNA, 375 bp, synthetic construct
  misc_feature 1..375: note = Synthetic: CD3 Gen 1 VH DNA
  source 1..375: mol_type = other DNA
gaagtgcagcttgttgaaagcggcggcgggttggtgcagccaggcggcagtctgcggctgtcatgtgctgcttcaggcttcaccttcaatacatatgcaatgaattgggtgcgccaagcgcctggcaagggtttagaatgggtgggaaggatccgcagcaagtataacaattatgcgacatattacgcggattctgtcaaggggagattcacaatctctcgggacgattcaaaaaacactttataccttcaaatgaactcactcagggccgaagatactgctgtctattactgtgtccgacatggtaactttggcaacagttacgtgtcatggttcgcttattggggacaaggaacgctggttactgtctcctca

SEQ ID NO: 176 - protein, 110 aa, synthetic construct
  REGION 1..110: note = Synthetic: CD3 Gen 1 VL
DIQMTQSPSSLSASVGDRVTITCRSSTGAVTTSNYANWVQQKPGKAPKGLIGGTNKRAPGVPARFSGSGSGTDFTLTISSLQPEDFATYYCALWYSNLWVFGQGTKVEIK

SEQ ID NO: 177 - DNA, 330 bp, synthetic construct
  misc_feature 1..330: note = Synthetic: CD3 Gen 1 VL DNA
  source 1..330: mol_type = other DNA
gacatacaaatgactcaatcaccgtcttctttgtccgcgagcgtgggagatcgggtcactataacgtgccgaagctccaccggcgctgtaaccacttccaactatgctaattgggtgcagcaaaagccggggaaggcacctaaaggccttatcgggggaacaaacaagcgcgcacctggagttccagctcggttcagcggttcagggtcaggaaccgactttacgttgacgattagttctcttcagcccgaagattttgccacctattactgtgccctttggtattccaatttgtgggtgtttgggcagggaacaaaggtcgaaattaaa

SEQ ID NO: 178 - protein, 125 aa, synthetic construct
  REGION 1..125: note = Synthetic: SD-385483 VH
EVQLVESGGGLVQPGGSLRLSCAASGFDFNAYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 179 - DNA, 375 bp, synthetic construct
  misc_feature 1..375: note = Synthetic: SD-385483 VH DNA
  source 1..375: mol_type = other DNA
gaggtccaactcgtggaatcaggcgggggattagtgcagcctggcggttcccttaggctctcgtgtgctgcatctggcttcgactttaatgcctacgccatgaactgggtgcgtcaggcacccggaaagggattggagtgggtcggacgcattcgcagcaagtacaataactacgccacttactacgctgattccgtgaaaggccgattcacaatcagccgggacgatagcaaaaacaccctgtatctgcagatgaatagtctgagggccgaagacacagccgtgtactattgcgttcggcacgggaactttggcaattcttatgtatcctggttcgcctactgggggcagggcactctggtcaccgtttctagt

SEQ ID NO: 180 - protein, 110 aa, synthetic construct
  REGION 1..110: note = Synthetic: SD-385483 VL
DIQMTQSPSSLSASVGDRVTITCRSSTGAVTTSNYANWVQQKPGKAPKGLIGGTNKRAPGVPARFSGSGSGTDFTLTISSLQPEDFATYYCAAWHDNAWVFGQGTKVEIK

SEQ ID NO: 181 - DNA, 330 bp, synthetic construct
  misc_feature 1..330: note = Synthetic: SD-385483 VL DNA
  source 1..330: mol_type = other DNA
gatatacaaatgacacagtctccctcttccctctctgcctcagtcggcgatcgcgtgacaattacatgtagatcctccactggtgccgttaccacatccaactacgctaactgggtacagcagaagcctggcaaggcacccaaaggccttattggcgggactaataaacgggcacctggagtcccagccaggttctcgggaagtgggagcgggactgactttaccctgaccatctcaagcttacagccagaggacttcgctacgtattactgcgccgcatggcacgacaacgcttgggtgtttgggcaagggaccaaggtggagatcaaa

SEQ ID NO: 182 - protein, 330 aa, synthetic construct
  REGION 1..330: note = Synthetic: IgG1 hole
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 183 - DNA, 989 bp, synthetic construct
  misc_feature 1..989: note = Synthetic: IgG1 hole DNA
  source 1..989: mol_type = other DNA
gcatccaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacgcgagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcctgctgtacagcaagctgaccgtggacaagagcagatggcagcagggcaacgtgttcagctgcagcgtgatgcacgaggccctgcacaaccactacacccagaagagcctgagcctgagccccggcaa

SEQ ID NO: 184 - protein, 330 aa, synthetic construct
  REGION 1..330: note = Synthetic: IgG1 knob
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 185 - DNA, 990 bp, synthetic construct
  misc_feature 1..990: note = Synthetic: IgG1 knob DNA
  source 1..990: mol_type = other DNA
gcgtcaacaaaaggtccctcagtgtttcctctggccccatcaagcaagagcacctccggcggcaccgccgcactgggatgtctcgtgaaggattacttccctgagcctgtaactgtgagctggaattctggtgcgctgacaagtggtgtccataccttccccgccgtcttgcagagcagtgggctgtatagcttaagcagcgtagtgaccgtccctagctcctccctgggtactcagacttatatctgcaatgtcaatcataaaccctctaataccaaagtcgataaacgcgtggagcctaaatcttgtgataaaacccatacctgcccaccatgtcccgcccctgagctgctcggcggtccatccgtcttcctgtttcctccaaaaccaaaggacacattgatgattagtaggacacccgaggtcacctgtgtggtggttgacgtttcccacgaggacccagaggtcaaattcaactggtatgtggacggcgtggaggtccacaacgctaaaaccaaacccagagaagaacagtacgcctctacttatagagttgtgagcgtcctcacggtgctgcaccaggattggctgaatggcaaggagtataagtgcaaagtctctaataaagcactccctgcgccaatagagaagacaattagcaaggcaaagggacaaccacgcgaacctcaggtgtacactctcccccctagccgggaagagatgacaaaaaatcaggtttctttaacatgtctggtgaaaggattttacccttccgacatcgcggtcgaatgggagtcaaatggacaacccgagaataattacaagacaacaccaccagtgttagactccgatggctccttctttctgtacagcaggctgacggtggacaagagccgctggcaacaggggaatgtcttctcttgctctgtgatgcatgaggcactccacaatcattatacgcagaaaagtctctctctctctccaggcaaa

SEQ ID NO: 186 - protein, 455 aa, synthetic construct
  REGION 1..455: note = Synthetic: SD-174078_HC_knob AA
EVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 187 - DNA, 1365 bp, synthetic construct
  misc_feature 1..1365: note = Synthetic: SD-174078_HC_knob DNA
  source 1..1365: mol_type = other DNA
gaagtgcagcttgttgaaagcggcggcgggttggtgcagccaggcggcagtctgcggctgtcatgtgctgcttcaggcttcaccttcaatacatatgcaatgaattgggtgcgccaagcgcctggcaagggtttagaatgggtgggaaggatccgcagcaagtataacaattatgcgacatattacgcggattctgtcaaggggagattcacaatctctcgggacgattcaaaaaacactttataccttcaaatgaactcactcagggccgaagatactgctgtctattactgtgtccgacatggtaactttggcaacagttacgtgtcatggttcgcttattggggacaaggaacgctggttactgtctcctcagcgtcaacaaaaggtccctcagtgtttcctctggccccatcaagcaagagcacctccggcggcaccgccgcactgggatgtctcgtgaaggattacttccctgagcctgtaactgtgagctggaattctggtgcgctgacaagtggtgtccataccttccccgccgtcttgcagagcagtgggctgtatagcttaagcagcgtagtgaccgtccctagctcctccctgggtactcagacttatatctgcaatgtcaatcataaaccctctaataccaaagtcgataaacgcgtggagcctaaatcttgtgataaaacccatacctgcccaccatgtcccgcccctgagctgctcggcggtccatccgtcttcctgtttcctccaaaaccaaaggacacattgatgattagtaggacacccgaggtcacctgtgtggtggttgacgtttcccacgaggacccagaggtcaaattcaactggtatgtggacggcgtggaggtccacaacgctaaaaccaaacccagagaagaacagtacgcctctacttatagagttgtgagcgtcctcacggtgctgcaccaggattggctgaatggcaaggagtataagtgcaaagtctctaataaagcactccctgcgccaatagagaagacaattagcaaggcaaagggacaaccacgcgaacctcaggtgtacactctcccccctagccgggaagagatgacaaaaaatcaggtttctttaacatgtctggtgaaaggattttacccttccgacatcgcggtcgaatgggagtcaaatggacaacccgagaataattacaagacaacaccaccagtgttagactccgatggctccttctttctgtacagcaggctgacggtggacaagagccgctggcaacaggggaatgtcttctcttgctctgtgatgcatgaggcactccacaatcattatacgcagaaaagtctctctctctctccaggcaaa

SEQ ID NO: 188 - protein, 450 aa, synthetic construct
  REGION 1..450: note = Synthetic: SD-174078_HC_hole AA
EVKLDESGGGLVKPGGSLKLSCAASGFTFSSYTMSWVRQTPEKRLEWVATINSDGIYTYYPDSVKGRFTISRDNAKNTLYLQMSSLKSEDTAMYYCTRVHYYGYDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 189 - DNA, 1350 bp, synthetic construct
  misc_feature 1..1350: note = Synthetic: SD-174078_HC_hole DNA
  source 1..1350: mol_type = other DNA
gaagtgaagcttgatgagtctgggggaggcttagtgaagcctggagggtccctgaaactctcctgtgcagcctctggattcactttcagtagctataccatgtcttgggttcgccagactccggagaagaggctggagtgggtcgcaactattaatagtgatggtatttacacctactatccagacagtgtgaagggccgattcaccatctccagagacaatgccaagaacaccctgtacctgcaaatgagcagtctgaagtctgaggacacagccatgtattactgtacaagagtacattactacggctacgatgctatggactactggggtcaaggaacctcagtcaccgtctcctcagccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacgcgagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcctgctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 190 - protein, 455 aa, synthetic construct
  REGION 1..455: note = Synthetic: SD-231831_HC_knob AA
EVQLVESGGGLVQPGGSLRLSCAASGFDFNAYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 191 - DNA, 1365 bp, synthetic construct
  misc_feature 1..1365: note = Synthetic: SD-231831_HC_knob DNA
  source 1..1365: mol_type = other DNA
gaggtccaactcgtggaatcaggcgggggattagtgcagcctggcggttcccttaggctctcgtgtgctgcatctggcttcgactttaatgcctacgccatgaactgggtgcgtcaggcacccggaaagggattggagtgggtcggacgcattcgcagcaagtacaataactacgccacttactacgctgattccgtgaaaggccgattcacaatcagccgggacgatagcaaaaacaccctgtatctgcagatgaatagtctgagggccgaagacacagccgtgtactattgcgttcggcacgggaactttggcaattcttatgtatcctggttcgcctactgggggcagggcactctggtcaccgtttctagtgcgtcaacaaaaggtccctcagtgtttcctctggccccatcaagcaagagcacctccggcggcaccgccgcactgggatgtctcgtgaaggattacttccctgagcctgtaactgtgagctggaattctggtgcgctgacaagtggtgtccataccttccccgccgtcttgcagagcagtgggctgtatagcttaagcagcgtagtgaccgtccctagctcctccctgggtactcagacttatatctgcaatgtcaatcataaaccctctaataccaaagtcgataaacgcgtggagcctaaatcttgtgataaaacccatacctgcccaccatgtcccgcccctgagctgctcggcggtccatccgtcttcctgtttcctccaaaaccaaaggacacattgatgattagtaggacacccgaggtcacctgtgtggtggttgacgtttcccacgaggacccagaggtcaaattcaactggtatgtggacggcgtggaggtccacaacgctaaaaccaaacccagagaagaacagtacgcctctacttatagagttgtgagcgtcctcacggtgctgcaccaggattggctgaatggcaaggagtataagtgcaaagtctctaataaagcactccctgcgccaatagagaagacaattagcaaggcaaagggacaaccacgcgaacctcaggtgtacactctcccccctagccgggaagagatgacaaaaaatcaggtttctttaacatgtctggtgaaaggattttacccttccgacatcgcggtcgaatgggagtcaaatggacaacccgagaataattacaagacaacaccaccagtgttagactccgatggctccttctttctgtacagcaggctgacggtggacaagagccgctggcaacaggggaatgtcttctcttgctctgtgatgcatgaggcactccacaatcattatacgcagaaaagtctctctctctctccaggcaaa

SEQ ID NO: 192 - protein, 456 aa, synthetic construct
  REGION 1..456: note = Synthetic: SD-231831_HC_hole AA
QVQLLESGGGLVKPGGSLRLSCAASGFDFIDYDMFWIRQAPGKGLEWVGVINRSGSYTNYAPAVKGRATISRDNAKNSMYLQMNSLRAEDTAVYYCARGACTHCWDADSAGYIDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 193 - DNA, 1368 bp, synthetic construct
  misc_feature 1..1368: note = Synthetic: SD-231831_HC_hole DNA
  source 1..1368: mol_type = other DNA
caagtacagttgctggagtcagggggtgggttagtgaagccaggaggcagccttcgcttgtcctgcgcagcctctggttttgatttcatcgactacgatatgttctggatccggcaggcccctgggaaagggctggagtgggtaggcgtcatcaacaggtccggttcttataccaattacgcccctgccgtaaagggaagagctacgatcagtcgcgacaacgcgaaaaactctatgtatctccaaatgaattcactgcgggcagaagacacagccgtgtattactgtgcgcggggcgcgtgtacgcattgctgggatgcagactccgccggatacatcgatgcttgggggcagggaactttggtaaccgtgagctccgcatccaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacgcgagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcctgctgtacagcaagctgaccgtggacaagagcagatggcagcagggcaacgtgttcagctgcagcgtgatgcacgaggccctgcacaaccactacacccagaagagcctgagcctgagccccggcaag